(19) United States
(12) Patent Application Publication
(10) Pub. No.: US 2025/0213191 A1
(43) Pub. Date: Jul. 3, 2025

(54) COMPRESSION EVENT DETECTION AND PREVENTION

(71) Applicant: Dexcom, Inc., San Diego, CA (US)

(72) Inventors: Zachary Felix WOOD, San Diego, CA (US); Caleb Michael CHRISTIANSON, San Diego, CA (US); Neal JOHNSTON, San Diego, CA (US); Mark D. KEMPKEY, San Diego, CA (US); Rui MA, San Diego, CA (US); Nicholas Robert MEDJO, San Diego, CA (US); Milan RAJ, San Diego, CA (US); William Eric REYNA, San Diego, CA (US); Harleen Sarah Kaur SINGH, San Diego, CA (US); Jeffrey James SMITH, San Diego, CA (US)

(21) Appl. No.: 18/962,703

(22) Filed: Nov. 27, 2024

Related U.S. Application Data
(60) Provisional application No. 63/616,456, filed on Dec. 29, 2023.

(51) Int. Cl. A61B 5/00; A61B 5/145; A61B 90/00

(52) U.S. Cl. CPC A61B 5/6843; A61B 5/14532; A61B 5/14546; A61B 2090/064

(57) ABSTRACT

A continuous analyte monitoring system includes analyte sensors configured to sense analytes such as lactate and glucose in the tissue of a user. A controller is coupled to the analyte sensors and configured evaluate first samples of outputs of a first analyte sensor and second samples of outputs of a second analyte sensor to determine whether the first samples and the second samples indicate compression of the tissue. The controller compensates for the compression of the tissue with respect to the first samples. A force sensor may be used and may be positioned between a circuit board and a housing, the circuit board supported by supports providing preloading of the force sensor. A force deflector may be used to direct loads away from tissue holding the analyte sensors. A housing may have a flexible lower surface to reduce loading of the tissue.

### CROSS-REFERENCE TO RELATED APPLICATIONS

This application claims the benefit of U.S. Provisional Application No. 63/616,456, filed Dec. 29, 2023, the entire contents of which are incorporated by reference herein.

### INTRODUCTION

Diabetes mellitus is a metabolic condition relating to the production or use of insulin by the body. Insulin is a hormone that allows the body to use glucose for energy, or store glucose as fat.

When a person eats a meal that contains carbohydrates, the digestive system absorbs nutrients, ultimately depositing glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), skin ulcers, and kidney damage. Hyperglycemia can also lead to acute problems, such as diabetic ketoacidosis-a state in which the body becomes excessively acidic due to the production of excess ketones, or body acids. The state of having lower than normal blood glucose levels is called “hypoglycemia.” Severe hypoglycemia can lead to damage of the heart muscle, neurocognitive dysfunction, and in certain cases, acute crises that can result in seizures or even death.

A patient living with diabetes can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Patients with diabetes can benefit from real-time diabetes management guidance, as determined based on a physiological state of the patient, in order to stay within a target glucose range and avoid physical complications. In certain cases, the physiological state of the patient is determined using monitoring systems that measure glucose levels, which inform the identification and/or prediction of adverse glycemic events, such as hyperglycemia and hypoglycemia, and the type of guidance provided to the patient.

For example, such monitoring systems may utilize a continuous glucose monitor (CGM) to measure a patient's glucose levels over time. The measured glucose levels may then be processed by the monitoring system to identify and/or predict adverse glycemic events, and/or to provide guidance to the patient for treatment and or actions to abate or prevent the occurrence of such adverse glycemic events. For example, trends, statistics, or other metrics may be derived from the glucose levels and used to identify and/or predict adverse glycemic events. Or, in certain cases, the glucose levels themselves may be used to identify and/or predict adverse glycemic events.

A CGM includes a sensor mounted to the body of the patient, such as to the arm or torso. The sensor includes electrodes coated in enzymes that are in contact with the interstitial fluid of the patient, each enzyme reacting with an analyte to be sensed, such as glucose, lactate, or others. When an analyte reacts with the enzyme on an electrode, a detectable current is induced with the magnitude of the current corresponding to the concentration of the analyte.

However, the detected current from a given electrode may be influenced by factors other than the concentration of the analyte corresponding to the enzyme coating the electrode. For example, the sensor may also unintentionally detach from the patient, the electrodes or circuits connected to them may be damaged, etc. Absent a failure or detachment of the sensor itself, the detected current may also be affected by compression of the sensor itself due to the patient sitting or lying on the sensor or some other source of pressure. Compression of the sensor may cause the output of the sensor to inaccurately indicate analyte levels or sensor failure.

Accordingly, there is a need in the art for improved systems and methods for accurately detecting failure of a CGM sensor as well as detecting and preventing compression events.

### SUMMARY

In certain embodiments, an on-skin wearable medical device is provided that includes a housing configured to be worn over skin of a user and one or more electronic components disposed within the housing. The on-skin wearable medical device further includes one or more analyte sensors electrically connected to the one or more electronic components and configured to sense one or more analytes within tissue of a user. A force sensor is operably coupled to the housing and electrically connected to the one or more electronic components.

In certain embodiments, a method of detecting compressive forces on an on-skin wearable medical device is provided. The method includes providing a housing configured to be work over skin of a user, the housing comprising a controller coupled to one or more analyte sensors configured to sense one or more analytes within tissue of the user; providing a force sensor operably coupled to the housing and to the controller, the force sensor configured to sense loading of the housing; receiving, by the controller, one or more outputs of the one or more analyte sensors; receiving, by the controller, an output of the force sensor; and adjusting, by the controller, the one or more outputs of the one or more analyte sensors according to the output of the force sensor.

In certain embodiments, a force deflector for deflecting compressive forces away from an on-skin wearable medical device is provided. The force deflector includes a first portion configured to rest on skin of a user, and a second portion configured to rest on the skin of the user, the second portion separated from the first portion by a first gap. The force deflector further comprises a central portion secured to the first portion and the second portion and configured to define a second gap between the skin of the user and a lower surface of the central portion.

In certain embodiments, method of deflecting compressive forces away from an on-skin wearable medical device is provided. The method includes providing the on-skin wearable medical device and providing a force deflector configured to be placed on skin of a user and over the on-skin wearable medical device. The force deflector comprises a first portion configured to rest on the skin of the user and a second portion configured to rest on the skin of the user, the second portion separated from the first portion by a first gap. The force deflector further comprises a central portion secured to the first portion and the second portion and offset from the on-skin wearable medical device by a second gap.

In certain embodiments, an on-skin wearable medical device is provided. The on-skin wearable medical device includes a housing configured to be worn over skin of a user, the housing comprising a lower surface configured to face the skin of the user and comprising a flexible material. The on-skin wearable medical device further comprises one or more electronic components disposed within the housing, and one or more analyte sensors coupled to the one or more electronic components and configured to be inserted into tissue of the user.

In certain embodiments, an on-skin wearable medical device is provided. The on-skin wearable medical device comprises a sensor having a distal end portion and a proximal end portion, the distal end portion configured to be inserted into skin of a host. The on-skin wearable medical device comprises one or more electronic components operably coupled to the sensor. The on-skin wearable medical device comprises a lower membrane defining an opening. The on-skin wearable medical device comprises an upper membrane coupled to the lower membrane, the upper membrane comprising a central portion and a sidewall extending around the central portion, the sidewall extending to the lower membrane, the upper membrane defining an inner wall extending between the central portion and the opening of the lower membrane, wherein the one or more electronic components and the proximal end portion of the sensor are disposed between the upper membrane and the lower membrane, and wherein the inner wall and the opening of the lower membrane together define a channel for receiving an insertion member configured to insert the distal end portion into the skin of the host.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates aspects of an example disease management system used in connection with implementing embodiments of the present disclosure.

FIG. 2 is a diagram conceptually illustrating an example continuous analyte monitoring system including example continuous analyte sensors with sensor electronics, in accordance with certain aspects of the present disclosure.

FIG. 3 is a block diagram illustrating electrodes of a continuous analyte monitoring system, according to certain embodiments disclosed herein.

FIG. 4 is a block diagram that illustrates electronics associated with the continuous analyte monitoring system of FIG. 2, according to certain embodiments disclosed herein.

FIG. 5 illustrates plots of the current output by glucose and lactate analyte sensors during a compression event, according to certain embodiments disclosed herein.

FIG. 6 illustrates plots of the current output by glucose and lactate analyte sensors in response to an acute compression event, according to certain embodiments disclosed herein.

FIG. 7 is a diagram illustrating the flow of data with respect to a sensor failure detection module, according to certain embodiments disclosed herein.

FIG. 8 illustrates the use of a machine learning model to identify compression events, according to certain embodiments disclosed herein.

FIG. 9 is a process flow diagram of a method for determining whether to perform processing to compensate for compression events, according to certain embodiments disclosed herein.

FIG. 10 is a process flow diagram of a method for compensating for compression events, according to certain embodiments disclosed herein.

FIGS. 11A-11C illustrate an on-skin wearable medical device having a transcutaneous analyte sensor.

FIG. 12A illustrates a perspective view of an on-skin wearable medical device.

FIG. 12B illustrates a top view of the on-skin wearable medical device shown in FIG. 12A.

FIG. 12C illustrates an exploded view of the on-skin wearable medical device of FIG. 12A.

FIG. 12D illustrates a cross-sectional view of a section of the on-skin wearable medical device of FIG. 12A.

FIG. 12E illustrates a cross-sectional exploded view of a section of the on-skin wearable medical device of FIG. 12A.

FIG. 12F illustrates a cross-sectional view of a section of the on-skin wearable medical device shown in FIG. 12E.

FIG. 13A illustrates a top perspective view of a socket and a plug of an on-skin wearable medical device with the plug detached from the socket.

FIG. 13B illustrates a bottom perspective view of the socket and the plug of the on-skin wearable medical device shown in FIG. 13A with the plug detached from the socket.

FIG. 13C illustrates an isolated bottom perspective view of the plug of FIG. 13A.

FIG. 13D illustrates a magnified cross-sectional view of the plug of FIG. 13A attached to the socket of FIG. 13A.

FIG. 14A illustrates a top perspective view of an on-skin wearable medical device.

FIG. 14B illustrates a side perspective view of the on-skin wearable medical device of FIG. 14A.

FIG. 14C illustrates a top perspective view of the on-skin wearable medical device of FIG. 14A with a detached plug.

FIG. 14D illustrates a bottom perspective view of the plug of FIG. 14C.

FIG. 14E illustrates a top perspective exploded view of the on-skin wearable medical device of FIG. 14A.

FIG. 14F illustrates a bottom perspective exploded view of the on-skin wearable medical device of FIG. 14A.

FIG. 14G illustrates an isolated perspective view of a first portion of a housing of the on-skin wearable medical device.

FIG. 14H illustrates an isolated bottom perspective view of the first portion of the housing of the on-skin wearable medical device of FIG. 14G.

FIG. 14I illustrates an isolated perspective view of the housing of the on-skin wearable medical device of FIG. 14A.

FIG. 15A illustrates a perspective view of an on-skin wearable medical device.

FIG. 15B illustrates an exploded view of an on-skin wearable medical device.

FIG. 16 illustrates an exploded view of an on-skin wearable medical device.

FIG. 17 illustrates an exploded view of an on-skin wearable medical device.

FIGS. 18A to 18C illustrate a continuous analyte sensor system including a force sensor, according to certain embodiments disclosed herein.

FIGS. 19A and 19B illustrate layout of components within a continuous analyte sensor system, according to certain embodiments disclosed herein.

FIG. 20 illustrates an example column cross section for supporting a circuit board of a continuous analyte sensor system, according to certain embodiments disclosed herein.

FIGS. 21A and 21B illustrate a first embodiment of leaf springs for supporting the circuit board of the continuous analyte sensor system, according to certain embodiments disclosed herein.

FIGS. 22A and 22B illustrate a second embodiment of leaf springs for supporting the circuit board of the continuous analyte sensor system, according to certain embodiments disclosed herein.

FIGS. 23A and 23B illustrate a third embodiment of leaf springs for supporting the circuit board of the continuous analyte sensor system, according to certain embodiments disclosed herein.

FIGS. 24A and 24B illustrate continuous analyte sensor systems comprising a compressible member, according to certain embodiments disclosed herein.

FIGS. 25A and 25B illustrate electrode configurations for a force sensitive resistor including a conductive member, according to certain embodiments disclosed herein.

FIGS. 26A to 26C illustrate cross-sectional shapes of conductive foam for use in a force sensitive resistor, according to certain embodiments disclosed herein.

FIGS. 27A and 27B illustrate cross-sectional shapes of conductive foam along section planes perpendicular to an applied force for use in a force sensitive resistor, according to certain embodiments disclosed herein.

FIGS. 28A to 28D are plots of the output of a force sensitive resistor with respect to force, according to certain embodiments disclosed herein.

FIGS. 29A to 29F illustrate alternative approaches for sensing force applied to a continuous analyte sensor system, according to certain embodiments disclosed herein.

FIGS. 30A and 30B illustrate a force deflector for use with a continuous analyte sensor system, according to certain embodiments disclosed herein.

FIGS. 31A to 31F illustrate an alternative embodiment of a force deflector for use with a continuous analyte sensor system, according to certain embodiments disclosed herein.

FIGS. 32A and 32B are plots of the output of a continuous analyte sensor system with and without use of a force deflector, according to certain embodiments disclosed herein.

FIGS. 33A and 33B illustrate a housing for a continuous analyte sensor system having a compliant lower surface, according to certain embodiments disclosed herein.

FIGS. 34A and 34B illustrate another housing for a continuous analyte sensor system having a compliant lower surface, according to certain embodiments disclosed herein.

FIGS. 35A and 35B illustrate yet another housing for a continuous analyte sensor system having a compliant lower surface, according to certain embodiments disclosed herein.

FIGS. 36A to 36C illustrate a continuous analyte sensor system with a sealed housing incorporating structural foam and a compliant lower surface, according to certain embodiments disclosed herein.

FIGS. 37A and 37B illustrate a continuous analyte sensor system with a sealed housing and an outer housing providing mechanical support, according to certain embodiments disclosed herein.

FIG. 38 illustrates a perspective view of a unit including a conductive member and a coupling member.

FIG. 39 illustrates a side view of the unit shown in FIG. 38.

FIG. 40 illustrates a perspective assembly view of a wearable device including the unit of FIG. 38.

FIG. 41 illustrates a perspective view of the unit of FIG. 38 positioned upon a circuit board.

FIG. 42 illustrates a side cross sectional view of the wearable device of FIG. 40.

FIG. 43 illustrates a perspective schematic view of an elongate sheet of materials.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

As described above, compression of an analyte sensor may cause the output of an analyte sensor to inaccurately measure an analyte, such as glucose. Accordingly, systems and methods according to the present principles relate to detecting and compensating for compression events for a continuous analyte monitoring system. For example, embodiments provided herein include improved systems and methods for detecting compression events using multiple analyte sensors, such as glucose and lactate. Various embodiments of force sensors and structures for reducing the effect of compression are also disclosed. Such systems and methods help avoid disturbing the patient with false alarms while still promoting patient safety. Accordingly, the present disclosure provides a technical solution to the technical problems described above by providing techniques for accurately detecting compression events.

Specifically, a continuous analyte monitoring system is described that evaluates both lactate and glucose samples. For example, in response to detecting a force exceeding a threshold, expected values for lactate and glucose samples may be compared to the actual lactate and glucose samples. The expected values may be calculated using a filter, which may use a function of past values for the glucose and lactate samples and possibly other inputs such as force, exercise, diet, and drug data. If differences between the actual lactate and glucose samples have an inverse correlation with respect to the expected values for the lactate and glucose sample, a compression event may be deemed to have occurred and the glucose samples may be adjusted. If an inverse correlation is not found, an acute compression event may be deemed to have occurred and the glucose samples may be blanked.

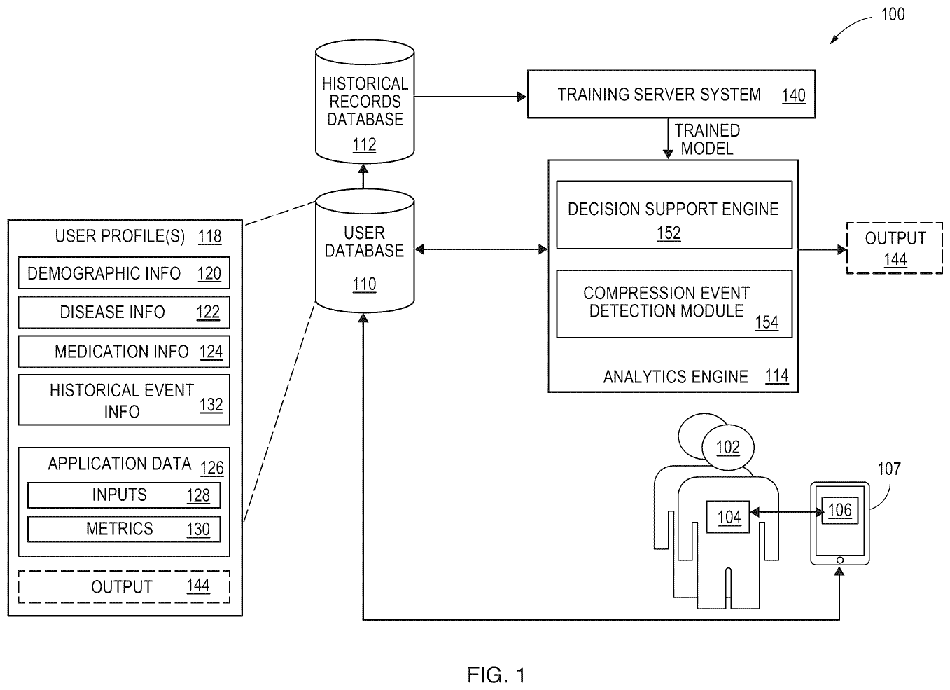

FIG. 1 illustrates an example disease management system 100 for assisting users 102 (individually referred to herein as a user and collectively referred to herein as users) with decision support for managing a disease, e.g., diabetes, kidney disease, liver disease, or other types of diseases. The system 100 utilizes a continuous analyte monitoring system 104 that continuously measures one or more of a plurality of analytes, such as glucose, lactate, potassium, creatinine, ketone, alcohol, etc. A user may be a patient or, in some cases, the patient's caregiver. In certain embodiments, the system 100 includes the continuous analyte monitoring system 104, a display device 107 that executes an application 106, analytics engines 114, a user database 110, a historical records database 112, and a training server system 140, each of which is described in more detail below.

The analytes that may be measured and analyzed by the devices and methods described herein include glucose, lactate, ketones, potassium, and in some examples, other analytes listed above. However, other analytes, which are not listed above, may also be considered.

An analyte may be a substance or chemical constituent in a biological fluid (for example, blood, interstitial fluid, cerebral spinal fluid, lymph fluid, sweat, or urine) that can be analyzed. Analytes can include naturally occurring substances, artificial substances, metabolites, or reaction products. Analytes for measurement by the devices and methods may include, but may not be limited to, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; e reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycerol; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; potassium, quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

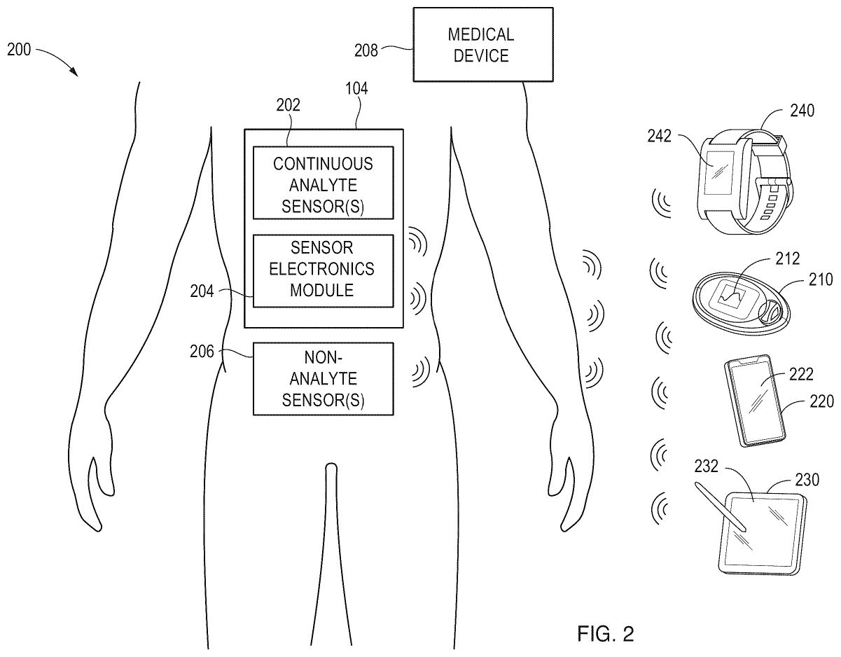

The continuous analyte monitoring system 104 may continuously measure one or more analytes and transmit the analyte measurements to the display device 107 for use by the application 106. In some embodiments, the continuous analyte monitoring system 104 transmits the analyte measurements to the display device 107 through a wireless connection (e.g., Bluetooth connection). The display device 107 may be a smart phone, a laptop computer, a smart watch, a fitness tracker, a tablet, or any other computing device capable of executing the application 106. The continuous analyte monitoring system 104 may be described in more detail with respect to FIG. 2.

The application 106 may be a mobile health application that receives and analyzes analyte measurements from the analyte monitoring system 104. For example, the application 106 stores information about a user, including the user's analyte measurements, in a user profile 118 of the user for processing and analysis, as well as for use by the decision support engine 152 to provide decision support recommendations or guidance to the user.

The analytics engines 114 include a decision support engine 152 providing disease management decision support recommendations to the user, e.g., via the application 106. The decision support engine 152 may provide such recommendations based on analyte measurements for one or more analytes received from the continuous analyte monitoring system 104, data obtained from one or more non-analyte sensors or devices, or information included in the user profile 118. In certain embodiments, the analytics engines 114 execute entirely on one or more computing devices in a private or a public cloud. In such embodiments, the application 106 communicates with the analytics engines 114 over a network (e.g., Internet). In some other embodiments, the analytics engines 114 execute partially on one or more local devices, such as the display device 107 or a processor of the continuous analyte monitoring system 104, and partially on one or more computing devices in a private or a public cloud. In some other embodiments, the analytics engines 114 execute entirely on one or more local devices, such as the display device 107 or a processor of the continuous analyte monitoring system 104.

The analytics engines 114 may include a compression event detection module 154. The compression event detection module 154 analyzes signals from the continuous analyte monitoring system 104 and determines whether the signals indicate a compression event. The compression event detection module 154 further implements methods described herein for distinguishing between compensatable compression events and acute compression events. During a compression event, compression of the tissue around the continuous analyte monitoring system 104 may cause the signals output therefrom to temporarily be very low or otherwise outside the range of values expected during normal operation. The compression event detection module 154 is therefore configured to identify such compression events and, in response, adjust and/or blank data recorded during compression events that is passed on to the decision support engine 152 or other consumer of the output of the continuous analyte monitoring system 104.

The user profile 118 may include information collected about the user from the application 106. For example, the application 106 provides a set of inputs 128, including the analyte measurements for the one or more analytes received from the continuous analyte monitoring system 104 that are stored in the user profile 118. In certain embodiments, inputs 128 provided by the application 106 include other data in addition to the analyte measurements. For example, the application 106 may obtain additional inputs 128 through manual user input, one or more other non-analyte sensors or devices (e.g., temperature sensors, etc.), other applications executing on the display device 107, etc. Non-analyte sensors and devices include one or more of, but are not limited to, an insulin pump, respiratory sensor, sensors or devices provided by the display device 107 (e.g., accelerometer, gyrometer, camera, global positioning system (GPS), heart rate monitor, etc.) or other user accessories (e.g., a smart watch), or any other sensors or devices that provide relevant information about the user. The inputs 128 of the user profile 118 provided by the application 106 may, for example, include continuous analyte sensor data, non-analyte sensor data, time, food consumption, physical activity, sleep information, user statistics, medication, etc.

The user profile 118 further includes demographic information 120, disease information 122, or medication information 124. Such information may be provided through user input or obtained from certain data stores (e.g., electronic medical records, etc.). The demographic information 120 may include one or more of the user's age, BMI (body mass index), ethnicity, gender, etc. The disease information 122 may include information about one or more diseases of a user, including relevant information pertaining to the user's condition of diabetes or other conditions (e.g., liver disease, kidney disease, etc.). The disease information 122 may also include the length of time since diagnosis, the level of disease control, level of compliance with disease management therapy, other types of diagnoses (e.g., heart disease, obesity), etc. The disease information 122 may include other measures of health (e.g., heart rate, stress, sleep, etc.) or fitness (e.g., cardiovascular endurance, muscular strength or power, muscular endurance, and other measures of fitness), or the like.

The medication information 124 may include information about the amount and type of a medication taken by a user. For example, the medication information 124 may include information about the consumption of one or more drugs for management of the user's condition of diabetes, such as insulin (e.g., short-acting insulin, rapid-acting insulin (insulin aspart, insulin gluilisine, insulin lispro), intermediate-acting insulin (insulin isophane), long-acting insulin degludec, indulin detemir, insulin glargine, insulin), combination insulins), amylinomimetic drugs, alpha-glucosidase inhibitors (e.g., acarbose, miglitol), biguanides (e.g., metformin-alogliptin, metformin-canagliflozin, metformin-dapagliflozin, metformin-empagliflozin, metformin-glipizide, metformin-glyburide, metformin-linagliptin, metformin-pioglitazone, metformin-repaglinide, metformin-rosiglitazone, metformin-saxagliptin, metformin-sitagliptin), dopamine agonists (e.g., bromocriptine), dipeptidyl peptidase-4 (DPP-4) inhibitors (e.g., alogliptin, alogliptin-pioglitazone, linagliptin, linagliptin-empagliflozin, saxagliptin, sitagliptin, simvastatin), glucagon-like peptide-1 receptor agonists (GLP-1 receptor agonists) (e.g., albiglutide, dulaglutide, exenatide, liraglutide, semaglutide), meglitinides (e.g., nateglinide, repaglinide), sodium-glucose transporter (SGLT) 2 inhibitors (e.g., dapagliflozin, canagliflozin, empagliflozin, ertugliflozin), sulfonylureas (e.g., glimepiride, glimepiride-pioglitazone, glimepiride-rosiglitazone, gliclazide, glipizide, glyburide, chlorpropamide, tolazamide, tolbutamide), thiazolidinediones (e.g., rosiglitazone, pioglitazone), and other drugs. The medication information 124 may include information about the consumption of one or more drugs for management or treatment of other diseases or conditions of the user, including drugs for cholesterol, high blood pressure, heart disease, etc., in addition to supplements to promote general health and metabolism, such as vitamins.

Data stored in the user profile 118 may maintain time series data collected for the user (e.g., the patient) over a period of time that the user utilizes the continuous analyte monitoring system 104 and the application 106. For example, analyte data for a user who has used the continuous analyte monitoring system 104 and the application 106 for a period of five years to manage their condition may have time series analyte data for the user maintained in the user profile 118 over the five-year period.

Further, data stored in the user profile 118 may provide time series data collected over the lifetime of the user. For example, the data may include information for the user that indicates physiological states of the user, glucose levels of the user, lactate levels of the user, ketone levels of user, states/conditions of one or more organs of the user, habits of the user (e.g., alcohol consumption, activity levels, food consumption, etc.), medications prescribed, etc., throughout the lifetime of the user.

The user profile 118 may be dynamic because at least part of the information that is stored in the user profile 118 may be revised or updated over time or new information may be added to user profile 118 by the analytics engines 114 or the application 106. Accordingly, the information in the user profile 118 stored in the user database 110 provides an up-to-date repository of information for the user.

The user database 110 may include a storage server that operates, for example, in a public or private cloud. The user database 110 may be implemented as any type of datastore, such as relational databases, non-relational databases, key-value datastores, file systems including hierarchical file systems, and the like. In some exemplary implementations, the user database 110 is distributed. For example, the user database 110 may include persistent storage devices, which are distributed. Furthermore, the user database 110 may be replicated so that the storage devices are geographically dispersed.

The user database 110 includes the user profiles 118 for multiple users, including users who similarly interact or have interacted in the past with the application 106 on their own devices. The user profiles stored in the user database 110 are accessible not only to the application 106, but to the analytics engines 114, as well. The user profiles in the user database 110 may be accessible to the application 106 and the analytics engines 114 over one or more networks (not shown). As described above, the analytics engines 114 can fetch inputs 128 from a user's profile 118 stored in the user database 110 and compute one or more metrics 130, which can then be stored as application data 126 in the user's profile 118.

The user profiles 118 stored in the user database 110 may also be stored in the historical records database 112. The user profiles 118 stored in the historical records database 112 may provide a repository of up-to-date information and historical information (historical event information 132) for each user of the application 106. Thus, the historical records database 112 essentially provides all data related to each user of the application 106, where data is stored using timestamps. The timestamp for any piece of information stored in the historical records database 112 may identify, for example, when the piece of information was obtained or updated.

Further, the historical records database 112 may include data for one or more patients who are not users of the continuous analyte monitoring system 104 or the application 106. For example, the historical records database 112 may include information (e.g., user profiles) for one or more patients analyzed by, for example, a healthcare physician (or other known method), who may or may not be diagnosed with diabetes. Data stored in the historical records database 112 may be referred to herein as population data.

Although depicted as separate databases for conceptual clarity, the user database 110 and the historical records database 112 may operate as a single database. The single database may be a storage server that operates in a public or private cloud.

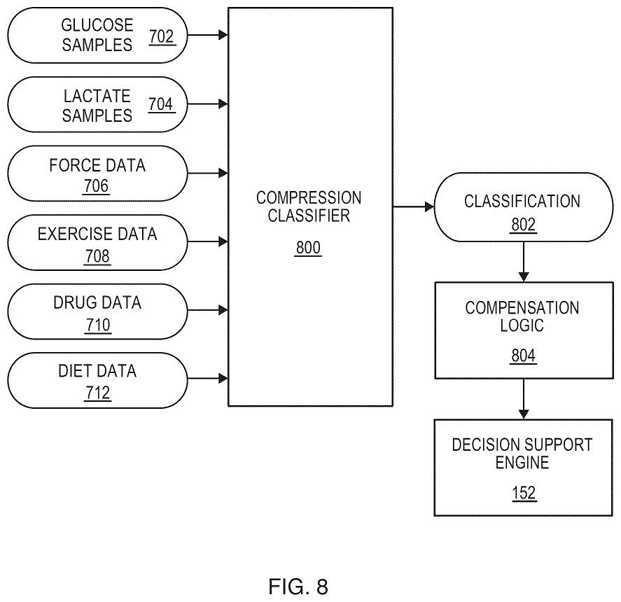

The training server system 140 may train the one or more machine learning models using training data, which may include data (e.g., from user profiles) for one or more patients (e.g., users or non-users of the continuous analyte monitoring system 104 or the application 106, e.g., diabetic patients). The training data may be stored in the historical records database 112 and may be accessible to the training server system 140 over one or more networks (not shown) for training the machine learning models. In particular, the training server system 140 may train a machine learning model to classify compression events as described below with respect to FIG. 8.

The training data may include a dataset that has been featurized and labeled. For example, the dataset may include a plurality of data records, each including information from a different user profile stored in user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning models.

The one or more models are then trained by the training server system 140 using the featurized and labeled training data. In particular, the features of each data record may be used as input into the machine learning models, and the generated output may be compared to labels associated with the corresponding data record. In certain embodiments, the models may compute a loss based on the difference between the generated output and the provided labels. This loss is then used to modify the internal parameters or weights of the model. By iteratively processing each data record for each historical patient, the models may be iteratively refined to generate predictions distinguishing between sensor failure and compression events.

As illustrated in FIG. 1, the training server system 140 deploys these trained models to the analytics engines 114 for use during runtime. For example, the compression event detection module 154 may process signals from the continuous analyte monitoring system and output a determination to determine whether a compression event has occurred and possibly a type of compression event (e.g., shown as output 144 in FIG. 1). In certain embodiments, the output 144 generated by the compression event detection module 154 may be stored in the user profile 118.

FIG. 2 is a diagram 200 conceptually illustrating an example continuous analyte monitoring system 104 including example continuous analyte sensors with sensor electronics, in accordance with certain aspects of the present disclosure. For example, the system 104 may continuously monitor one or more analytes of a user, in accordance with certain aspects of the present disclosure.

The continuous analyte monitoring system 104 includes sensor electronics module 204 and one or more continuous analyte sensors 202 (individually referred to herein as continuous analyte sensor 202 and collectively referred to herein as continuous analyte sensors 202). The sensor electronics module 204 may be in wired or wireless communication (e.g., directly or indirectly) with one or more of display devices 210, 220, 230, and 240. The sensor electronics module 204 may also be in wired or wireless communication (e.g., directly or indirectly) with one or more medical devices, such as medical devices 208 (individually referred to herein as medical device 208 and collectively referred to herein as medical devices 208), or one or more other non-analyte sensors 206 (individually referred to herein as non-analyte sensor 206 and collectively referred to herein as non-analyte sensor 206).

A continuous analyte sensor 202 may include one or more sensors for detecting or measuring analytes. A continuous analyte sensor 202 may be a multi-analyte sensor that continuously measures two or more analytes (e.g., glucose, lactate, potassium, ketone, etc.), or a single analyte sensor that continuously measures a single analyte (e.g., where one continuous analyte sensor 202 is used for measuring glucose and then a second continuous analyte sensor 202 used for measuring lactate, etc.). The continuous analyte sensor 202 may be a non-invasive device, a subcutaneous device, a transcutaneous device, a transdermal device, or an intravascular device. The continuous analyte sensor 202 may continuously measure analyte levels of a user using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The continuous analyte sensor 202 may provide a data stream indicative of the concentration of one or more analytes in the user. The data stream may include raw data signals, which are then converted into a calibrated or filtered data stream, e.g., by the analytics engines 114 as described above, and are used to provide estimated analyte values to the user. The raw data signals may further be analyzed by the compression event detection module 154 to determine whether a compression event is indicated and possibly a type of compression event.

The continuous analyte sensor 202 may be a multi-analyte sensor that continuously measures multiple analytes in a user's body. For example, the continuous multi-analyte sensor 202 may be a single sensor that measures glucose, lactate, ketones, glycerol, potassium (e.g., hyperkalemia), sodium, CO2 or anion-gap, or similar analytes in the user's body.

The sensor electronics module 204 includes electronic circuitry for measuring and processing the continuous analyte sensor data. The sensor electronics module 204 can be physically connected to the continuous analyte sensors 202 and can be integral with (non-releasably attached to) or releasably attachable to the continuous analyte sensors 202. The sensor electronics module 204 may include hardware, firmware, or software that enable measurement of levels of analytes via the continuous analyte sensors 202. For example, the sensor electronics module 204 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, or a processor.

The display devices 210, 220, 230, or 240 may display displayable sensor data, including analyte data, which may be transmitted by the sensor electronics module 204. The sensor electronics module 204 may transmit raw sensor data that is converted to displayable sensor data via one or more of the display devices 210, 220, 230, and 240. The sensor electronics module 204 may convert raw sensor data to displayable sensor data and transmit the displayable sensor data to one or more of the display devices 210, 220, 230, or 240. Each of the display devices 210, 220, 230, or 240 may include a display such as a touchscreen display 212, 222, 232, or 242 for displaying sensor data to a user or for receiving inputs from the user. For example, a graphical user interface (GUI) may be presented to the user for such purposes. The display devices 210, 220, 230, and 240 may include other types of user interfaces such as a voice user interface instead of, or in addition to, a touchscreen display for communicating sensor data to the user of the display device or for receiving user inputs. The display devices 210, 220, 230, and 240 may be examples of display device 107 illustrated in FIG. 1 used to display sensor data to a user of the system of FIG. 1 or to receive input from the user.

The display devices 210, 220, 230, and 240 may display or otherwise communicate the sensor data as it is communicated from the sensor electronics module 204 (e.g., in a customized data package that is transmitted to the display devices 210, 220, 230, and 240 based on their respective preferences), without any additional prospective processing required for calibration and real-time display of the sensor data.

The display devices 210 may include a custom display device specially designed for displaying certain types of displayable sensor data for analyte data received from the sensor electronics module 204. The display device 220 may be a smartphone or a mobile phone using a commercially available operating system (OS) and may display a graphical representation of the continuous sensor data (e.g., including current and historic data). The display device 230 may include a tablet, and the display device 240 may include a smart watch. The medical device 208 may include an insulin delivery device or a blood glucose meter. The display devices 210, 220, 230, and 240 and the medical device 208 may include a desktop or laptop computer (not shown).

Because different display devices provide different user interfaces, content of the data packages (e.g., amount, format, or type of data to be displayed, alarms, and the like) can be customized (e.g., programmed differently by the manufacture or by an end user) for each particular display device. Accordingly, different display devices can be in direct wireless communication with the sensor electronics module 204 (e.g., such as an on-skin sensor electronics module 204 that is physically connected to continuous analyte sensors 202) during a sensor session to enable a plurality of different types or levels of display or functionality for the displayable sensor information.

As mentioned, the sensor electronics module 204 may be in communication with a medical device 208. The medical device 208 may be a passive device. For example, the medical device 208 may be an insulin pump for administering insulin to a user. For a variety of reasons, it may be desirable for such an insulin pump to receive and track analyte values, e.g., glucose values, transmitted from the continuous analyte monitoring systems 104, where the continuous analyte sensor 202 includes at least a glucose sensor.

Further, as mentioned, the sensor electronics module 204 may also be in communication with other non-analyte sensors 206. The non-analyte sensors 206 may include, but are not limited to, a temperature sensor, a force sensor, oxygen sensor, an altimeter sensor, an accelerometer sensor, a gyrometer sensor, a global positioning system (GPS) sensor, a respiratory sensor, electromyogram (EMG) sensor, a galvanic skin response (GSR) sensor, an impedance sensor, an electrocardiogram sensor, a sweat sensor, etc. The non-analyte sensors 206 may also include monitors such as heart rate monitors, blood pressure monitors, pulse oximeters, caloric intake monitors, and medicament delivery devices. One or more of these non-analyte sensors 206 may provide data to the compression event detection module 154 described further below.

The non-analyte sensors 206 may be combined in any other configuration, such as, for example, combined with one or more continuous analyte sensors 202. As an illustrative example, a non-analyte sensor, e.g., a temperature sensor, may be combined with a continuous glucose analyte sensor 202 configured to sense glucose to form a glucose/temperature sensor used to transmit sensor data to the sensor electronics module 204 using common communication circuitry. As another illustrative example, a non-analyte sensor, e.g., a temperature sensor, may be combined with a multi-analyte sensor 202 that measures glucose and, e.g., lactate to form a glucose/lactate/temperature sensor used to transmit sensor data to the sensor electronics module 204 using common communication circuitry.

One or more of the continuous analyte monitoring systems 104, the plurality of display devices, the medical devices 208, or the non-analyte sensors 206 may communicate together wirelessly using one of a variety of wireless communication technologies (e.g., Wi-Fi, Bluetooth, Near Field Communication (NFC), cellular, etc.). A wireless access point (WAP) may be used to couple one or more of the continuous analyte monitoring system 104, the plurality of display devices, the medical devices 208, or the non-analyte sensors 206 to one another. For example, the WAP may provide Wi-Fi, Bluetooth, or cellular connectivity among these devices. NFC may also be used among the devices depicted in the diagram 200 of FIG. 2.

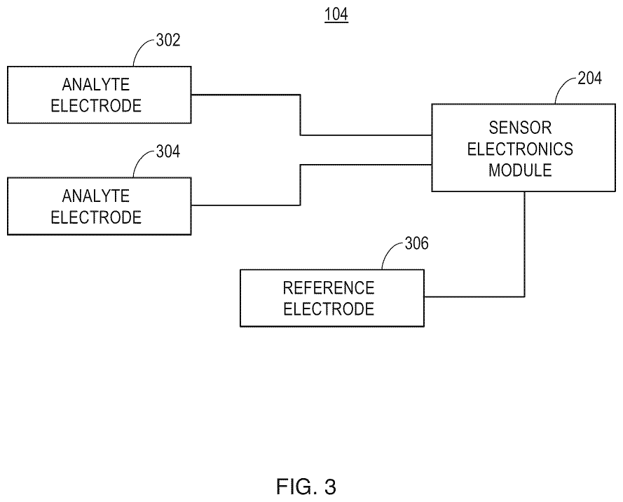

FIG. 3 illustrates an example continuous analyte monitoring system 104, according to some embodiments disclosed herein. The continuous analyte monitoring system 104 includes the sensor electronics module 204 as well one or more working electrodes that each operate as an analyte sensor. As seen in FIG. 3, the continuous analyte monitoring system 104 includes one or more analyte electrodes 302 and 304 (which may also be referred to as “working electrodes”) and a reference electrode 306. The continuous analyte monitoring system 104 may include any suitable number of analyte electrodes (e.g., one or more analyte electrodes). Each of the electrodes may include a membrane over its surface. The electrodes may be at least partially covered by the same membrane. Alternatively, one or more of the electrodes may not be covered by a membrane as in a bare electrode. The electrodes may be electrically coupled to the sensor electronics module 204 (e.g., the potentiostat and the processor module of the sensor electronics module 204).

The electrodes may be formed of any suitable materials and by any suitable methods. For example, the electrodes may be formed of one or more noble metals, such as platinum, palladium, rhodium, iridium, ruthenium, or platinum/iridium. In some embodiments, the electrodes are carbon-based, and include carbon, carbon/ruthenium, doped diamond, carbon nanotube, graphene, graphite, amorphous carbon, or carbon fiber. In certain embodiments, the electrodes are formed of graphite, gold, conductive polymer, indium tin oxide, or the like. The reference electrode 306 may include silver, silver/silver chloride, or iridium oxide and may be kept currentless. Generally, suitable methods for forming the electrodes include roll-to-roll techniques, screen printing, microfabrication techniques, such as physical vapor deposition, chemical vapor deposition, electrodeposition, lithography, and/or etching techniques. Other methods, including spray deposition or dip-coating, are also contemplated.

The continuous analyte monitoring system 104 may be positioned on a body of a user (e.g., user 102) by inserting some or all of the working electrodes into the body. The working electrodes may be inserted into an adequate insertion site, such as an abdomen or an arm of the user 102, where the working electrodes may be in contact with the blood or interstitial fluid of the user 102.

Different electrodes may detect different analytes. For example, each of the electrodes may include an active surface to facilitate electrochemical sensing of desired analytes. The active surface of each electrode may be voxelated, or partitioned into discrete sections (e.g., cubic sections). Different biorecognition elements (e.g., enzymes, antibodies, aptamers, double-stranded deoxyribonucleic acid (DNA), single-stranded DNA, ribonucleic acid (RNA), oligonucleotides, proteins, cells, microbes, ion-selective materials, etc.), each specific to a different analyte, may be deposited and immobilized on each active surface of each electrode. For example, both glucose oxidase and lactate or uric acid oxidase (and/or other analyte-specific enzymes) may be deposited on the active surface of one electrode. Different enzymes may be immobilized on each voxel of each electrode. In some embodiments, only one type of enzyme is deposited on each voxel of each electrode. The enzymes may be immobilized via adsorption, entrapment, cross-linking, covalent bonding, or any other suitable immobilization methods.

Each of the electrodes includes enzymes for one specific analyte, while different electrodes may include enzymes for different analytes. In certain embodiments, some of the electrodes include two or more enzymes, such as four or more enzymes, which together enable sensing of a single analyte. For example, where an electrode is configured to sense creatinine, the electrode may include four or more enzymes specific for creatinine. An example of a 1-enzyme sensor electrode may include a lactate-specific electrode, and an example of a 2-enzyme sensor electrode may include a ketone-specific electrode. In the example of FIG. 3, the analyte electrode 302 may be used to measure a level of a first analyte (e.g., glucose). The analyte electrode 304 may be used to measure a level of a second analyte (e.g., lactate). The reference electrode 306 may have a known electrical potential that may serve as a reference potential when measuring or determining the electrical potential of the other electrodes.

During sensing, the deposited enzymes are utilized to convert a respective analyte to an intermediary product (e.g., hydrogen peroxide), which is then oxidized at the surface of the electrodes. The resulting current flow, which is measured by the potentiostat of the sensor electronics module 204 or an ammeter in communication with the potentiostat, is proportional to the analyte concentration. Examples of suitable enzymes include glucose oxidase for sensing glucose species, lactate oxidase for sensing lactate species, lactose oxidase for sensing lactose species, glutamate oxidase for glutamate species, and the like. In addition to enzymes, the active surfaces may further include immobilized redox mediators (e.g., relays) (not shown), which are small electroactive molecules for shuttling electrons between the enzymes and the electrodes. In other embodiments, active surfaces may further include enzyme co-factors, which are compounds used by the enzyme to convert a substrate to a product. In some embodiments, the enzymes are immobilized exclusively over the skive regions of electrodes to minimize or avoid cross-talk between different analytes. In other words, an area of active surface over each of the electrodes may be less than a geometric surface area of the respective working electrode. Maintaining a potential bias at the electrodes may facilitate a near-zero peroxide efflux from the skive region with active consumption of the hydrogen peroxide intermediary.

The continuous analyte monitoring system 104 may use one or more of the signal streams from the analyte electrode 302 and/or the analyte electrode 304 to determine if one of the analyte electrodes 302 or 304 are defective or have detached from the user. In some embodiments, the continuous analyte monitoring system 104 generates a first analyte signal stream using the analyte electrode 302 and a second analyte signal stream using the analyte electrode 304. For example, the analyte electrodes 302 and 304 may generate current flows when particular analytes interact with the surfaces of the analyte electrodes 302 and 304. The potentiostat of the sensor electronics module 204 may measure and report these current flows to the processor of the processor module 214. The processor may then generate the analyte signal streams that represent the measured current flows.

In the following embodiments, a continuous analyte monitoring system 104 including analyte electrodes 302 and 304 is discussed with the understanding that other types of analyte sensors may be used in a like manner and would likewise benefit from sensing or mitigating compression events. Such other types of analyte sensors may include those emitting and sensing light, such as a blood oxygen (SpO₂) sensor using light emitting diodes (LED) and photodiodes.

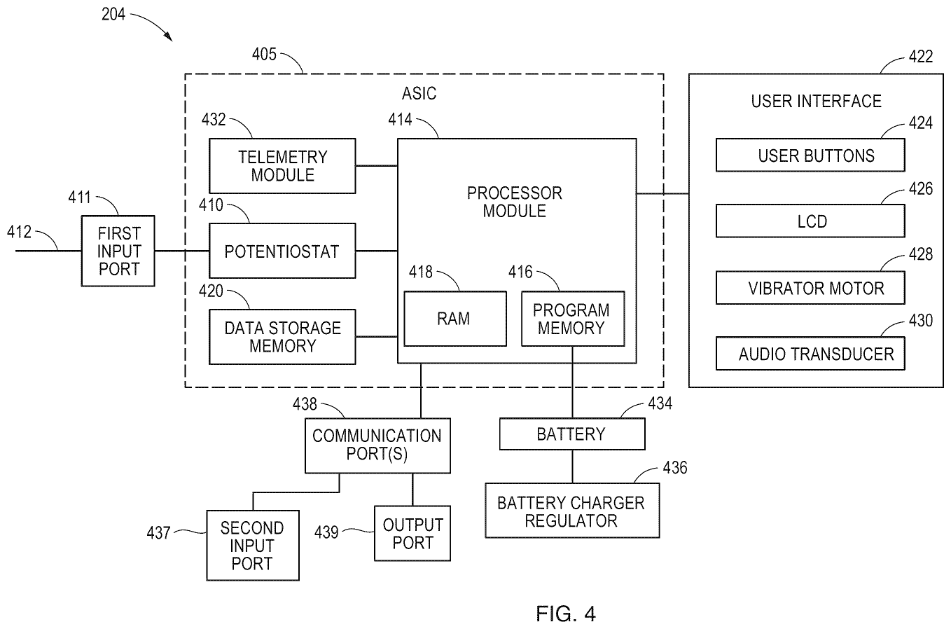

FIG. 4 depicts an example implementation of the sensor electronics module 204, in accordance with some example implementations. The sensor electronics module 204 may include sensor electronics that process sensor information, such as sensor data, and generate transformed sensor data and displayable sensor information, e.g., via a processor module. For example, the processor module may transform sensor data into one or more of the following: filtered sensor data (e.g., one or more filtered analyte concentration values), raw sensor data, calibrated sensor data (e.g., one or more calibrated analyte concentration values), rate of change information, trend information, rate of acceleration/deceleration information, sensor diagnostic information, location information, alarm/alert information, calibration information such as may be determined by factory calibration algorithms as disclosed herein, smoothing or filtering algorithms of sensor data, or the like.

A processor module 414 achieves a substantial portion, if not all, of the data processing, including data processing pertaining to factory calibration. The processor module 414 may include a hardware processor or processor circuitry. The processor module 414 may be integral to the sensor electronics module 204 or may be located remotely, such as in one or more of devices 210, 220, 230, 240 or in a cloud computing platform. The processor module 414 may include smaller subcomponents or submodules. For example, the processor module 414 may include an alert module (not shown) or prediction module (not shown), or any other suitable module that may be utilized to efficiently process data. When the processor module 414 includes submodules, the submodules may be located within the processor module 414, including within the sensor electronics module 204 or other associated devices (e.g., 210, 220, 230, 240). For example, the processor module 414 may be located at least partially within a cloud-based analyte processor or elsewhere in a network.

The processor module 414 may calibrate the sensor data, and the data storage memory 420 may store the calibrated sensor data points as transformed sensor data. Moreover, the processor module 414 may wirelessly receive calibration information from a display device, such as devices 210, 220, 230, 240, to enable calibration of the sensor data from the sensor 202. Furthermore, the processor module 414 may perform additional algorithmic processing on the sensor data (e.g., calibrated or filtered data or other sensor information), and the data storage memory 420 may store the transformed sensor data or sensor diagnostic information of the algorithms. The processor module 414 may store and use calibration information determined from a factory calibration, as described below.

The sensor electronics module 204 may include an application-specific integrated circuit (ASIC) 405 coupled to a user interface 422. The ASIC 405 may further include a potentiostat 410, a telemetry module 432 for transmitting data from the sensor electronics module 204 to one or more devices, such as devices 210, 220, 230, 240, or other components for signal processing and data storage (e.g., processor module 414 and data storage memory 420). Although FIG. 4 depicts ASIC 405, other types of circuitry may be used as well, including field programmable gate arrays (FPGA), one or more microprocessors that provide some (if not all of) the processing performed by the sensor electronics module 204, analog circuitry, digital circuitry, or a combination thereof.

In the example of FIG. 4, through a first input port 411 for sensor data the potentiostat 410 is coupled to a continuous analyte sensor 202, such as a glucose sensor to generate sensor data from the analyte. The potentiostat 410 may also provide via data line 412 a voltage to the continuous analyte sensor 202 to bias the sensor for measurement of a value (e.g., a current and the like) indicative of the analyte concentration in a host (also referred to as the analog portion of the sensor). The potentiostat 410 may have one or more channels depending on the number of working electrodes at the continuous analyte sensor 202.

The potentiostat 410 may include a resistor that translates a current value from the sensor 202 into a voltage value, or a current-to-frequency converter (not shown) may also integrate continuously a measured current value from the sensor 202 using, for example, a charge-counting device. An analog-to-digital converter (not shown) may digitize the analog signal from the sensor 202 into so-called “counts” to allow processing by the processor module 414. The resulting counts may be directly related to the current measured by the potentiostat 410, which may be directly related to an analyte level, such as a glucose level, in the host.

The telemetry module 432 may be operably connected to the processor module 414 and may provide the hardware, firmware, or software that enable wireless communication between the sensor electronics module 204 and one or more other devices, such as display devices, processors, network access devices, and the like. A variety of wireless radio technologies that can be implemented in the telemetry module 432 include Bluetooth, Bluetooth Low-Energy, ANT, ANT+, ZigBee, IEEE 802.11, IEEE 802.16, cellular radio access technologies, radio frequency (RF), infrared (IR), paging network communication, magnetic induction, satellite data communication, spread spectrum communication, frequency hopping communication, near field communications, or the like. The telemetry module 432 may include a Bluetooth chip, although Bluetooth technology may also be implemented in a combination of the telemetry module 432 and the processor module 414.

The processor module 414 may control the processing performed by the sensor electronics module 204. For example, the processor module 414 may process data (e.g., counts), from the sensor, filter the data, calibrate the data, perform fail-safe checking, or the like.

The processor module 414 may include a digital filter, such as for example an infinite impulse response (IIR) or a finite impulse response (FIR) filter. This digital filter may smooth a raw data stream received from the sensor 202. Generally, the digital filters are programmed to filter data sampled at a predetermined time interval (also referred to as a sample rate). When the potentiostat 410 measures the analyte (e.g., glucose or the like) at discrete time intervals, these time intervals determine the sampling rate of the digital filter. The potentiostat 410 may measure continuously the analyte, for example, using a current-to-frequency converter. In these current-to-frequency converter implementations, the processor module 414 may be programmed to request, at predetermined time intervals (acquisition time), digital values from the integrator of the current-to-frequency converter. These digital values obtained by the processor module 414 from the integrator may be averaged over the acquisition time due to the continuity of the current measurement. As such, the acquisition time may be determined by the sampling rate of the digital filter.

The processor module 414 may further include a data generator (not shown) that generates data packages for transmission to devices, such as the display devices 210, 220, 230, 240. Furthermore, the processor module 414 may generate data packets for transmission to these outside sources via the telemetry module 432. In some example implementations, the data packages may, as noted, be customizable for each display device, or may include any available data, such as a time stamp, displayable sensor information, transformed sensor data, an identifier code for the sensor or sensor electronics module 204, raw data, filtered data, calibrated data, rate of change information, trend information, error detection or correction, or the like.

The processor module 414 may also include a program memory 416 and other memory 418. The processor module 414 may be coupled to a communications interface, such as a communication port 438, and a source of power, such as a battery 434. Moreover, the battery 434 may be further coupled to a battery charger or regulator 436 to provide power to sensor electronics module 204 or charge the battery 434.

The program memory 416 may be implemented as a semi-static memory for storing data, such as an identifier for a coupled sensor 202 (e.g., a sensor identifier (ID)) and for storing code (also referred to as program code) to configure the ASIC 405 to perform one or more of the operations/functions described herein. For example, the program code may configure processor module 414 to process data streams or counts, filter, perform the calibration methods described below, perform fail-safe checking, and the like.

The memory 418 may also be used to store information. For example, the processor module 414 including memory 418 may be used as the system's cache memory, where temporary storage is provided for recent sensor data received from the sensor. In some example implementations, the memory may include memory storage components, such as read-only memory (ROM), random-access memory (RAM), dynamic-RAM, static-RAM, non-static RAM, easily erasable programmable read only memory (EEPROM), rewritable ROMs, flash memory, and the like.

The data storage memory 420 may be coupled to the processor module 414 and may store a variety of sensor information. In some example implementations, the data storage memory 420 stores one or more days of continuous analyte sensor data. For example, the data storage memory may store 1, 2, 3, 4, 5, 6, 7, 8, 9, 10, 11, 12, 13, 14, 15, or 30 days (or more days) of continuous analyte sensor data received from the sensor 202. The stored sensor information may include one or more of the following: a time stamp, raw sensor data (one or more raw analyte concentration values), calibrated data, filtered data, transformed sensor data, or any other displayable sensor information, calibration information (e.g., reference BG values or prior calibration information such as from factory calibration), sensor diagnostic information, and the like.

The user interface 422 may include a variety of interfaces, such as one or more buttons 424, a liquid crystal display (LCD) 426, a vibrator 428, an audio transducer (e.g., speaker) 430, a backlight (not shown), or the like. The components that include the user interface 422 may provide controls to interact with the user (e.g., the host). One or more buttons 424 may allow, for example, toggle, menu selection, option selection, status selection, yes/no response to on-screen questions, a “turn off” function (e.g., for an alarm), an “acknowledged” function (e.g., for an alarm), a reset, or the like. The LCD 426 may provide the user with, for example, visual data output. The audio transducer 430 (e.g., speaker) may provide audible signals in response to triggering of certain alerts, such as present or predicted hyperglycemic and hypoglycemic conditions. Audible signals may be differentiated by tone, volume, duty cycle, pattern, duration, or the like. The audible signal may be silenced (e.g., acknowledged or turned off) by pressing one or more buttons 424 on the sensor electronics module 204 or by signaling the sensor electronics module 204 using a button or selection on a display device (e.g., key fob, cell phone, or the like).

Although audio and vibratory alarms are described with respect to FIG. 4, other alarming mechanisms may be used as well. For example, in some example implementations, a tactile alarm is provided including a poking mechanism that “pokes” or physically contacts the patient in response to one or more alarm conditions.

The battery 434 may be operatively connected to the processor module 414 (and possibly other components of the sensor electronics module 204) and provide the necessary power for the sensor electronics module 204. The battery may be a lithium manganese dioxide battery, however any appropriately sized and powered battery can be used (e.g., AAA, nickel-cadmium, zinc-carbon, alkaline, lithium, nickel-metal hydride, lithium-ion, zinc-air, zinc-mercury oxide, silver-zinc, or hermetically-sealed). The battery may be rechargeable. Multiple batteries can be used to power the system. In yet other implementations, the receiver can be transcutaneously powered via an inductive coupling, for example.

A battery charger or regulator 436 may receive energy from an internal or external charger. The battery regulator (or balancer) 436 regulates the recharging process by bleeding off excess charge current to allow all cells or batteries in the sensor electronics module 204 to be fully charged without overcharging other cells or batteries. The battery 434 (or batteries) may be charged via an inductive or wireless charging pad, although any other charging or power mechanism may be used as well.

One or more communication ports 438, also referred to as external connectors, may be provided to allow communication with other devices, for example a PC communication (com) port can be provided to enable communication with systems that are separate from, or integral with, the sensor electronics module 204. The communication port, for example, may include a serial (e.g., universal serial bus or “USB”) communication port, and allow for communicating with another computer system (e.g., PC, personal digital assistant or “PDA,” server, or the like). The sensor electronics module 204 may transmit historical data to a PC or other computing device (e.g., an analyte processor as disclosed herein) for retrospective analysis by a patient or physician. As another example of data transmission, factory information may also be sent to the algorithm from the sensor or from a cloud data source.

The one or more communication ports 438 may further include a second input port 437 in which calibration data may be received, and an output port 439 which may be employed to transmit calibrated data, or data to be calibrated, to a receiver or mobile device. FIG. 4 illustrates these aspects schematically. It will be understood that the ports may be separated physically, but in alternative implementations a single communication port may provide the functions of both the second input port and the output port.

In some continuous analyte sensor systems, an on-skin portion of the sensor electronics may be simplified to minimize complexity or size of on-skin electronics, for example, providing only raw, calibrated, or filtered data to a display device configured to run calibration and other algorithms required for displaying the sensor data. However, the sensor electronics module 204 (e.g., via processor module 414) may be implemented to execute prospective algorithms used to generate transformed sensor data or displayable sensor information, including, for example, algorithms that: evaluate a clinical acceptability of reference or sensor data, evaluate calibration data for best calibration based on inclusion criteria, evaluate a quality of the calibration, compare estimated analyte values with time corresponding measured analyte values, analyze a variation of estimated analyte values, evaluate a stability of the sensor or sensor data, detect signal artifacts (noise), replace signal artifacts, determine a rate of change or trend of the sensor data, perform dynamic and intelligent analyte value estimation, perform diagnostics on the sensor or sensor data, set modes of operation, evaluate the data for aberrancies, or the like.

Although separate data storage and program memories are shown in FIG. 4, a variety of configurations may be used as well. For example, one or more memories may be used to provide storage space to support data processing and storage requirements at sensor electronics module 204.

### Compression Event Detection

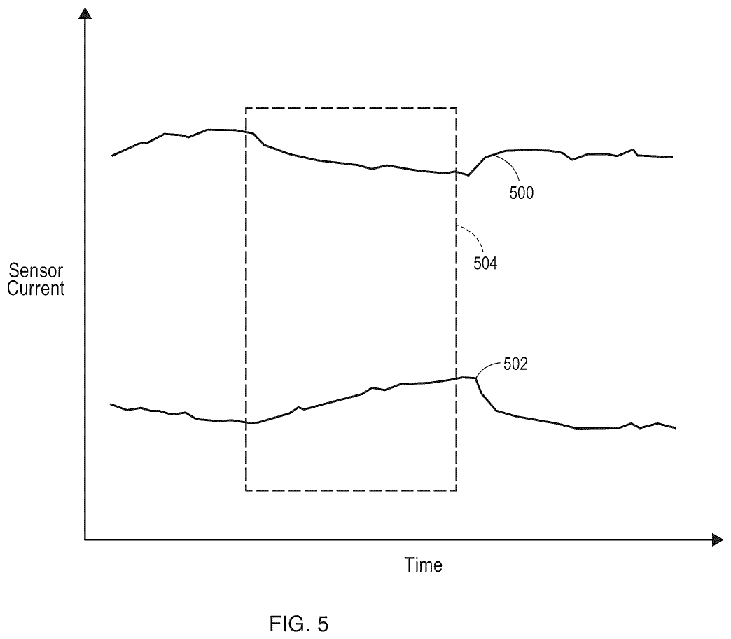

FIGS. 5 through 10 illustrate systems and methods for detecting compression events and, if possible, compensating data output by the continuous analyte monitoring system 104.

FIG. 5 illustrates a plot 500 of the output of a glucose sensor and a plot 502 of the output of a lactate sensor such as may be incorporated into the continuous analyte sensor 202. As shown, during a compression event 504, the output of the glucose sensor will fall while the output of the lactate sensor will rise. This inverse movement may result from a lack of oxygen triggering anaerobic respiration or some other physiological process. In the absence of a compression event 504, the output of the glucose sensor and output of the lactate sensor will move in correlation over the long term, with some lag.

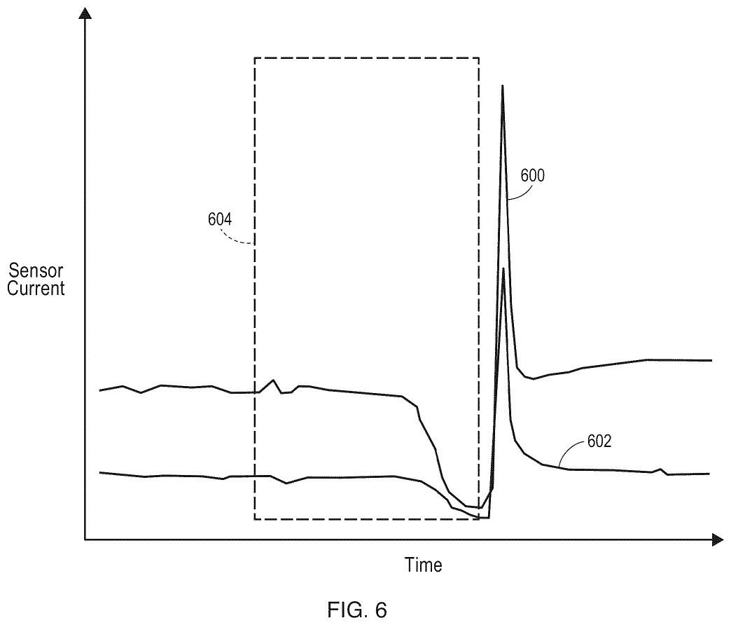

FIG. 6 illustrates a plot 600 of the output of a glucose sensor and a plot 602 of the output of a lactate sensor such as may be incorporated into the continuous analyte sensor 202. During an acute compression event 604, the outputs of the glucose sensor and lactate sensor will vary drastically and in correlation. As is apparent, the outputs of the glucose sensor and lactate sensor have a “biphasic” response to an acute compression event: a steep drop during the compression event and a sharp spike following the compression event.

As is apparent from FIGS. 5 and 6, the outputs of the glucose sensor and lactate sensor during and immediately after a compression event 504, 604 can exhibit clear patterns of inverse correlation or a correlated biphasic fluctuation. These patterns may be used, possibly with other information, to compensate for compression events by either (a) adjusting sampled values or (b) suppressing use of sampled values (“blanking”).

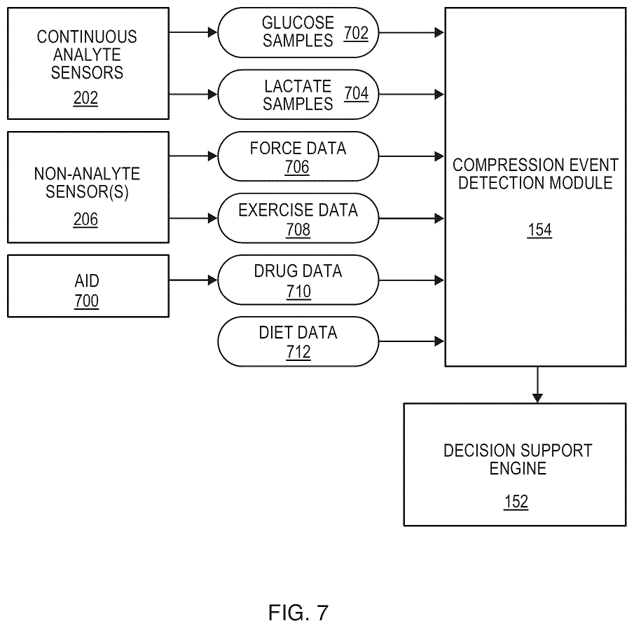

For example, referring to FIG. 7, the compression event detection module 154 may receive inputs from one or more continuous analyte sensors 202, one or more non-analyte sensors 206, and possibly from an automatic insulin device (AID) 700, such as an insulin pump supplying subcutaneous insulin to the user of the continuous analyte monitoring system 104.

For example, the compression event detection module 154 may receive glucose samples 702 and lactate samples 704 from the continuous analyte sensor 202. The glucose samples 702 and lactate samples 704 may be samples of the current output by analyte electrodes 302, 304 whether in raw form or following one or more pre-processing steps such as amplification or filtering.

The compression event detection module 154 may receive a force data 706 output from the non-analyte sensors 206, such as from a force sensor implemented as a load cell, pressure-sensitive switch, force-sensitive resistor, or any other device capable of sensing force exerted thereon. The force sensor may be, for example, mounted in or on a housing of the continuous analyte monitoring system 104 such that force exerted on the force sensor correlates to force exerted on the analyte electrodes 302, 304 and the tissue surrounding the analyte electrodes 302, 304.

The compression event detection module 154 may receive exercise data 708 from the non-analyte sensors 206. For example, the non-analyte sensors 206 may include an accelerometer and the exercise data 708 may be the output of the accelerometer or a representation of the output of the accelerometer, such as a step count, estimated distance walked, run, or swum. The non-analyte sensors 206 may include a heart rate monitor and the exercise data 706 may be the output of the heart rate monitor or a representation of the output of the accelerometer, such as average heart rate for a time period, maximum heart rate for a time period, amount of time spent above a threshold heart rate or in a heart rate range, an estimate of calories burned based on measurements of heart rate, or other data derived from the output of the heart rate monitor. The exercise data 708 may include data facilitating interpretation of the output of an accelerometer or heart rate monitor, such as weight, biological gender, resting heart rate, or other data.

The AID 700, the user, or some other individual or device may supply drug data 710 to the compression event detection module 154. The drug data 710 may include an amount of insulin supplied subcutaneously to the user. The amount may be represented as a volume injected and a time of injection. The amount may be represented as a rate of insulin delivery by the AID 700 during a time period, such as an hour, 15 minutes, 5 minutes, or some other interval.

The compression event detection module 154 may receive diet data 712. The diet data 712 may include data describing calories, protein, carbohydrates, sodium or other macro- and micronutrients by the user. The diet data 712 may be input by the user or other individual into an application on a display device 107, 210, 220, 230, 240 or some other device.

The compression event detection module 154 receives some or all of the above-referenced data and provides an output to the decision support engine 152. The output may include raw data from the analyte electrodes 302, 304 without modification, or without modification compensating for a compression event. The output may include adjusted data obtained by adjusting the raw data to compensate for a compression event. The output may be modified to adjust data corresponding to a compression event or include blanked data for the compression event. Blanked data may include data that indicates that no glucose and/or lactate measurement are available for a time period, such as for a sampling period or block of sampling periods. For example, the sampling period may be every 30 seconds or some other time interval.

The decision support engine 152 will then process the output as described above in order to provide alerts regarding hyper- or hypo-glycemic events and/or providing recommendations with respect to nutrition and administration of insulin.

Referring to FIG. 8, the compression event detection module 154 may include a compression classifier 800. The compression classifier 800 may receive some or all of the data described above as being input to the compression event detection module 154. In some embodiments, the compression classifier 800 receives only the glucose samples 702 and lactate samples 704. In other embodiments, the compression classifier 800 receives only the glucose samples 702 and lactate samples 704 along with force data 706.

The compression classifier 800 may be a machine learning model such as a logistic regression machine learning model, decision tree machine learning model, Bayesian machine learning model, neural network, deep neural network, convolution neural network, or any other type of machine learning model.

The compression classifier 800 may be trained with training data entries. Each training data entry may include, as a desired output, a classification, e.g., a first value indicating no compression event, a second value indicating a compression event that is compensatable, and a third value indicating an acute compression event that should result in blanking. As used herein “compensatable” may be understood as meaning that glucose samples can be adjusted to compensate for a compression event, such as using an assumed inverse correspondence with lactate samples. Each training data entry may include, as inputs, some or all of the glucose samples 702, lactate samples 704, force data 706, exercise data 708, drug data 710, and diet data 712 measured for a patient for a time period that either does not include a compression event or for a time period including a compression event, whether acute or not. The inputs may include data for just the time period during which the compression event occurred or may include data for a time period extending after the compression event and possibly before and after the compression event.

The data input to the compression classifier 800 may include data derived from some or all of the data input to the compression event detection module 154. For example, for data represented as a series of samples or measurement, the data input to the compression classifier 800 may be a smoothed version of such data, such as using an exponential smoothing function or other type of smoothing function. The data input to the compression classifier 800 may include features extracted from the data, such as mean, variability, outlier count (e.g., Z-score<−2), maximum value, minimum value, 25^{th} percentile, 75^{th} percentile, standard deviation, or other statistical characterization.

The compression classifier 800 may be trained using the training data entries. For example, the inputs of each training data entry may be processed using the compression classifier 800 to obtain an output that is compared to the desired output of the training data entry. A training algorithm may then update parameters of the compression classifier 800 based on the comparison. The compression classifier 800 is therefore trained to output a classification 802 for a given set of inputs indicating whether the inputs correspond to a compensatable compression event, an acute compression event, or the absence of a compression event.

The classification 802 and other data, such as the glucose samples 702 and lactate samples 704 may be input to compensation logic 804. The compensation logic may do nothing where the classification 802 indicates no compression event. The compensation logic may output blanked data or suppress output of the glucose samples 702 and lactate samples 704 where the classification 802 indicates an acute compression event. The samples blanked may include all samples within the time window that were processed by the compression classifier 800 to obtain the classification. The samples blanked may include all samples that are below a threshold, such as a threshold that corresponds to samples that would otherwise be interpreted as a hypoglycemic event when converted to glucose estimates. For example, where the samples are measurements of current from an analyte electrode 302 measuring glucose, the threshold may be less than 600 pico Amperes (pA), less than 500 pA, or less than 400 pA. For example, the threshold may be less than or equal to 325.52 pA.

The compensation logic 804 may adjust the glucose samples 702 and or lactate samples 704 when the classification 802 indicates a compensatable compression event. Compensation may include applying a function to both the glucose samples 702 and lactate samples 704 to obtain adjusted values for the glucose samples. For example, the function may increase a glucose sample in correspondence with a magnitude of a mean glucose value and the value of the glucose sample and a magnitude of the corresponding lactate sample (e.g., same sample period) and a mean lactate value. The mean glucose value and mean lactate value may be for a time window preceding the time window in which the compression event is detected. In other embodiments, the glucose sample may be adjusted by substituting the mean glucose value or obtaining a weighted average of the glucose sample and the mean glucose value. Adjusting the glucose sample may include processing the glucose samples using a smoothing filter, such as a Kalman filter.

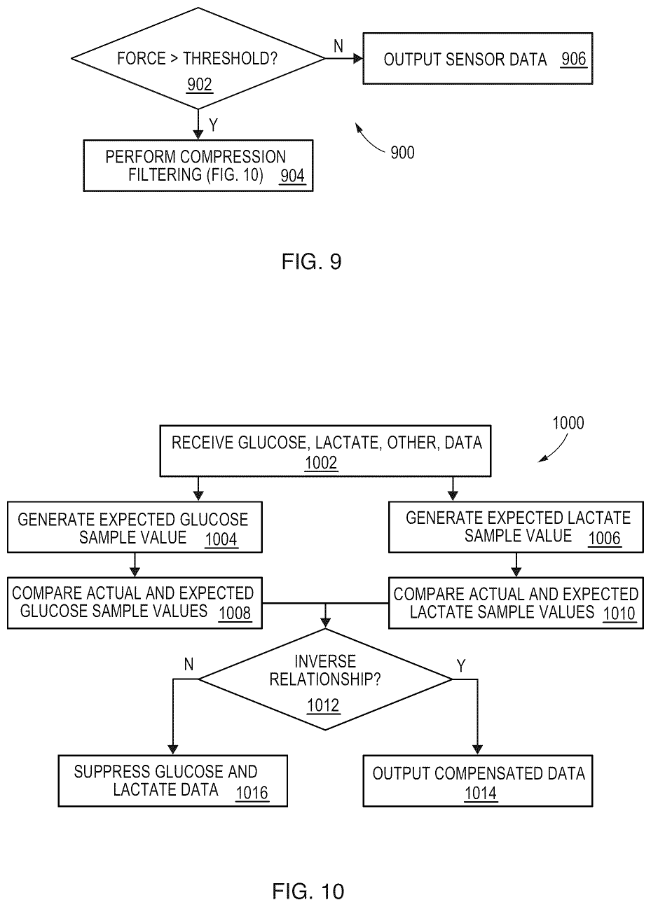

FIGS. 9 and 10 illustrate methods that may be used in place of or along with the compression classifier 800. Referring specifically to FIG. 9, in embodiments where the non-analyte sensors 206 include a force sensor, the method 900 may be used to make an initial assessment as to whether a compression event may have occurred. If the output of the force sensor is found, at step 902, to exceed a threshold, then compression filtering is performed at step 904. Compression filtering may include performing filtering such that glucose measurements that are artificially low due to compression events are either adjusted or blanked. An example approach for performing compression filtering is described below with respect to FIG. 10.

If the force is not found to be greater than the threshold, the method 900 may include outputting, at step 906, sensor data from the continuous analyte sensors 202 to the decision support engine 152 without performing compression filtering.

FIG. 10 illustrates a method 1000 for performing compression filtering. The method 1000 may be invoked according to the method 900 or based on some other criteria. For example, the method 1000 may be performed in all cases with the compression filtering being performed such that, in the absence of a compression event, the effect of the compression filtering will be insignificant or function only to smooth the samples output from the continuous analyte sensors. A criteria for invoking the method 1000 may include the current output of the electrode 302 detecting glucose falling below 300 pA, less than 200 pA, or less than 100 pA, such as below 90 pA for a single sample, all samples within a time window (e.g., 5, 10, or 60 minutes) or a threshold percentage of samples within the time window (e.g., at least 60 percent, at least 80 percent, or at least 90 percent).

The method 1000 includes receiving, at step 1002, a stream of data. The stream of data may include various types of data including glucose samples 702 and lactate samples 704. The glucose samples 702 and lactate samples 704 may be raw data (e.g., current measurements from analyte electrodes 302, 304) or processed data (e.g., estimates of glucose and lactose concentration). In some embodiments, other data is included in the stream of data such as some or all of force data 706, exercise data 708, drug data 710, and diet data 712.

Step 1002 may include receiving the samples for glucose and lactate for a current sample period (n), which are referred to herein as Ĝ[n] and \hat{L}[n], respectively. Other types of data such as force data 706 (F), exercise data 708 (E), diet data 712 (D), and drug data 710 (Dr) may also be time varying but at a slower rate such that such data does not for multiple sample periods. Accordingly, step 1002 may include receiving updated values for these types of data or using the same data from a previous iteration of the method 1000.

The method 1000 includes generating, at step 1004, an expected glucose sample value and generating, at step 1006, an expected lactate sample value. For example, steps 1004 and 1006 may include evaluating equations (1) and (2) to calculated predicted first derivatives for glucose and lactate (Ġ[n] and \dot{L}[n], respectively) for the current sample period (n). Expected values for the current samples of glucose and lactate (G[n] and L[n]) may then be calculated according to equations (3) and (4) based at least in part on previous values for glucose and lactate (Ĝ[n−1] and \hat{L} [n−1]). The values of Ġ[n−1] and \dot{L}[n−1] used in equations (3) and (4) may be values calculated according to (1) and (2) in a previous iteration or calculated based on actual values: Ġ[n−1]=(Ĝ[n−1]−Ĝ[n−2])/Δt and \dot{L}[n−1]=(\hat{L}[n−1]−L[n−2])/Δt, where Δt is the duration of the sampling period for the glucose and lactate samples, such as a value between 1 minute and 20 seconds, such as 30 seconds. The coefficients A, B, C, M, N, O, P, Q, R, S, T and U may be determined using previously received data for the user of the continuous analyte monitoring system 104 or for a different user or group of users. The coefficients may be calculated using a curve fitting algorithm, linear regression, or other technique.

$$\dot{G} [ n ] = A * F + B * \dot{L} [ n - 1 ] + C * \hat{G} [ n - 1 ] + M * E + N * D + 0 * Dr \qquad ( 1 )$$

$$\dot{L} [ n ] = P * F + Q * \dot{G} [ n - 1 ] + R * \hat{L} [ n - 1 ] + S * E + T * D + U * Dr \qquad ( 2 )$$

$$G [ n ] = \hat{G} [ n - 1 ] + \dot{G} [ n - 1 ] * Δ t \qquad ( 3 )$$

$$L [ n ] = \hat{L} [ n - 1 ] + \dot{L} [ n - 1 ] * Δ t \qquad ( 4 )$$

The method 1000 may include comparing the actual and expected sample values for glucose at step 1008 and comparing the actual and expected sample values for lactate at step 1010. In particular, steps 1008 and 1010 may include calculating difference values, such as ΔG=G[n]−Ĝ[n] and ΔL=L[n]−\hat{L}[n], respectively.

The method 1000 may include evaluating, at step 1012, whether the ΔG and ΔL exhibit an inverse relationship, i.e., opposite signs. Step 1012 may include evaluating whether ΔG and ΔL exhibit a specific inverse relationship: falling glucose (negative ΔG) and rising lactate (positive ΔL) corresponding to a compression event as shown in FIG. 5. Step 1012 may require that the inverse relationship or the specific inverse relationship be true for multiple sample periods n or for a percentage (e.g., at least 50, at least 70, or at least 90) of the sample periods within a time window, such as a 5, 10, 20, 30 or 60 minute window.

If the inverse relationship is found, the method 1000 may include outputting, at step 1014, compensated data. Compensated data may include outputting the expected values for glucose and/or lactate (G[n] and L[n]) rather than the actual sample values Ĝ[n] and \hat{L}[n]. Compensated may include outputting an average or weighted average of the expected values for glucose and/or lactate (G[n] and L[n]) and the actual sample values Ĝ[n] and \hat{L}[n].

The method 1000 is an example implementation of a compression filter. Other approaches may also be used. For example, a Kalman filter may be used to calculate expected values and also be used to compensate for compressions using the inherent smoothing provided by a Kalman filter. Other filtering or smoothing approaches may also be used to calculate the expected values for glucose and lactate.

If the inverse relationship is not found at step 1012, the method 1000 may include suppressing, at step 1016, output of the actual glucose and possibly lactate samples. In particular, where an above-threshold force is detected according to the method 900 or based on some other criteria and the inverse relationship is not found, an acute compression event may have occurred (see FIG. 6) such that compensation is not helpful or possible. As noted above, suppressing may include outputting nothing or blanking a value for a sample period to indicate that the sample for the sample period has been suppressed.

In some embodiments, the method 1000 may be modified to omit the use of lactate sample values. For example, terms relating to lactate may be removed from equation (1). Step 1012 may be modified to omit evaluation of an inverse relationship between glucose and lactate. For example, step 1012 may be modified to include evaluating a magnitude of the difference ΔG between the actual and expected glucose sample values. For example, if ΔG is less than a difference threshold, step 1016 will be executed, otherwise step 1014 may be performed.

In another embodiment, a rate of change of the actual glucose sample value Ĝ[n] may be evaluated with respect to a rate of change threshold. For example, if a negative rate of change having a magnitude greater than the rate of change threshold, step 1016 will be performed, otherwise step 1014 may be performed.

In yet another embodiment, a difference between the actual glucose sample Ĝ[n] may be compared to a reference value, such as a moving average of actual glucose samples for a preceding time window, such as the last 5 minutes, 10 minutes, 20 minutes, one hour, or other time interval. If the actual glucose sample value Ĝ[n] is below the reference value by more than a reference threshold, step 1016 will be performed, otherwise step 1014 may be performed.

### On-Skin Devices

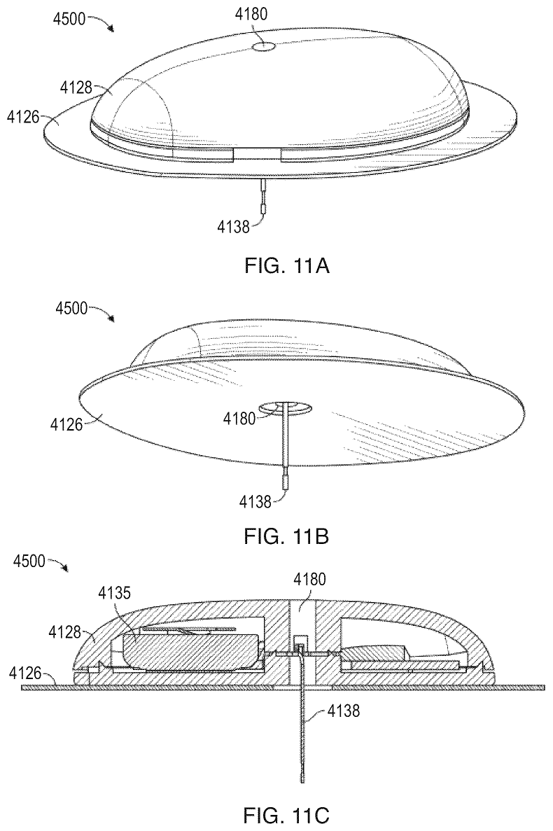

FIGS. 11A to 17 illustrate example on-skin devices. The features of the embodiments of FIGS. 11A to 17 are interchangeable with one another and may be used with any of the other embodiments disclosed herein.

FIGS. 11A, 11B, and 11C illustrate an exemplary implementation of analyte sensor system or continuous monitoring sensor system 104 implemented as a wearable device such as an on-skin wearable medical device or sensor assembly 4500. As shown in FIG. 11A, on-skin sensor assembly comprises a body in the form of a housing 4128. A patch 4126 can couple the housing 4128 to the skin of the host. The adhesive of the patch 4126 can be a pressure sensitive adhesive (e.g. acrylic, rubber based, or other suitable type) bonded to a carrier substrate (e.g., spun lace polyester, polyurethane film, or other suitable type) for skin attachment. The housing 4128 may include a through-hole 4180 that cooperates with an applicator such as a sensor inserter device (e.g., a sensor insertion needle, not shown) that is used for implanting sensor 4138 under the skin of a host.

The wearable sensor assembly 4500 can include electrical components in the form of sensor electronics (e.g., as at least a portion of electronics module 4135) operable to measure and/or analyze glucose indicators sensed by glucose sensor 4138. Sensor electronics within sensor assembly 4500 can transmit information (e.g., measurements, analyte data, and glucose data) to a remotely located device (e.g., 114, 116, 118, 120 shown in FIG. 1). As shown in FIG. 11C, in this implementation sensor 4138 extends from its distal end up into through-hole 4180 and is routed to electrical components in the form of an electronics module 4135 inside the enclosure or housing 4128. The working electrode 302 and/or 304 and reference electrode 306 are connected to circuitry in the electronics module 4135 which includes the potentiostat.

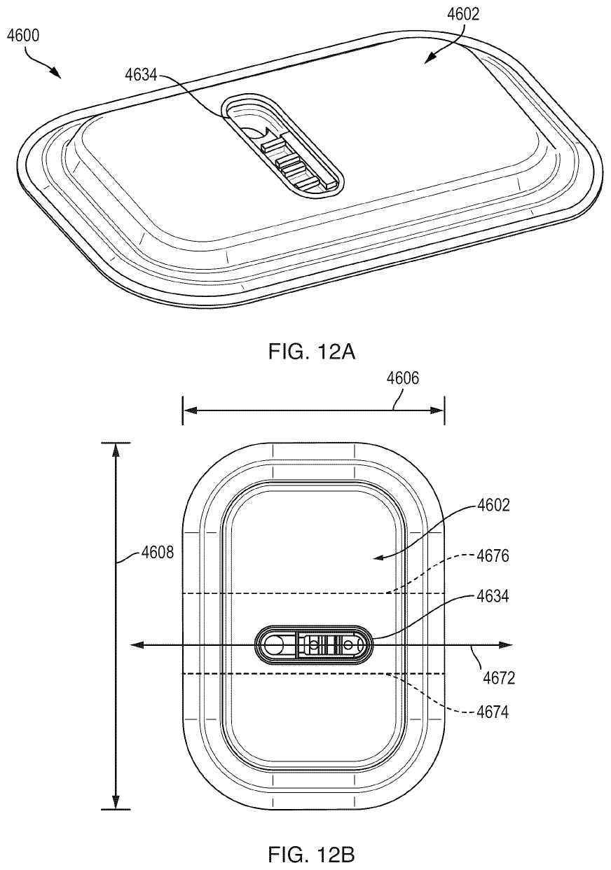

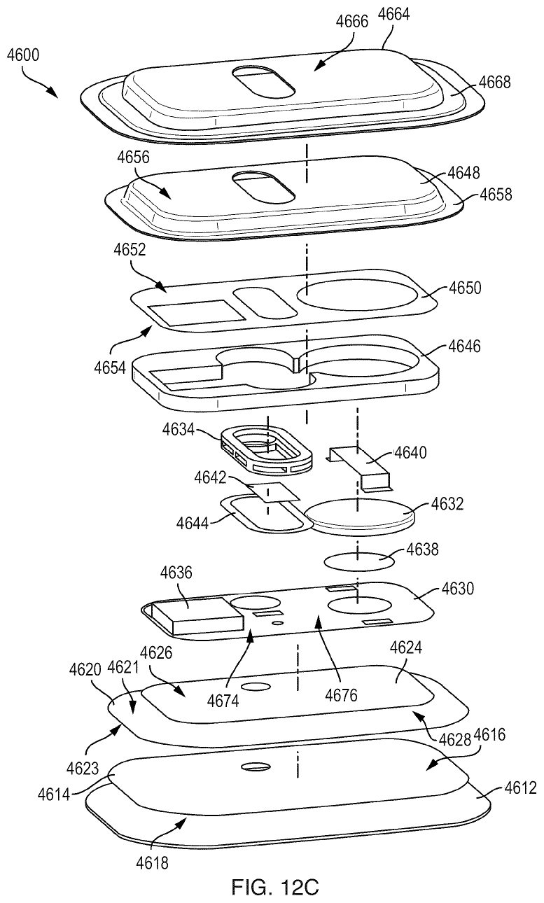

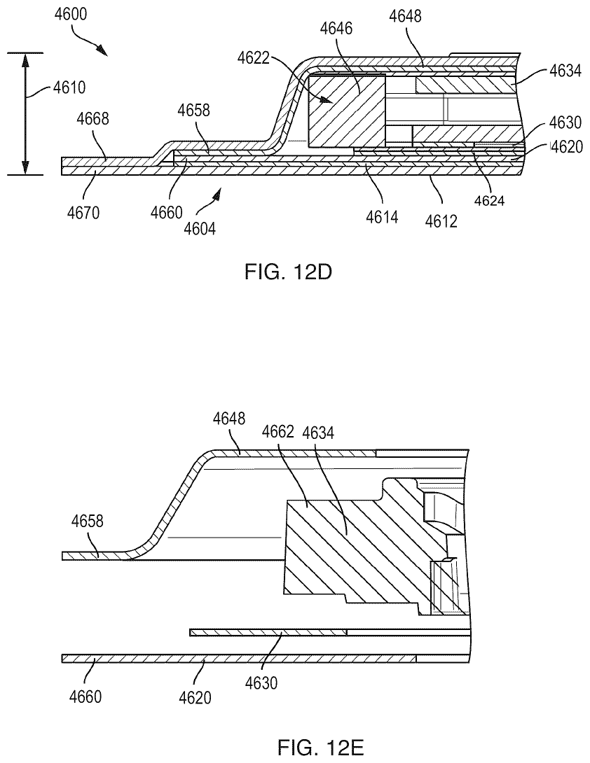

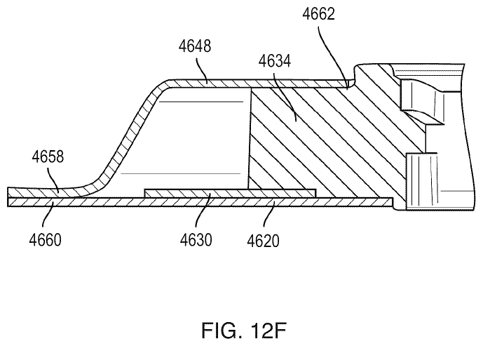

Variations in a configuration of a housing for the on-skin wearable medical device or sensor assembly may be provided. FIGS. 12A-12F, for example, illustrates an implantation including a body in the form of a housing 4600 configured to be worn on the skin and configured to couple to an analyte sensor or transcutaneous analyte sensor, the housing 4600 including a wall comprising a film layer. The body may be configured to be placed adjacent the skin of a host.

FIG. 12A illustrates a perspective view of the housing 4600, showing a top portion 4602 raised above a bottom portion 4604 (marked in FIG. 12D). The bottom portion 4604 may be configured to be proximate the skin of the host and the top portion may be raised from the bottom portion 4604 in a direction away from the skin of the host.

The housing 4600 may have dimensions including a width 4606, and a length 4608 (marked in FIG. 12B) and a height 4610 (marked in FIG. 12D). The length 4608 may be greater than the width 4606. The housing 4600 may have a rectangular shape as shown in FIG. 12B, or may have another shape (e.g., circular, triangular, hexagonal, or others) as desired. In implementations, the width 4606 and the length 4608 may each be greater than the height 4610. The housing 4600 may have a thin plate appearance with the height less than the width 4606 and length 4608. In implementations, the width 4606 or the length 4608 may each be less than the height 4610.

In implementations, the housing 4600 may have a construction including multiple components, or layers of components forming the housing 4600 and the on-skin wearable medical device or sensor assembly. The housing 4600 or the on-skin wearable medical device or sensor assembly may comprise an assembly of components. FIG. 12C, for example, illustrates an exploded perspective view showing components that may comprise the housing 4600 and the on-skin wearable medical device or sensor assembly. In implementations, features disclosed in regard to FIG. 12C may be excluded or substituted as desired.

A bottom portion of the housing 4600 may include a patch 4612 coupled to the housing 4600 and configured to couple the housing 4600 to skin of the host. The patch 4612, for example, may comprise a flexible material and may be configured to pass moisture therethrough. The patch 4612 may allow moisture to pass therethrough to enhance the breathability of the patch 4612. Such breathability may reduce adverse effects to the host's skin at the deployment site of the housing 4600 (e.g., ability to pass sweat or other moisture therethrough).

In implementations, the patch 4612 may have a same outer perimeter size as the housing 4600. As such, the patch 4612 may not protrude from the outer periphery of the housing 4600. FIG. 12D, for example, illustrates the patch 4612 extending to the outer periphery of the housing 4600. In implementations, the patch 4612 may protrude from the outer periphery of the housing 4600.

Referring to FIG. 12C, a first adhesive layer 4614 may be provided that may couple the patch 4612 to the housing 4600. The first adhesive layer 4614, for example, may comprise dual sided adhesive, with an adhesive on a top surface 4616 of the first adhesive layer 4614 and on a bottom surface 4618 of the first adhesive layer 4614. Other configurations of adhesive layers 4614 may be utilized as desired.

The first adhesive layer 4614 may couple the patch 4612 to a bottom film layer 4620 of the housing 4600. The bottom film layer 4620, for example, may comprise a wall of the housing 4600 that may seal an interior cavity 4622 (marked in FIG. 12D) of the housing 4600. The bottom film layer 4620 may include a top surface 4621 and a bottom surface 4623. In implementations, the bottom film layer 4620 may be configured to prevent moisture from passing therethrough, to reduce the possibility of moisture entering the interior cavity 4622 of the housing. The bottom surface 4623 may face towards the skin and at least a portion of the bottom surface 4623 may comprise a film layer.

A second adhesive layer 4624 may be positioned on the top surface 4621 of the bottom film layer 4620. The second adhesive layer 4624, similar to the first adhesive layer 4614, may comprise dual sided adhesive, with adhesive on the top surface 4626 of the second adhesive layer 4624 and adhesive on the bottom surface 4628 of the second adhesive layer 4624.

In implementations, the bottom film layer 4620 may comprise adhesive surfaces on one or more of the top surface 4621 or the bottom surface 4623 of the bottom film layer 4620 to couple the bottom film layer 4620 to the patch 4612 or the electrical substrate 4630. As such, one or more of the first adhesive layer 4614 or the second adhesive layer 4624 may be excluded as desired.

The housing 4600 may include one or more electrical components positioned therein. The electrical components may be positioned within an interior cavity of the housing. For example, an electrical substrate 4630 may be provided that may be configured to electrically couple to one or more other electrical components. In implementations, the electrical substrate 4630 may be configured to be flexible to allow for flexibility of the housing 4600. In implementations, the electrical substrate 4630 may include one or more bending sections that may be configured to allow the housing 4600 to bend at the bending sections. The housing 4600 may include one or more bending sections as may be disclosed herein. The housing 4600 may be flexible.

The electrical substrate 4630 may support electrical components thereon, including a power source such as a battery 4632, a socket 4634 for receiving a plug coupled to an analyte sensor or transcutaneous analyte sensor, and/or sensor electronics 4636 for processing a signal received from the analyte sensor or transcutaneous analyte sensor. The sensor electronics 4636, for example, may include a processor for processing signals from the sensor and may include a transmitter for transmitting signals to a receiver. The socket 4634 may include one or more electrical contacts or terminals for the sensor.

The power source such as the battery 4632 may be configured to power the electrical components within the interior cavity 4622 (marked in FIG. 12D). In implementations, a conductive tape 4638 may be configured to electrically couple the power source to the electrical components. As shown in FIG. 12C, the conductive tape 4638 may be positioned to contact a negative terminal of a battery. In implementations, conductive tape 4640 may be positioned to contact a positive terminal of a battery. The conductive tape 4640 may wrap around at least a portion of the battery 4632 to couple to the electrical substrate 4630 and provide power to the electrical substrate and the electrical components coupled to the electrical substrate.

The conductive tape 4640 may be flexible to enhance the flexibility of the housing 4600, and reduce the overall size and stiffness of the housing 4600. In implementations, the conductive tape 4640 may be omnidirectional and configured to allow current to flow therethrough in a variety of directions. Other forms of conductive tape may be utilized with the housing 4600.

Conductive tape 4642 may be utilized to electrically couple the socket 4634 to the electrical substrate 4630. The conductive tape 4642 may be uni-directional in embodiments, to allow electrical signals and power from the socket 4634 to flow in a desired direction between the electrical substrate 4630 and the socket 4634. In implementations, the conductive tape 4642 may include two sections, with a first section allowing for uni-directional flow to the socket 4634 from the electrical substrate 4630 and a second section allowing for uni-directional flow from the socket 4634 to the electrical substrate 4630. The first section and second section may be spaced from each other on a single piece of uni-directional conductive tape to avoid the possibility of electrical interference. In implementations, the conductive tape 4642 may include a cut or gap that physically isolates the first section and the second section to avoid electrical interference.

In implementations, other forms of electrical contacts between the socket 4634 and the electrical substrate 4630 may be utilized. For example, electrical contacts or terminals such as elastomeric pucks or other forms of contacts or terminals may be utilized to connect the socket 4634 and the electrical substrate 4630. The conductive tape 4642 accordingly may be excluded from use in embodiments, or utilized in combination with other forms of electrical contacts. Electrical contacts or terminals as disclosed in implementations herein may be utilized.

Referring to FIG. 12C, a third adhesive layer 4644 may be provided that may couple the socket 4634 to the electrical substrate 4630.

A filler 4646 may be positioned within the interior cavity 4622 (marked in FIG. 12D) of the housing 4600. The filler 4646 may comprise a fill layer that is configured to fit between the bottom film layer 4620 and the top film layer 4648 and may be shaped to fill voids that may surround the electrical components within the housing 4600. For example, the filler 4646 may be shaped to contour to the power source in the form of the battery 4632, and may contour to the socket 4634, and may contour to the sensor electronics 4636. The filler 4646 may include cut-outs contouring to such components, with the voids or rest of the interior cavity 4622 filled by the filler 4646. The filler 4646 may have a height that raises no greater than the height of the electrical components. For example, the height of the filler 4646 may be at or less than the height of the socket 4634.

In implementations, the filler 4646 may comprise a relatively lightweight material and may be configured to not allow for passage of air through the material of the filler 4646. For example, the filler 4646 may comprise a closed cell foam or other material that does not allow for passage of air. Such a feature may be beneficial if the housing 4600 experiences air pressure changes. A material such as closed cell foam may reduce the possibility of expansion of air within the housing 4600 according to a reduced air pressure exterior of the housing 4600. As such, the possibility of rupture or other damage to the housing 4600 may be reduced.

In implementations, the filler 4646 may comprise a compliant material that may provide impact protection for the housing 4600.

A fourth adhesive layer 4650 may be provided that may couple the filler 4646 to the top film layer 4648. The fourth adhesive layer 4650, similar to the first and second adhesive layers, may comprise dual sided adhesive, with adhesive on the top surface 4652 of the fourth adhesive layer 4650 and adhesive on the bottom surface 4654 of the fourth adhesive layer 4650. In implementations, the top film layer 4648 may comprise a top or bottom adhesive surface, and thus the fourth adhesive layer 4650 may be excluded as desired.

The top film layer 4648 may comprise a top portion of the housing 4600 and may have a similar construction as the bottom film layer 4620. The top film layer 4648 may have a contoured shape with a raised portion 4656 that is raised relative to a flange 4658 that extends about the outer periphery of the top film layer 4648. The flange 4658 may couple to the bottom film layer 4620 to seal the interior cavity 4622 marked in FIG. 12D.

For example, referring to FIG. 12D, the flange 4658 may couple to an outer portion 4660 of the bottom film layer 4620. The coupling of the top film layer 4648 to the bottom film layer 4620 may extend around the entirety of the interior cavity 4622 and about the entire periphery of the housing 4600. The coupling may occur in a variety of manners, including thermal welding, ultrasonic welding, or other forms of welding or coupling. The seal of the interior cavity 4622 may be moisture impermeable to reduce the possibility of moisture entering the interior cavity 4622.

Referring to FIGS. 12E and 12F, in implementations, the top film layer 4648 and the bottom film layer 4620 may each couple to the socket 4634. For example, the socket 4634 may include a peripheral portion 4662 that may extend around the central cavity of the socket 4634. The peripheral portion 4662 may comprise a flattened portion for the top film layer 4648 and the bottom film layer 4620 to each abut to seal against the socket 4634. As such, moisture impermeability may be provided around the socket 4634.

Referring back to FIG. 12C, a cover layer 4664 may be provided and may comprise an outer top surface of the housing 4600. The cover layer 4664 may be positioned over the top film layer 4648. The outer top surface may be configured to face away from the skin. Δt least a portion of the outer top surface may comprise a film layer. In implementations, the cover layer 4664 may comprise a film layer of the housing 4600. In implementations, the cover layer 4664 may have other forms. The cover layer 4664, similar to the top film layer 4648, may include a raised portion 4666 that is raised above a flange 4668 extending about the periphery of the cover layer 4664. The flange 4668 may be configured to couple to an outer portion 4670 of the patch 4612, as shown in FIG. 12D for example.

In implementations, the cover layer 4664 may comprise a smooth layer providing a smooth, low friction, outer surface for the housing 4600. The cover layer 4664 may further provide improved aesthetics for the housing 4600.

As discussed, at least a portion of the top portion 4602 of the housing 4600 may include the film layer, or at least a portion of the bottom portion 4604 (marked in FIG. 12D) of the housing 4600 may include the film layer. Both the top portion 4602 and bottom portion 4604 may include the film layer in implementations, and the film layers may be coupled to each other to form a seal of the interior cavity 4622.

Each film layer may be flexible in implementations, and as such, the housing 4600 may be flexible in implementations. The flexibility of the housing 4600 may allow for improved contour to the skin of a host, and for flexibility upon movement of the skin. The housing 4600 may have a relatively thin profile, allowing for flexibility in directions both towards and away from the skin.

Referring to FIGS. 12A and 12B, in implementations, the socket 4634 may comprise an opening on the top portion 4602 of the housing 4600. The socket 4634 may be exposed and comprise an opening on the top outer surface of the housing 4600. The socket 4634 may be configured to receive a plug coupled to an analyte sensor or transcutaneous analyte sensor. The plug may be configured to be inserted into the socket 4634 in a direction from above the housing 4600 downward towards the socket 4634. In implementations, the socket 4634 may have an oblong shape, with a long dimension 4672 of the socket 4634 extending along the width 4606 of the housing 4600. Such an orientation may provide a variety of benefits.

For example, the long dimension 4672 of the socket 4634 extending along the width 4606 of the housing 4600 may allow the electronic components (shown in FIG. 12C) to be positioned in areas separate from each other, with the flexible electrical substrate 4630 positioned therebetween. The electronic components may be spaced along the length 4608 of the housing 4600. For example, the sensor electronics 4636 may be spaced from the socket 4634, which may be spaced from the battery 4632. The orientation of the socket 4634 along the width 4606 of the housing 4600 accordingly may allow the housing 4600 to bend about the bending sections 4674, 4676 (marked in FIGS. 12B and 12C) positioned between these respective electronic components. The axes that the housing 4600 may bend about may extend parallel with the long dimension 4672 of the socket 4634 and along the width 4606 of the housing 4600.

In construction, the use of film layers may allow for high-speed, reel-to-reel manufacturing processes. For example, each of the film layers may comprise a flattened film sheet, which may be thermoformed or otherwise formed into a desired shape. The film layers may roll together and weld together in a manner that forms the housing 4600 (e.g., with the top film layer 4648 and the bottom film layer 4620 being drawn from a reel and being contacted with each other and welded together in a high-speed process). The other layers or components may be die cut and inserted into the layers during assembly. Such a configuration may allow for mass production of the housings 4600 and the components contained therein. Other manufacturing processes may be utilized as desired.

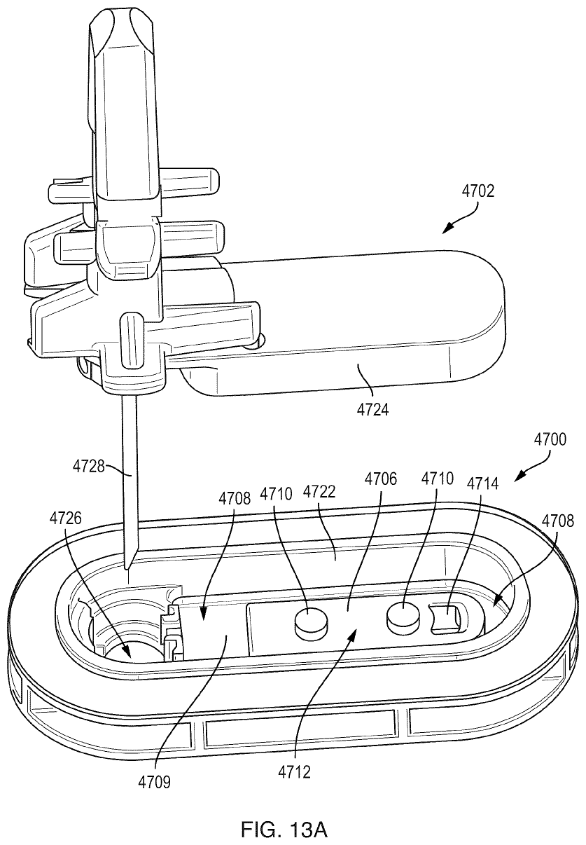

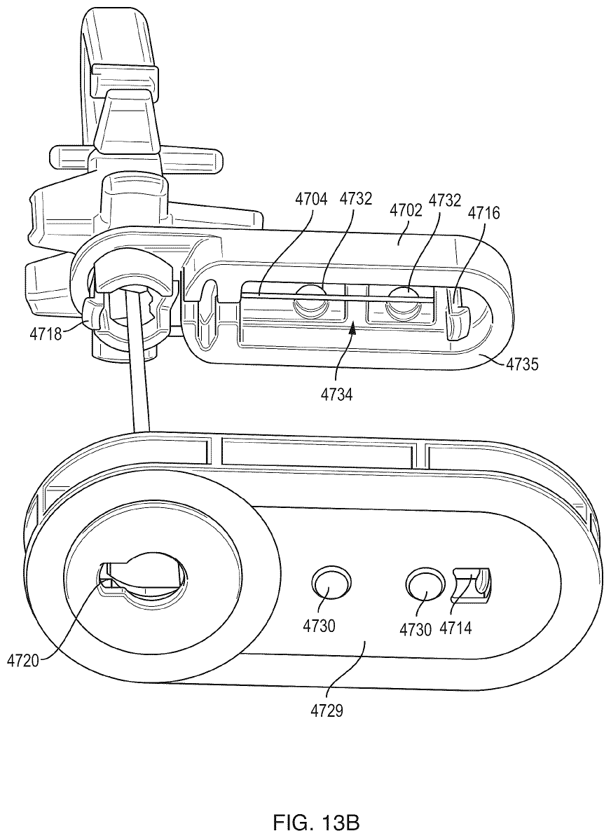

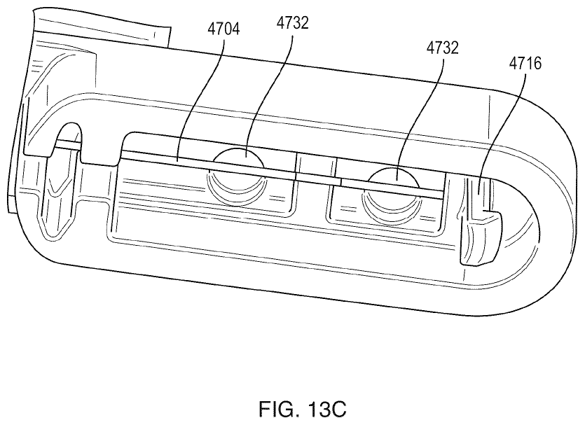

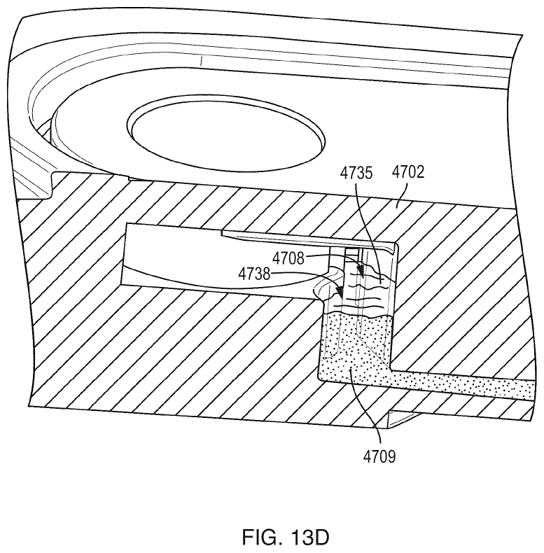

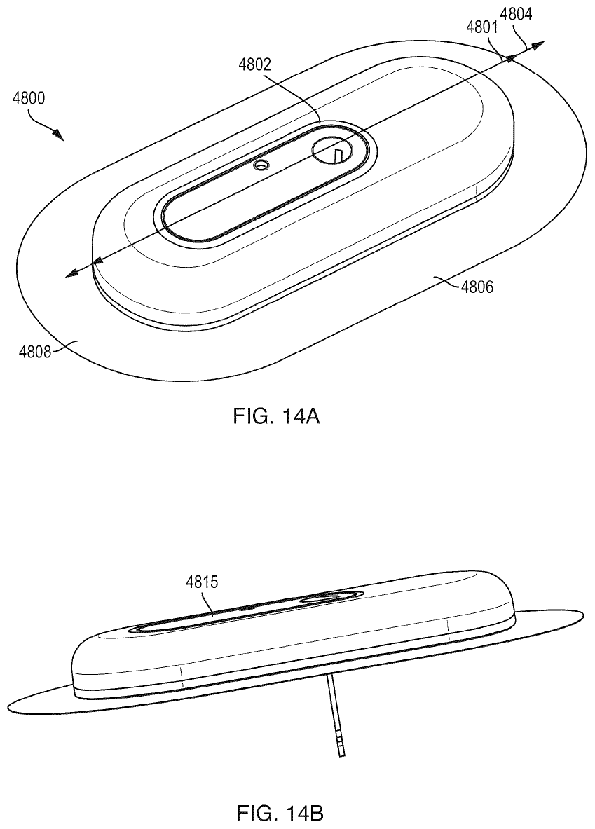

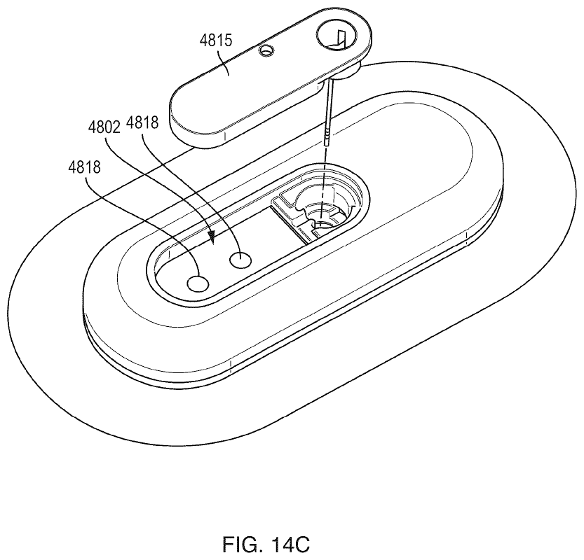

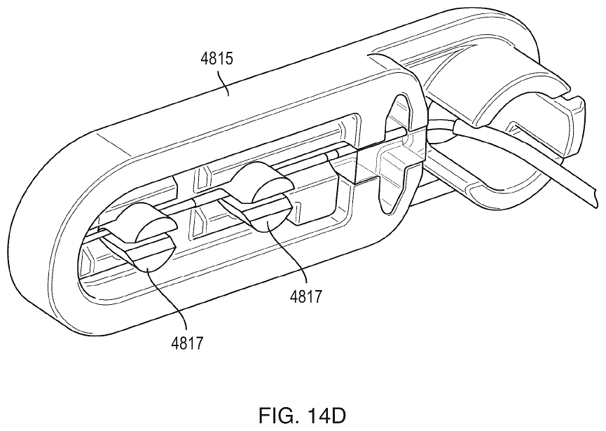

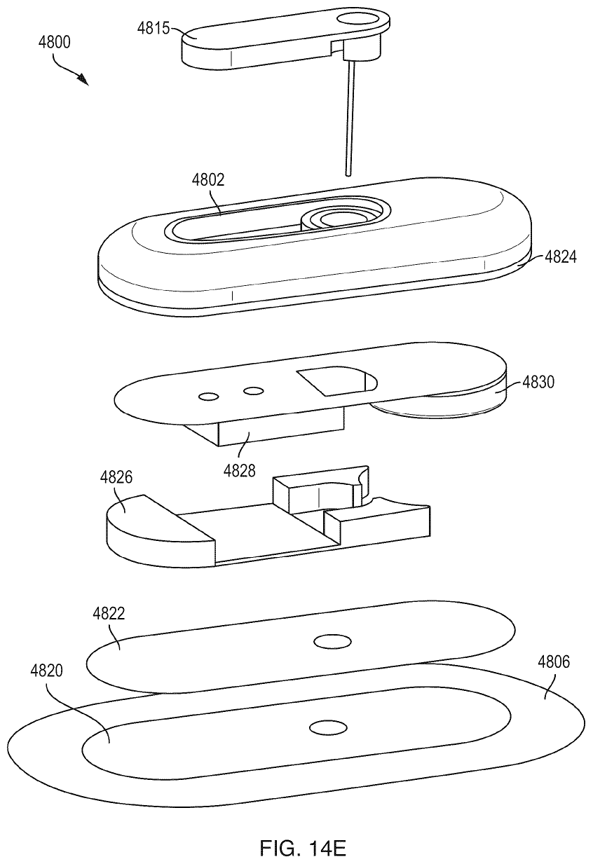

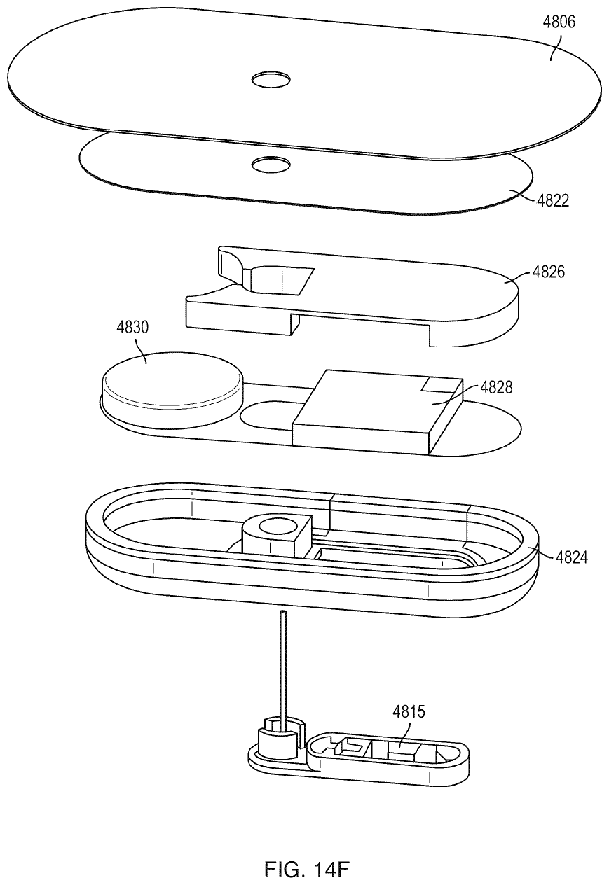

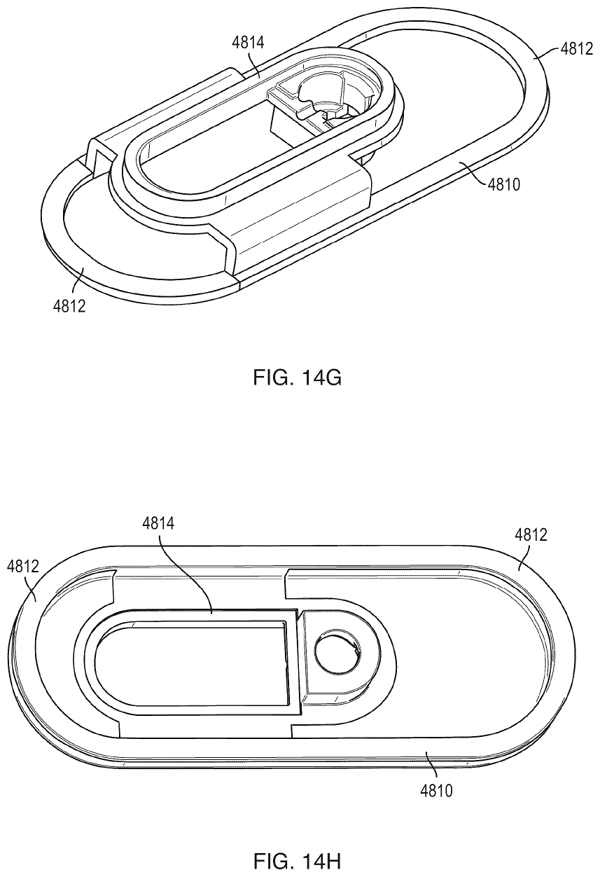

FIGS. 13A-13D illustrate an embodiment of a socket 4700 and a plug 4702 that may be utilized solely or in combination with implementations herein. The socket 4700, for example, may be utilized in implementations having a socket, such as the implementations of FIGS. 12A-12F, or the implementations of FIGS. 14A-14I.

Referring to FIG. 13A, the socket 4700 may be configured to be coupled to a body that may be worn on the skin, such as implementations of housings disclosed herein or another form of body. The socket 4700 may be configured to couple to the plug 4702 that may be coupled to an analyte sensor 4704 or transcutaneous analyte sensor as marked in FIG. 13B for example.

The socket 4700 may include a raised portion 4706 and a channel 4708 surrounding the raised portion 4706. The raised portion 4706 may include one or more electrical contacts 4710 for electrical connection with the analyte sensor 4704 and the channel 4708 may include a fluid 4709 disposed therein for forming a seal with at least a portion of the plug 4702.

The raised portion 4706 may be raised above the channel 4708 and may include a top surface 4712 that may have a flattened shape. The flattened shape of the top surface 4712, for example, may allow the electrical contacts 4710 to protrude from the top surface 4712 for connection with the analyte sensor 4704. The electrical contacts 4710 may be positioned on the top surface 4712 that is configured to face the plug.

The raised portion 4706 may include a first coupler 4714 in the form of an aperture for receiving a corresponding second coupler 4716 of the plug 4702 (marked in FIG. 13B). The first coupler 4714 may receive the corresponding second coupler 4716 to mechanically couple the plug 4702 to the socket 4700. The plug 4702 may include an additional third coupler 4718 (marked in FIG. 13B) that may couple to a fourth coupler 4720 in the form of an aperture for receiving the third coupler 4718. The second coupler 4716 and third coupler 4718 may comprise protrusions for engaging the apertures of the first and fourth couplers 4714, 4720, although other configurations may be utilized in embodiments. For example, one or more of the second coupler 4716 and third coupler 4718 may comprise apertures and one or more of the first and fourth couplers 4714, 4720 may comprise protrusions, although other configurations of couplers may be utilized as desired.

Referring back to FIG. 13A, the channel 4708 may comprise a lowered portion of the socket 4700 and may be bound by a raised outer wall 4722 surrounding the channel 4708. The raised outer wall 4722 may be configured to retain the fluid 4709 within the channel 4708. The raised outer wall 4722, in implementations, may have a greater height than the raised portion 4706, which may allow for alignment between the raised outer wall 4722 and the plug 4702. For example, the raised outer wall 4722 may contour to the shape of the peripheral surface 4724 of the plug 4702 to allow for alignment and sealing between the plug 4702 and the socket 4700.

The socket 4700 may further include an aperture 4726 for the analyte sensor 4704 to pass through, and for the insertion needle 4728 to pass through.

The fluid 4709, in implementations, may comprise a fluid for sealing the connection between the socket 4700 and the plug 4702 to reduce the possibility of moisture interfering with the electrical connection between the electrical contacts 4710 and the analyte sensor 4704. The fluid may be configured to be moisture impermeable, and as such may comprise a gel. The gel may comprise petroleum jelly or other forms of gel or fluids as desired. The fluid may be disposed within the channel 4708 and configured to be displaced upon the plug 4702 entering the socket 4700.

The electrical contacts 4710 may protrude from the top surface 4712 and in implementations may extend through the socket 4700 to pass to a bottom surface 4729 (marked in FIG. 13B) facing opposite the top surface 4712. The electrical contacts 4710 may include portions 4730 that protrude from the bottom surface 4729 and may be configured to electrically couple to electrical components, such as an electrical substrate or other electrical components. The electrical components, for example, may comprise a sensor electronics for receiving a signal from the analyte sensor 4704 or a power source. Other forms of electrical components may be utilized.

In implementations, the electrical contacts 4710 may comprise an electrically conductive elastomeric material. Such material may allow for compression of the electrical contacts 4710 upon contact and compression by the analyte sensor 4704. Other forms of electrical contacts may be utilized in implementations.

Referring to FIG. 13C, the plug 4702 may include recesses 4732 for electrically conductive portions of the analyte sensor 4704 to be positioned within and for the electrical contacts 4710 to enter. The recesses 4732, for example, may be shaped to receive the electrical contacts 4710 and allow for electrical connection between the contacts 4710 and the analyte sensor 4704. Portions of the analyte sensor 4704 that may be contacted may include a respective working electrode and reference electrode, although other portions may be contacted as desired.

The plug 4702 may include a cavity 4734 for the raised portion 4706 of the socket 4700 to enter into. The plug 4702 may include an outer wall 4735 for entering into the channel 4708 and surrounding the raised portion 4706.

In operation, the plug 4702 may be inserted into the socket 4700 to electrically connect the electrical contacts 4710 with the analyte sensor 4704. Upon insertion, the fluid 4709 may be displaced due to the presence of the outer wall 4735. FIG. 13D, for example, illustrates the outer wall 4735 positioned within the channel 4708.

In implementations, the socket 4700 may include a reservoir 4738 for receiving and storing any excess amount of the fluid 4709 following the coupling of the plug 4702 to the socket 4700. The reservoir 4738 may reduce the possibility of the fluid 4709 leaking upon insertion of the plug 4702 into the socket 4700. The configuration of plug 4702 and/or socket 4700 may be utilized with any implementation disclosed herein.

FIGS. 14A-14I illustrate an implantation of an on-skin wearable medical device or on-skin sensor assembly including an elongate housing 4800 having a long dimension 4801, and an elongate socket 4802 having a long dimension 4804 extending along the long dimension 4801 of the elongate housing 4800.

Referring to FIG. 14A, the elongate housing 4800 may be configured similarly as the housing 4600 shown in FIG. 12A unless stated otherwise. For example, the electrical components within the elongate housing 4800 may be comprise similar electrical components as the electrical components within the housing 4600.

The elongate housing 4800 may have an oblong shape and may be configured to be worn on the skin. For example, a patch 4806 may be utilized to couple the elongate housing 4800 to the skin. The patch 4806 may be coupled to a bottom portion of the elongate housing 4800 and may be configured to couple to elongate housing 4800 to skin. The patch 4806 may protrude outward from the outer periphery of the elongate housing 4800 to form a skirt portion 4808 extending radially outward from the elongate housing 4800.

The elongate housing 4800 may be constructed of one or more materials which may be rigid or may be flexible. In implementations, the elongate housing 4800 may be constructed of co-molded materials comprising a first material having a greater stiffness than a second material. For example, referring to FIG. 14G, a first portion of the elongate housing 4800 may comprise a frame 4810 constructed of the first material. The frame 4810 accordingly may comprise a relatively rigid body, including an outer peripheral loop 4812 and a central portion 4814 for supporting the socket 4802. FIG. 14H illustrates a bottom perspective view of the frame 4810.

The second material 4816 (marked in FIG. 14I) may be co-molded upon the frame 4810 and may comprise a flexible material. The second material 4816 may be coupled to the frame 4810. The second material 4816 may form at least a portion of an outer surface of the elongate housing 4800. The second material 4816 may improve the compliance of the elongate housing 4800 and may allow the elongate housing 4800 to be flexible, to contour to the shape of the host's skin and/or movement of the host's skin. Proportions of the elongate housing 4800 comprising the rigid first material and the compliant second material may be varied as desired to produce a desired flexibility for the elongate housing 4800. For example, in some embodiments, an outer housing is entirely constructed of a rigid material to form a rigid outer housing.

Referring to FIGS. 14B and 14C, the socket 4802 may be configured to receive a plug 4815 in a similar manner as other couplings of sockets and plugs disclosed herein. Referring to FIG. 14D, the plug 4815 may be configured similarly as the plug 702, yet may include protruding electrical contacts 4817 for coupling with the flattened electrical contacts 4818 of the socket 4802 shown in FIG. 14C.

The elongate housing 4800 may include a multi-level construction that may reduce the overall width and length of the elongate housing 4800 from a configuration as shown in FIGS. 12A-12F. However, the multi-level construction may increase the height of the elongate housing 4800 from a configuration as shown in FIGS. 12A-12F.

FIG. 14E, for example, illustrates a top perspective exploded view of the elongate housing 4800. The elongate housing 4800 may include the patch 4806, and an adhesive layer 4820 for coupling the patch 4806 to a bottom film layer 4822. The bottom portion of the elongate housing may include a film layer. The bottom film layer 4822 may be constructed similarly as the bottom film layer 4620 of FIGS. 12A-12F. The bottom film layer 4822 may couple to an outer periphery 4824 (marked in FIG. 14F) of the elongate housing 4800 to seal the interior cavity of the elongate housing 4800.

The elongate housing 4800 may include a filler 4826, which may be configured similarly as the filler 4646 of FIGS. 12A-12F. The multi-level construction of the elongate housing 4800 is shown with regard to the electrical components. The sensor electronics 4828 including the transmitter, for example, may be positioned directly below the socket 4802 and the plug 4815. The socket 4802 may be positioned above the transmitter. The sensor electronics 4828 may comprise a lower level of the elongate housing 4800 with the elongate socket 4802 comprising an upper level of the elongate housing 4800 positioned above the lower level. The elongate socket 4802 is accordingly positioned above the sensor electronics 4828 and transmitter in a multi-level configuration. In addition, the power source such as a battery 4830 may be positioned in the lower level with the sensor electronics 4828.

FIG. 14F illustrates an inverted view of the elongate housing 4800 from the configuration shown in FIG. 14E.

The multi-level construction may reduce the overall footprint, or length and width of the elongate housing relative to a configuration as shown in FIGS. 12A-12F for example. An increase in height, however, may result.

Referring to FIG. 14A, the extension of the long dimension 4804 of the elongate socket 4802 along the long dimension 4801 of the elongate housing 4800 may further reduce the overall footprint, or length and width of the elongate housing. This is in contrast to an implementation as shown in FIGS. 12A-12F, in which the long dimension 4672 of the socket 4634 extends perpendicular to the long dimension of the housing 4600. As such, reduced footprint of the elongate housing 4800 may result. The configuration of the elongate housing 4800 or elongate socket 4802 may be utilized with any implementation disclosed herein.

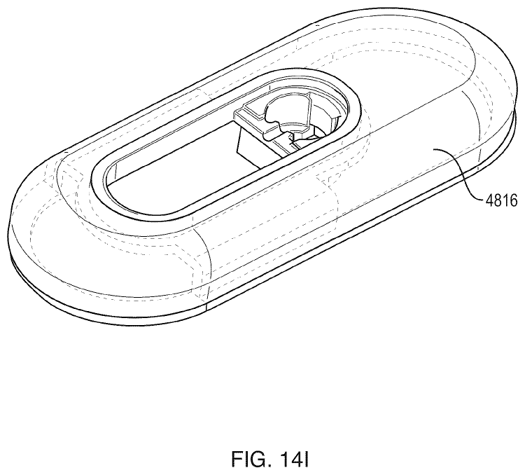

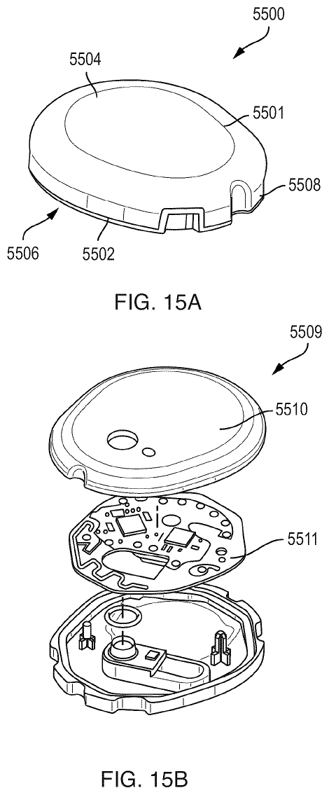

FIG. 15A illustrates a perspective view of an on-skin wearable medical device 5500 according to implementations herein. The device 5500 may comprise a body 5501 configured to be worn on the skin and configured to couple to a transcutaneous analyte sensor, in a manner as may be disclosed herein. The body 5501 may be configured as a housing. The body 5501 may be configured to retain one or more electrical components therein. The body 5501 may be configured to be placed adjacent to the skin of a host.

The body 5501 have a base 5502 and an enclosure 5504 coupled to the base 5502. The coupling may be facilitated via an adhesive in implementations, among other forms of coupling. In implementations, the coupling may create a seal between the base 5502 and the enclosure 5504. The seal may be air-tight and/or moisture-proof.

In some implementations, the adhesive may be a hotmelt adhesive or film including a thermoplastic (e.g., polyolefin). In some embodiments, the adhesive may be reactive polyurethane. A reactive polyurethane may be dispensed and moisture cured. In some embodiments, the adhesive may be silicone or epoxy. The silicone or epoxy may be cured using ultraviolet (UV) light to create a seal between the base 5502 and the enclosure 5504. Epoxy may be alternatively cured in an oven, which may be for about 30 minutes in an approximately 80 degrees Celsius temperature. Other forms of curing may be utilized. Similar to epoxy, acrylates may be used as an adhesive and cured under the same or similar conditions as epoxy using an oven. In some embodiments, a seal between the base 5502 and the enclosure 5504 may be achieved using welding. The welding techniques may include ultrasonic welding, laser welding, vibration welding, or electromagnetic welding.

At least a portion of the body 5501 may be made of a liquid crystal polymer (LCP). In implementations, for example, the enclosure 5504 may be entirely or partially made of a liquid crystal polymer (LCP). In other implementations, the enclosure 5504 may be completely or partially made of one or more of the following: polyproplene (PP), polyethylene terephthalate glycol (PETG), polycarbonate (PC), copolyester (CP), and cyclic olefin copolymer (COC). The enclosure 5504 may have low oxygen absorption and low moisture absorption. For example, the moisture absorption percentage may be between 0.01% and 0.06%. Preferably, the moisture absorption percentage may be 0.03%. Other amounts of absorption may be utilized. The enclosure 5504 may be biocompatible. The enclosure 5504 may be sterilizable. The enclosure 5504 may cover an entirety of the base 5502 such that the base 5502 is confined within the enclosure 5504.

The base 5502 may be entirely or partially made of PP, PETF, PC, CP, COC, or an LCP. The base 5502 may have a bottom surface 5506. The bottom surface 5506 may be facing away from the enclosure 5504. The enclosure 5504 may not extend over the bottom surface 5506. The bottom surface 5506 may be curved (e.g., concave) or flat. The bottom surface 5506 may be entirely or partially coupled to a patch (not shown). The patch may couple the body 5501 to the skin.

The base 5502 may retain one or more electrical components (for example as shown as electrical components 5511 in FIG. 15B) within a perimeter 5508 of the base 5502. The enclosure 5504 may extend over the electrical components. The one or more electrical components may include an electronics substrate (e.g., a printed circuit board, a flexible circuit board) and may include a power source, a transmitter, and/or a sensor electronically coupled to the electronics substrate. The electronic components may comprise sensor electronics. The power source (e.g., a battery, among other forms of power sources) may supply power to rest of the one or more electronic components.

The sensor may comprise an analyte sensor as disclosed herein, and may transcutaneously measure an analyte (e.g., glucose) in the blood of a user. The sensor may be configured to generate a signal indicative of an analyte concentration in a host. The sensor measurements may be communicated to the transmitter, which may then communicate the measurements to an output device. The body 5501 may retain one or more of the electrical components for receiving a signal from the analyte sensor. The output device may be a display, a computing device, or a portable electronic device by example. The user may view the measurements on a user interface of the output device and act based on the measurements (e.g., perform medical treatment, seek medical attention, consume food). The sensor may be configured to extend from the body 5501 to be positioned within the skin. The aforementioned features of the device 5500 discussed in this paragraph may be included in other on-skin wearable medical devices discussed throughout this disclosure.

FIG. 15B illustrates an exploded view of an on-skin wearable medical device 5509. The device may have the same specifications of the device of FIG. 15A, except the enclosure 5510 may be made of LCP entirely.

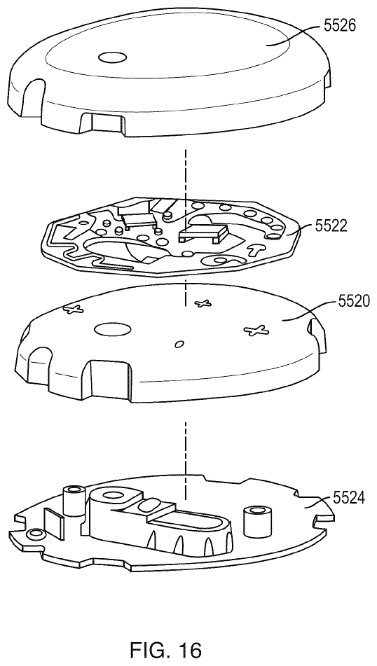

FIG. 16 illustrates an exploded view of an on-skin wearable medical device. The device may include one or more aspects of the device 5500 and may additionally include a filler 5520. In some implementations, the filler 5520 may be sandwiched between an electrical substrate 5522 and the base 5524. The electrical substrate 5522 in examples may include one or more electrical components such as a processor, a battery, and/or a memory storage. The filler 5520 may be sandwiched between the enclosure 5526 and the electrical substrate 5522. In some implementations, the filler may be positioned between a first housing and a second housing. The filler 5520, for example, may be disposed between the base 5524 and the enclosure 5526 or other forms of housings. The filler 5520 may occupy the empty space or voids between the enclosure 5526 and the base 5524. The filler 5520 may partially or completely surround electrical substrate 5522. The filler 5520 may comprise a thermoplastic and/or a thermoset polymer among other forms of material.

In some embodiments, filler 5520 may consist of, or include, a hotmelt material such as a thermoplastic polyamide or polyolefin. In such embodiments, the hotmelt material is configured to be molded at a low pressure, otherwise known as low pressure molding (LPM), which helps protect sensitive electronics and also cools or sets quickly to improve manufacturing throughput. The hotmelt material may cool or set more quickly than a thermoset polymer following molding. The hotmelt material may cool or set more quickly than the materials that the base and the enclosure are at least partially composed of following molding.

Enclosure 5526 and base 5524 may also be a molded component, but can be made using a different material or polymer using a different process. For example, enclosure 5526 and base 5524 can be molded from a thermoset polymer using conventional injection molding techniques, which occur at higher pressures and temperatures than an LPM process. In this manner, the polymers used to form enclosure 5526 and base 5524 can have different chemical and physical properties, such as improved hardness, improved cohesiveness, improved abrasion resistance, and/or reduced moisture permeability, when compared to a hotmelt material of an LPM process. The base 5524 and enclosure 5526 may be composed of a first material, and the hotmelt material may be configured to be molded at a lower pressure or temperature than the first material. The hotmelt material may comprise a filler composed of a different material than the base and/or enclosure. In some embodiments, enclosure 5526 and base 5524 can be molded from poly(methyl methacrylate) (PMMA), acrylonitrile butadiene styrene (ABS), nylon (polyamide, PA), polycarbonate (PC), polyethylene (PE), polyoxymethylene (POM), polypropylene (PP), polystyrene (PS), thermoplastic elastomer (TPE), thermoplastic polyurethane (TPU), and/or combinations thereof. The enclosure 5526 and base 5524 may be made of a different material or polymer than each other. In examples, the enclosure 5526 and base 5524 may be made of the same material.

In some embodiments, filler 5520 may consist of, or include, of a curable material. For example, the curable material can be a platinum-cured silicone, polyurethane, polysulfide, polyurea, epoxy, and combinations thereof. The curable material cures via an addition reaction, through the use of a catalyst, and/or a chemical reaction that does not require moisture.

In some embodiments, on-skin wearable medical device 5500 can be assembled or at least partially assembled according to the following method. In a first step, electrical substrate 5522 may be first placed either in enclosure 5526 or base 5524. Enclosure 5526 and/or base 5524 may include alignment and/or securement features configured to align and/or secure electrical substrate 5522. In a second step, the other of enclosure 5526 or base 5524 is assembled against the combined electrical substrate 5522 and enclosure 5526 or base 5524. In a third step, the assembled enclosure, 5526, base 5524, and electrical substrate 5522 is placed into a mold. In a fourth step, filler 5520 (e.g. hotmelt material) is injected into the cavity (e.g. via a LPM process) within the assembled enclosure 5526 and base 5524. In a fifth step, the filler 5520 is allowed to cool and set.

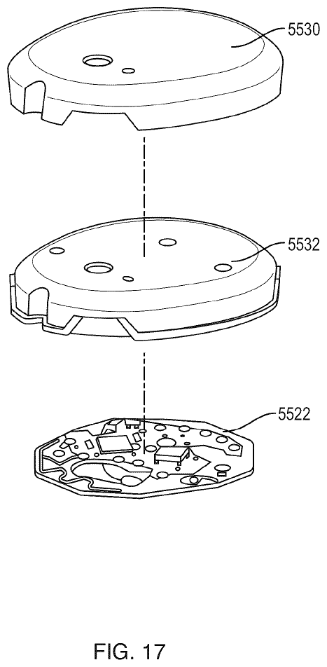

FIG. 17 illustrates an exploded view of an on-skin wearable medical device. The device may have an outer shell 5530, which may comprise a hardened outer shell 5530. The outer shell 5530 may comprise an outer shell 5530 of an enclosure and may extend over an inner shell 5532 of the enclosure. The outer shell 5530 may be made of LCP. Electronic components may be positioned within the housing (e.g., positioned upon the electrical substrate 5522). The inner shell 5532 may be made of one or more of a nylon plastic, a polyolefin, or a thermoplastic elastomer, among other materials. The outer shell 5530 may sit flush over the inner shell 5532. In implementations, the outer shell 5530 may be made of another material such as COC, or another material, by example.

Any implementation disclosed herein may include features of FIGS. 15A-17. Any implementation disclosed herein may utilize liquid crystal polymer (LCP) in a body, housing or other component as desired.

### Force Sensors

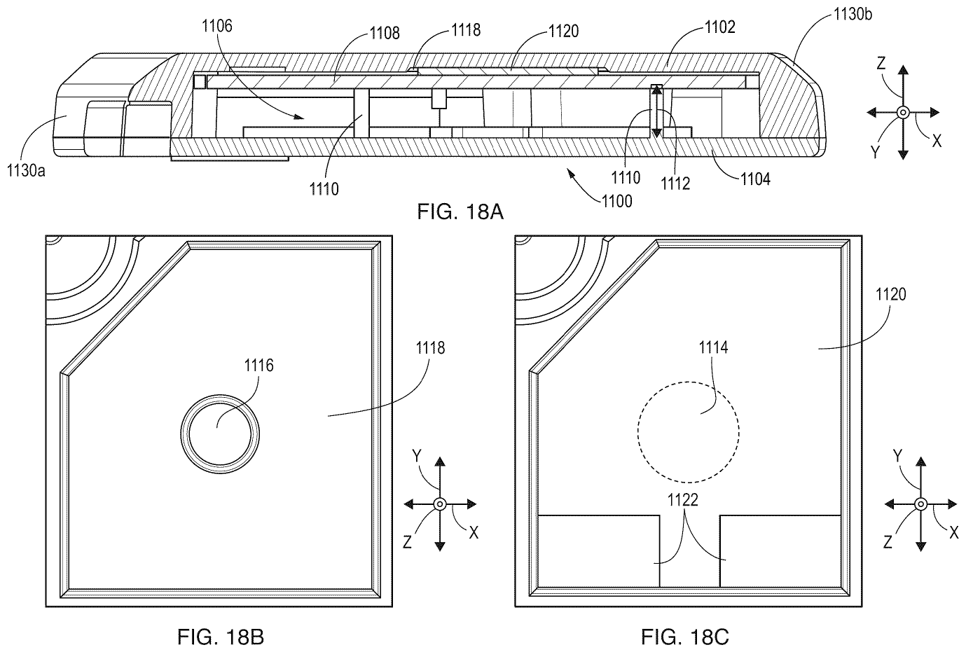

FIGS. 18A to 29F and 38 to 43 illustrate various approaches for detecting force exerted on the continuous analyte monitoring system 104. The detected force may be used as the force data 706 according to the methods described above.

A force-sensitive resistor (FSR) can have an active load range in which the resistance of the FSR changes in response to an applied load. The active load range can be defined by a lower threshold and an upper threshold. Δt loads below the lower threshold, the electrically active force-sensing portions of the FSR can be spaced apart from one another. Δt the lower threshold, the electrically active force-sensing portions of the FSR can be in contact with one another. At increasing loads above the lower threshold, the extent of contact between the electrically active force-sensing portions of the FSR also increases, such that increasing loads applied to the FSR above the lower threshold can be sensed with a proportional change in resistance of the FSR. As used herein, “proportional” may be understood as a decreasing monotonically with increase in the load, whether linearly, quadratically, or some other monotonic function. Likewise, “proportional” may be understood as having a slope within the active load range such that a change in loading of 5 N, 2 N, 1 N, or 0.5 N will produce a change in conductance (the inverse of resistance) of at least between 0.1 nS and 1 nS, between 0.1 μS and 1 μS, between 0.1 mS and 1 mS, between 0.1 S and 1 S, between 1 S and 10 S, between a range defined by any of these values, or within some higher or lower range of conductance.

At the upper threshold, the electrically active force-sensing portions of the FSR have been pushed together completely, such that additional loading on the FSR above the upper threshold has no further effect on resistance. In embodiments, to ensure an FSR can read small loads applied to an on-skin device, a continuous analyte monitoring system can be configured with a preload applied to the FSR that is at least as great as the lower threshold and substantially less than the upper threshold, such that externally-applied compressive forces on the system below the upper threshold can be measured.

FIGS. 18A to 18C illustrate an example implementation of a force sensor from which force data 706 may be received. FIG. 18A is a side cross sectional view of a continuous analyte sensor system including a force sensor. FIG. 18B is a partial bottom plan view of the upper housing of FIG. 18A, shown without the force sensor. FIG. 18C is partial bottom plan view of the upper housing of FIG. 18A, shown with the force sensor.

The continuous analyte sensor system 104 may include an on-skin medical device comprising a housing 1100 that may be understood with respect to an X, Y, and Z direction that are all perpendicular to one another. The Z direction may be defined as one or both of (a) substantially (e.g., within 5 degrees of) parallel to the component of compression that causes compression events and (b) normal to the skin of the user wearing the continuous analyte sensor system 104 at a center of the continuous analyte sensor system 104. The X and Y directions may be defined as substantially (e.g., within 5 degrees of) tangent to the skin to which the housing 1100 is mounted at the center of the continuous analyte sensor system 104.

The housing 1100 may include an upper housing 1102 and a lower housing 1104 defining a cavity 1106 therebetween. A circuit board 1108 can be mounted within the cavity 1106 and can support electronic components of the continuous analyte sensor system 104, such as one or more resistors, capacitors, inductors, integrated circuits, processing devices, a battery, or other components. The circuit board 1108, and other circuit boards of other embodiments disclosed herein, can likewise be coupled to a sensor incorporating electrodes of the continuous analyte sensor system 104 (see FIG. 3 and corresponding discussion). In some embodiments, electronic components of the continuous analyte sensor system 104 (see, e.g., FIG. 4 and corresponding description) can be mounted to a lower surface of the circuit board 1108, i.e., a surface facing toward the skin of a patient wearing the housing 1100.

The circuit board 1108 may be supported by one or more supports 1110. The supports 1110 may extend between the lower housing 1104 and the circuit board 1108. The circuit board 1108 may be secured to the supports 1110 or be secured due to being captured between the supports 1110 and the upper housing 1102. In some embodiments, the undeformed height 1112 of the supports 1110 in the Z direction is such that when the upper housing 1102 is secured to the lower housing 1104, one or more of the supports 1110 are compressed in the Z direction. For example, the undeformed height 1112 of the supports 1110 may be between 10 and 200 μm larger than the height of the supports 1110 when assembled in the housing 1100.

In some embodiments, as shown in FIGS. 18A-18C, an on-skin wearable medical device can include an FSR 1120. The device can be configured such that an external load applied to the upper housing 1102 in the Z direction will be at least partially transferred to an active area 1114 of the FSR 1120.

As shown in FIG. 18A, the FSR 1120 may be positioned between the circuit board 1108 and the upper housing 1102. The FSR 1120, or other force sensor according to any of the embodiments disclosed herein, may be coupled to electronic components within a housing (see, e.g., FIG. 4 and corresponding description), such as to the circuit board 1108 within the housing 1100.

The FSR 1120, or other force sensor according to any of the embodiments disclosed herein, may be calibrated following assembly of the continuous analyte sensor system 104 in order to increase the correspondence between a calibrated output of the force sensor and load applied to the continuous analyte sensor system 104.

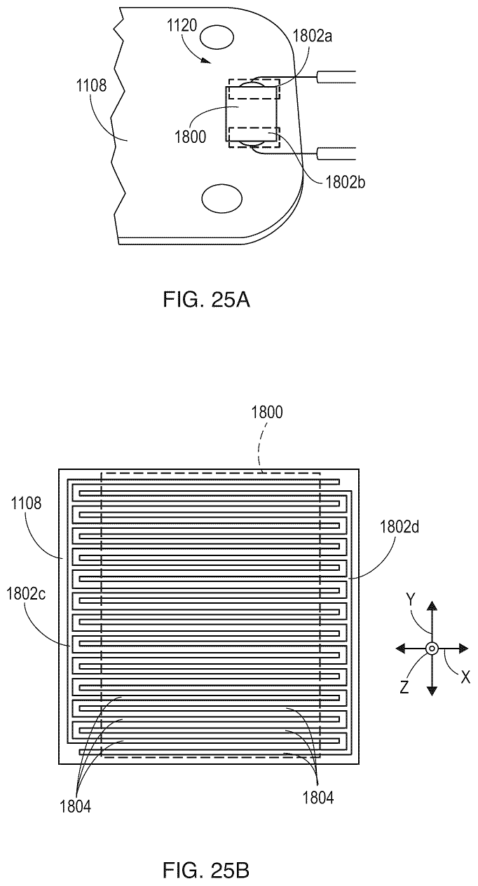

The FSR 1120 may be mounted within the housing 1100 such that forces in at least the Z direction will be sensed by the FSR 1120. The FSR 1120 may be implemented as a conductive polymer or other type of FSR known in the art. Other example implementations of an FSR are described below with respect to FIGS. 25A to 27B and FIGS. 38 to 43.

In the illustrated embodiment, the FSR 1120 is disposed within a recess 1118 of the upper housing 1102. A concentrator 1116 is positioned over the active area 1114 of the FSR 1120 and between the upper housing 1102 and the FSR 1120. In the illustrated embodiment, the concentrator 1116 comprises a protrusion extending from the upper housing 1102 and contacting the active area 1114 of the FSR 1120. The concentrator 1116 can have a cross-sectional area in the XY plane that is smaller than, or substantially the same size as, the active area 1114 of the FSR 1120. In some embodiments, the concentrator can have dimensions in the XY plane that are approximately (e.g., within 10 percent of) equal to the dimensions of the active area 1114 of the FSR 1120 in the XY plane.

In embodiments, the compression of the supports 1110 between the lower housing 1104 and the circuit board 1108 can cause the circuit board 1108 to press against the FSR 1120 from below, while the concentrator 1116 contacts the active area 1114 of the FSR from above, thereby resulting in a preload applied to the FSR 1120. As discussed above, preloading the FSR 1120 can bring the FSR close to or into its active load range, such that external compressive forces transferred to the FSR can be measured.

In some embodiments, the FSR 1120 is positioned in the XY plane offset from the supports 1110. In this manner, a load applied to the upper housing 1102 in the Z direction can tend to flex the circuit board 1108. In other embodiments, one or more supports 1110 can be aligned with the FSR (or aligned with the active area of the FSR) in the Z direction. In some embodiments, an FSR can be electrically coupled to and positioned over the circuit board 1108, with one or more support members disposed underneath the circuit board 1108, pressing the circuit board 1108 against the FSR from below. In other embodiments, an FSR can be electrically coupled to and positioned underneath the circuit board 1108, with one or more support members disposed underneath the FSR, pressing the FSR against the circuit board from below. In some embodiments, the supports 1110 can be also shaped and/or sized to deflect (e.g., compress or buckle) in a manner that transfers at least a portion of the load on the on-skin device to the FSR, resulting in a proportional change in resistance of the FSR 1120.

In embodiment illustrated in FIG. 18A, the circuit board 1108 is vertically offset from the upper housing 1102, such that the only portion of the upper housing 1102 in contact with the circuit board 1108 is the concentrator 1116. Also in the illustrated embodiment, the sidewalls 1130a, 1130b are monolithically formed with the upper housing 1102, but these may alternatively be monolithically formed with the lower housing 1104. The length of the concentrator in the Z direction may be between 10 microns and 1 mm, though smaller or larger sizes may also be used. In other embodiments, the circuit board can be in direct contact with the upper housing, and a concentrator can be omitted.

Referring again to FIG. 18A, the upper housing 1102 may define a recess 1118, with the concentrator 1116 extending into the recess 1118. The recess 1118 may have a size and/or shape configured to receive the FSR 1120. Although illustrated with recess 1118 having a roughly rectangular shape corresponding to the shape of the FSR 1120, embodiments can include a recess and/or FSR having any suitable shape, such as, for example, circular, oval, or other rectilinear or curvilinear shape, and the shape of the recess can be substantially the same as, or different from, the shape of the FSR. Further, in embodiments, one or more FSRs can be coupled to any other suitable structure within or on the outside of the housing of the on-skin medical device, with or without a recess in such structure.

The FSR 1120 may include electrodes 1122 coupled to an active area 1114 of the FSR 1120, through which resistance of the FSR 1120 is sensed or measured. The FSR 1120 can be hollow in the active area 1114. The electrodes 1122 may be connected to the active area 1114 through traces (not shown) in the FSR 1120. In embodiments, the FSR 1120 may be attached to the circuit board 1108 using any suitable technique. For example, the electrodes 1122 of the FSR 1120 may be connected to the circuit board 1108 using solder, a conductive pressure sensitive adhesive, conductive epoxy, or any other suitable approach.

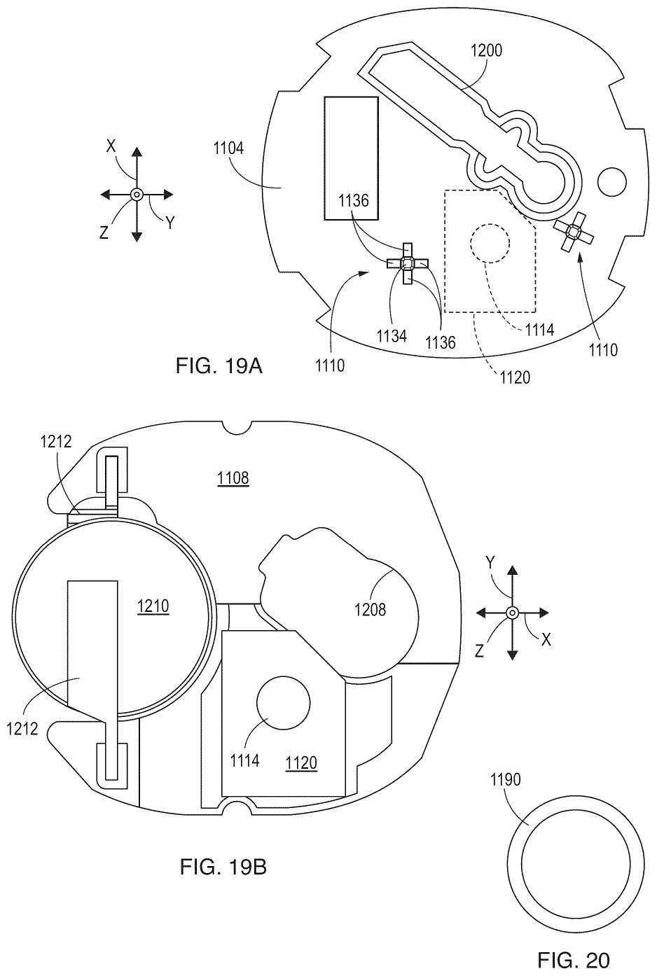

FIGS. 19A and 19B illustrate example layouts of components within the housing 1100. FIG. 19A is a top plan view of a lower housing of a continuous analyte sensor system, shown with a projection of a force sensor to illustrate an exemplary layout of components in the system. FIG. 19B is a bottom plan view of circuit board 1108 within a continuous analyte sensor system.

Referring specifically to FIG. 19A, the lower housing 1104 may define an opening 1200 for receiving a sensor (such as the plug 4815 of FIGS. 14C and 14D), which may be a preconnected sensor, and which may include a wire including electrodes as described above with respect to FIG. 3. The wire will be inserted into the skin of the patient during use. The positions in the XY plane of the supports 1110 and FSR 1120 may be offset from the opening 1200, such as by at least 1 mm, 2 mm, or 4 mm. In the illustrated embodiment, a line extending between the supports 1110 likewise does not extend across the opening 1200 in the XY plane. However, the FSR 1120 may advantageously be positioned proximate the opening 1200, specifically the portion of the opening 1200 through which the wire extends. Placing the force sensor in or near the center of the wearable can facilitate better transfer of the loads to the force sensor when a load is applied to the top of the wearable device. For example, the FSR 1120 may be positioned within 15, 10, 5, or 2 mm of the insertion site of the wire in the XY plane in use.

Referring specifically to FIG. 19B, the circuit board 1108 may likewise define an opening 1208 providing clearance for the sensor. The circuit board 1108 may define an opening or otherwise provide clearance for a battery 1210 and corresponding contacts 1212 for conducting current from the battery 1210 to the circuit board 1108.

The FSR 1120 may be arranged on the circuit board 1108 offset from the battery, the opening 1208 and possibly other components, such as one or more integrated circuits, as desired.

Referring again to FIG. 19A, the supports 1110 may have a variety of cross-sectional shapes, such as the illustrated supports 1110 including a central cylinder 1134 and one, two, three, or the illustrated four buttresses 1136 extending outwardly from the central cylinder 1134 and secured to the lower housing 1104. Such supports can be configured to undergo compression without buckling during a compression event, e.g., within the active load range of the FSR 1120. The buttresses 1136 can hinder buckling of the central cylinder 1134 such that any deformation of the supports 1110 will be due to compression rather than buckling. However, in certain other embodiments, the supports may be configured to buckle during a compression event. For example, buttresses 1136 may be omitted such that buckling of the support 1110 may occur when the device is compressed, e.g., within the active load range of loads of the FSR 1120.

FIGS. 20 to 23B illustrate other possible implementations for the supports. As shown in FIG. 20, which shows a top plan detail view of supports 1190 configured in accordance with some embodiments, supports 1190 comprise one or more hollow cylinders, for example having an outer diameter and/or wall thickness selected to provide a degree of compressive deflection and/or buckling to facilitate achieving the proportional change in resistance within the active load range of the FSR. To configure a support feature as a buckling feature, the preload on the support feature should exceed the critical buckling load of the support feature. To achieve a desired critical buckling load, a support can include an upper feature configured to transfer load eccentrically to the support. Eccentrically applied loads can effectively reduce the critical buckling load for a column, causing the support to preferentially buckle at a lower load. Another method of reducing the critical buckling load is to reduce the product of the elastic modulus and the moment of inertia of the column. An additional method to decrease the critical buckling load is to increase the length of the support. To configure the support in a way such that the support deflects in compression without buckling, the moment of inertia and elastic modulus can be selected to increase the critical buckling load, and/or the length of the support can be decreased.

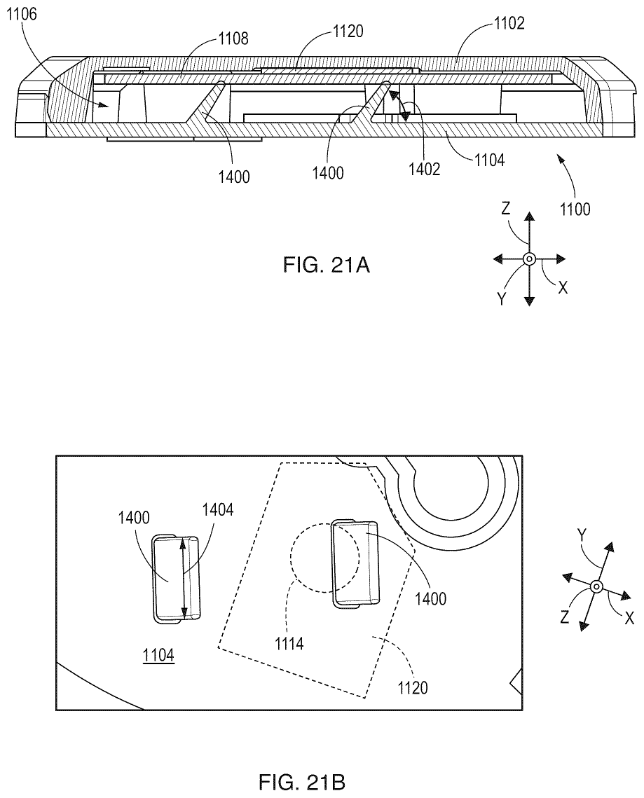

FIG. 21A is a side cross sectional view of a continuous analyte sensor system including a force sensor configured according to another embodiment. FIG. 21B is a partial top plan view of the lower housing of FIG. 21A, shown with a projection of the force sensor 1120 and its active area 1114 to illustrate another exemplary layout of components in the system. As shown in FIGS. 21A and 21B, embodiments may include supports 1400 comprising angled arms that are angled in a plane parallel to the Z direction, such as defining an angle 1402 with respect to the XY plane of between 20 and 50 degrees, or between 30 and 45 degrees. The supports 1400 may further taper in thickness with distance from the lower housing 1104. The supports 1400 may therefore comprise leaf springs to facilitate the proportional resistance within the range of loads. The undeflected height of the angled arms 1400 in the Z direction may be higher, such as by between 10 and 200 μm, than the lower surface of the circuit board 1108 that is contacted by the angled arms 1400 to provide a degree of preloading of the FSR 1120 in the absence of an external load on the upper housing 1102. As shown in FIG. 21B, the supports 1400 may have a width 1404 in the XY plane that may be equal or greater than the undeformed height of the supports 1400 in the Z direction. The supports 1400 may have a constant cross section along a major portion thereof, or may have any other suitable configuration.

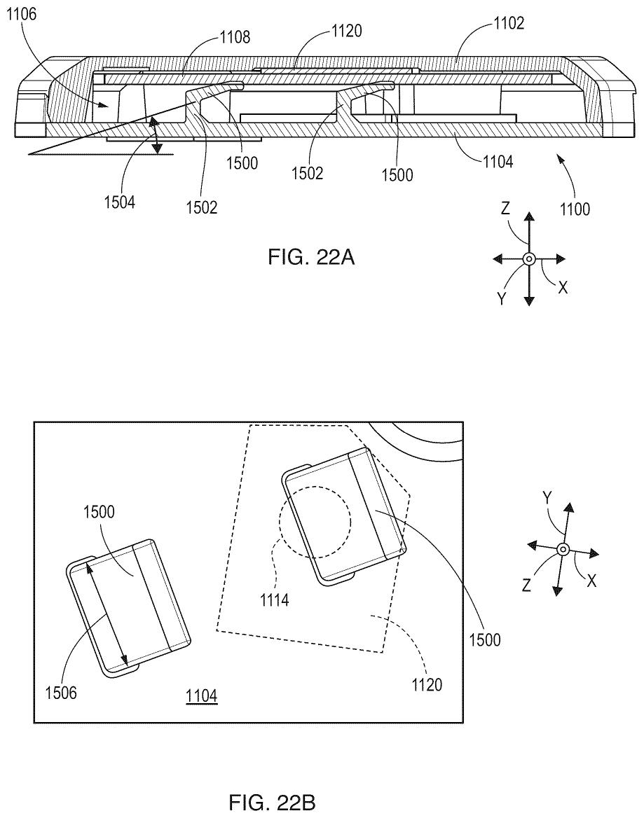

FIG. 22A is a side cross sectional view of a continuous analyte sensor system including a force sensor according to another embodiment. FIG. 22B is a partial top plan view of the lower housing of FIG. 22A, shown with a projection of the FSR 1120 and its active area 1114 to illustrate another exemplary layout of components in the system. As shown in FIGS. 22A and 22B, embodiments may include supports 1500 comprising arms extending from or otherwise secured to elevated bases 1502 such that angle 1504 defined by each arm relative to the XY plane is acute, such as between 0 and 45 degrees or between 10 and 30 degrees. The supports 1500 may therefore comprise leaf springs to facilitate the proportional resistance within the range of loads. The undeflected height of the supports 1500 in the Z direction may be higher, such as by between 10 and 200 μm, than the lower surface of the circuit board 1108 that is contacted by the supports 1500 to provide a degree of preloading of the FSR 1120 in the absence of an external load on the upper housing 1102. As shown in FIG. 22B the supports 1500 and elevated bases 1502 may have a width 1506 in the XY plane that may be equal or greater than the undeformed height of the supports 1500 in the Z direction. The arms 1500 and elevated bases 1502 may have a constant cross section along a major portion thereof, or may have any other suitable configuration.

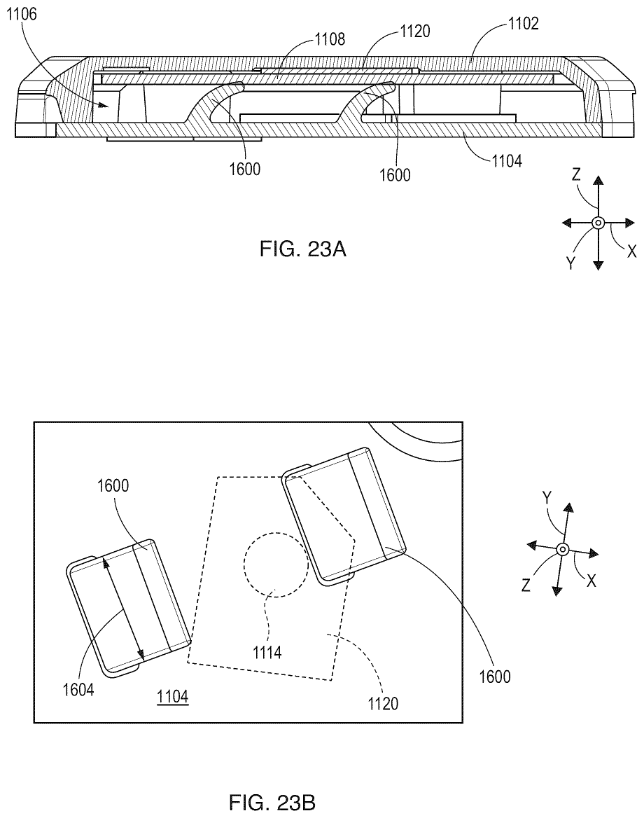

FIG. 23A is a side cross sectional view of a continuous analyte sensor system including a force sensor according to another embodiment. FIG. 23B is a partial top plan view of the lower housing of FIG. 23A, shown with a projection of the FSR 1120 and its active area 1114 to illustrate another exemplary layout of components in the system. As shown in FIGS. 23A and 23B, embodiments may include supports 1600 comprising arms that are curved in a plane parallel to the Z direction, such as having an inner radius of curvature equal to between 0.5 and 1.5 times a separation between the circuit board 1108 and the lower housing 1104 as measured in the Z direction. The supports 1600 may therefore comprise leaf springs to facilitate the proportional resistance within the range of loads. The undeflected height of the arms 1600 in the Z direction may be higher, such as by between 10 and 200 μm mm, than the lower surface of the circuit board 1108 that is contacted by the arms 1600 to provide a degree of preloading of the FSR 1120 in the absence of an external load on the upper housing 1102. As shown in FIG. 23B the supports 1600 may have a width 1604 in the XY plane that may be equal or greater than the undeformed height of the supports 1600 in the Z direction. The supports 1600 may have a constant cross section along a major portion thereof, or may have any other suitable configuration.

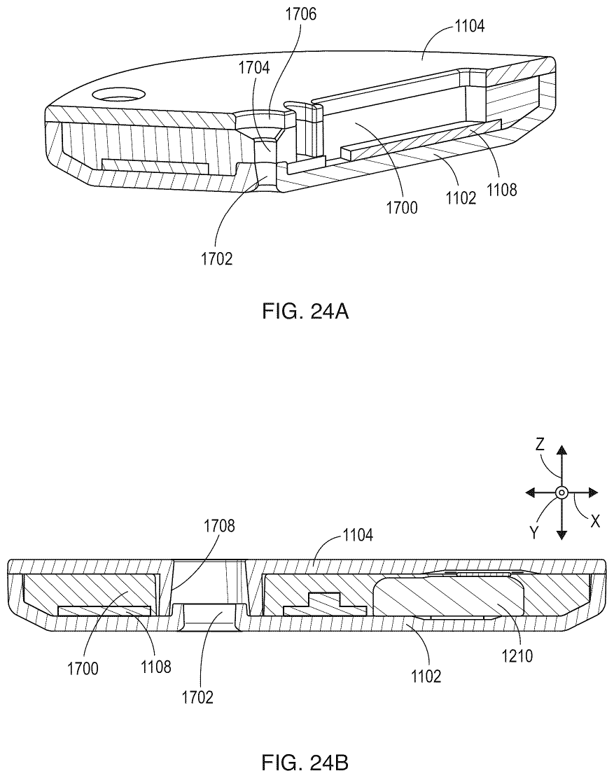

FIG. 24A is a partial cutaway cross-sectional view of a continuous analyte sensor system including a force sensor, configured in accordance with another embodiment, and shown with the skin-facing side of the lower housing 1104 facing upwards. FIG. 24B is a side cross-sectional view of a continuous analyte sensor system including a force sensor, configured in accordance with yet another embodiment, and shown with the skin-facing side of the lower housing 1104 facing upwards. As shown in FIGS. 24A and 24B, embodiments can include a compressible member 1700 disposed within the cavity 1106 between the circuit board 1108 and the lower housing 1104. The compressible member can be configured to transfer externally applied forces in the Z direction from the upper housing 1102 to the force sensor (not visible in FIGS. 24A and 24B). The compressible member 1700 can be used instead of, or in addition to, one or more supports and/or concentrators, which may be disposed on the same side or the opposing side of the force sensor, as described elsewhere herein.

In embodiments, the compressible member 1700 can comprise a structural foam, such as a closed-cell or open cell foam, or an elastomer, a polymer, or any combination thereof. Properties of the compressible member 1700 may be selected to achieve the desired proportional resistance within the desired active load range. Such properties may include the modulus of elasticity of the material used, the density, and/or features such as holes, columns, texturing, or other features that may collapse when the member is compressed. In some embodiments, the compressible member 1700 can have a hardness of between, for example, Shore 00-50 and 80 Shore D, 20 Shore A and 90 Shore A, or 30 Shore A and 80 Shore A, whereas the upper and lower housings 1102, 1104 can have a hardness of, for example, 55 Shore D or greater, or 80 Shore D or greater.

The compressible member 1700 may occupy all or substantially all (e.g., at least 90 percent of) of the volume of the cavity 1106 between the circuit board 1108 and the lower housing 1104. Alternatively, the compressible member 1700 may be disposed in or confined to an area between the circuit board 1108 and the lower housing 1104 including the extent of the circuit board 1120, such as extending no more than 1 to 5 mm from the extent of the FSR circuit board 1120, in the XY plane.

In this and other embodiments disclosed herein, structural foam, such as the structural foam 1700, may be replaced with a natural or synthetic material that is both pliable and provides a degree of structural strength, such as a natural or synthetic elastomer or polymer that likewise provides support and properties as described above for the structural foam according to any of the embodiments described herein.

FIGS. 24A and 24B further illustrate that an opening may extend through the housing 1100 for receiving a needle for implanting the sensor. In the embodiment illustrated in FIG. 24A, the opening is defined by an inner wall 1702 of the upper housing 1102, an inner wall 1704 of the compressible member 1700, and an inner wall 1706 of the lower housing. The stiffness of any or all of the inner wall 1702, inner wall 1704, and the inner wall 1706 can be selected to permit or resist flexing of the upper and/or lower housings 1102, 1104 and, accordingly, to promote or limit transfer of compressive loads through the upper and/or lower housings 1102, 1104 to the FSR. For example, the inner wall 1704 in FIG. 24A is formed by the compressible member 1700. In the embodiment illustrated in FIG. 24B, the opening through the housing is formed by an inner wall 1702 of the upper housing 1102 cooperating with an inner wall 1708 of the lower housing 1104.

Referring to FIGS. 25A and 25B, in some embodiments, an FSR 1120 according to any of the embodiments disclosed herein can comprise a conductive member 1800 disposed above or below two or more electrodes 1802.

In some embodiments, the conductive member 1800 can be compressible, and can comprise a conductive foam, a conductive elastomer, a conductive polymer, or any combination thereof. The conductive member 1800 can be configured such that the resistance of a conductive path between the electrodes 1802 decreases as the conductive member 1800 is compressed toward the electrodes 1802 due to one or both of (a) an increase in the area of the conductive member 1800 that is in contact with the electrodes 1802 and (b) increased density of conductive particles within the conductive member 1800. The bulk of the member 1800 in which the conductive particles are embedded may be an elastomeric foam or other type of foam. The conductive particles can comprise carbon (e.g., graphite) or other conductive material.

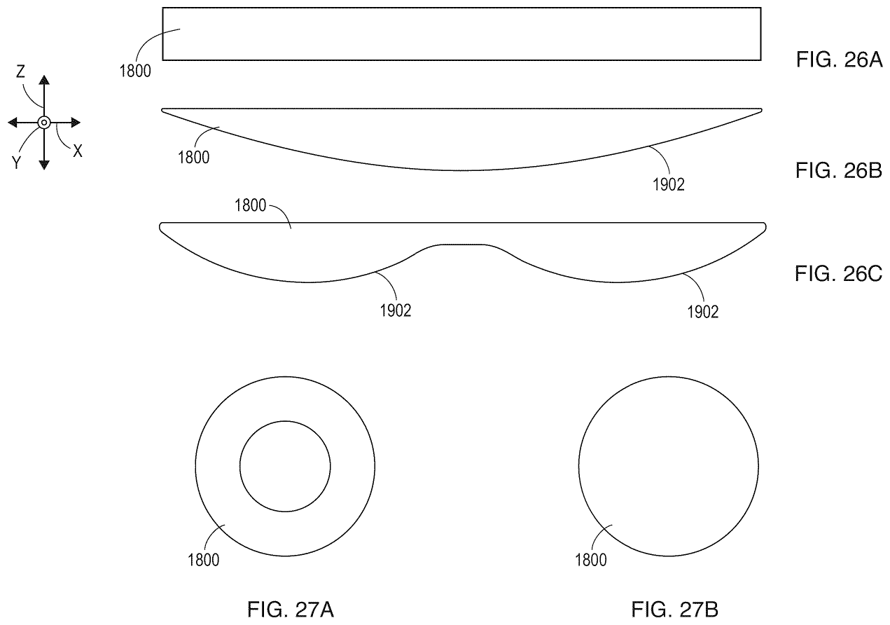

The change in resistance of the conductive member 1800 in response to compressive loading may be a function of the separation and/or configuration of the electrodes 1802 and properties of the conductive member 1800 itself, such as surface roughness, density of conductive particles, modulus of elasticity of the bulk material, density, and the cross-sectional shape of the foam in planes parallel to the Z axis, and a contour of a surface of the conductive member 1800 in contact with the electrodes 1802 (see FIGS. 26A to 27B, discussed below).

FIG. 25A illustrates an FSR 1120 disposed on a circuit board 1108, with the conductive member 1800 visible and two electrodes 1802a, 1802b disposed below the conductive member 1800 and spaced apart from one another, as shown in dashed lines. FIG. 25B illustrates an alternative configuration of electrodes 1802c, 1802d on a circuit board 1108, in accordance with some embodiments, with a projection of the conductive member 1800 shown in dashed lines. The electrodes 1802 may be implemented as two rectangular electrodes or other-shaped electrodes 1802a, 1802b offset from one another in the XY plane as shown in FIG. 25A, or as a pair of interdigitated electrodes 1802c, 1802d as shown in FIG. 25B. In the embodiment of FIG. 25A, the size and offset of the electrodes 1802a, 1802b may be selected to achieve a desired relationship between load and change in resistance.

In the embodiment of FIG. 25B, the electrodes 1802c, 1802d may include between 2 and 20 fingers 1804, such as between 10 and 15 fingers 1804. Parameters such as the length (X direction in FIG. 25B), overlapped length, width (Y direction in FIG. 25B), and spacing of the fingers 1804 may be selected to achieve a desired relationship between load and change in resistance. In the embodiment of FIG. 25B, the number of fingers 1804 in contact with the conductive member 1800 as well as the degree to which surface features of the conductive member 1800 are collapsed upon pressing against the fingers 1804 determines the change in resistance to a greater degree than changes in resistance of the bulk of the conductive member 1800 due to compression.

FIGS. 26A to 26C and 27A to 27C illustrate example cross-sectional shapes of the conductive member 1800 in planes parallel to the Z direction. In the embodiment of FIG. 26A, the conductive member 1800 has a substantially cuboid shape, optionally with some surface roughness. In the embodiment of FIG. 26A, changes in resistance may be primarily due to collapsing of surface irregularities as well as increasing the density of the bulk of the conductive member 1800 in response to loading.

In the embodiments of FIGS. 26B and 26C, a lower or electrode-facing surface of the conductive member 1800 includes one or more cylindrical or spherical features 1902. In the embodiments of FIGS. 26B and 26C, changes in resistance may be due to all of (a) collapsing of surface irregularities, (b) increasing the density of the bulk of the conductive member 1800 (and thereby decreasing the distance between conductors in the bulk) in response to loading, and (c) increase in the extent of the features 1902 that are parallel to and in contact with the electrodes. In the embodiment of FIG. 26C, each feature may be positioned over one of the electrodes 1802.

Referring to FIGS. 27A and 27B, the conductive member 1800 may have various other cross-sectional shapes in planes parallel to the Z direction that may be used alone or in combination with any of the cross-sectional shapes of FIGS. 26A to 26C. For example, the conductive member 1800 may have a rectangular cross-sectional shape as shown in FIG. 25A, a circular shape as shown in FIG. 27B, or an annular shape as shown in FIG. 27A. For the embodiments of FIGS. 27A and 27B, the outer diameter may be selected to provide a desired relationship between load and change in resistance. For the embodiments of FIG. 27A, the inner diameter may be additionally or alternatively selected to provide a desired relationship between load and change in resistance. The configuration of any of the compressible members illustrated in FIGS. 26A-26C and FIGS. 27A and 27B, or any combination thereof, can be combined with the electrode configuration described above in connection with FIG. 26A, FIG. 26B, and/or any other suitable electrode configuration.

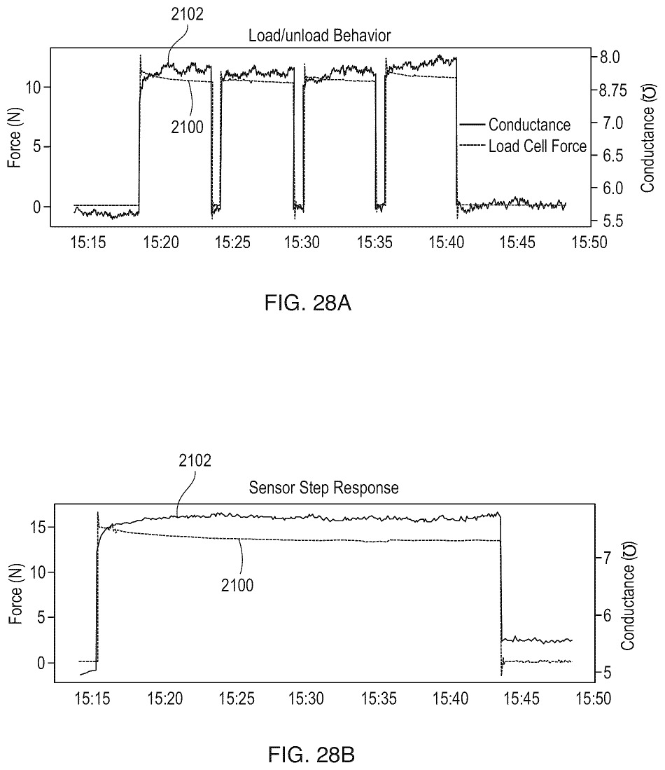

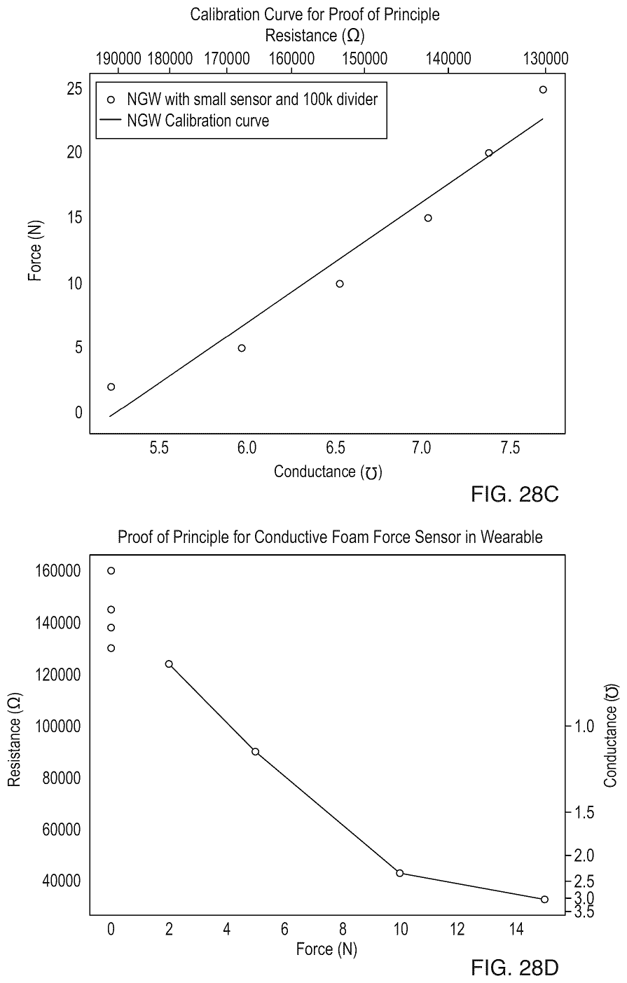

FIGS. 28A to 28D illustrate the change in resistance and conductance of an FSR according to the embodiments disclosed herein. Referring specifically to FIGS. 28A and 28B, many FSRs exhibit a hysteresis such that there may be a lag between application of a load (plot 2100) and a change in conductance (plot 2102). However, in some scenarios, the change in conductance still provides a valuable estimate of load that may be used as the force data 706 as described above. In addition, the known frequency response of the FSR may be used to filter, adjust, or otherwise condition outputs of the FSR to at least partially compensate for the hysteresis.

FIG. 28C illustrates measurements of conductance with respect to loading of an FSR made of a conductive polymer. As is apparent, though not perfectly linear, a clear proportionality is present between force and conductance that can be approximated as linear.

FIG. 28D illustrates measurements of resistance with respect to force for an FSR implemented using a conductive member. As is apparent, for low applied force, e.g., less than 0.2 N, the change in resistance is very large with respect to force and may not be particularly useful for measuring an applied load. However, for applied forces greater than 2 N, a clear proportionality is present between force and resistance. A preload as described above may be used to ensure that the resistance of the FSR is proportional to load. Put another way, a preload as described above may be used to ensure that the FSR is in its active load range even in the absence of an externally-applied compressive load.

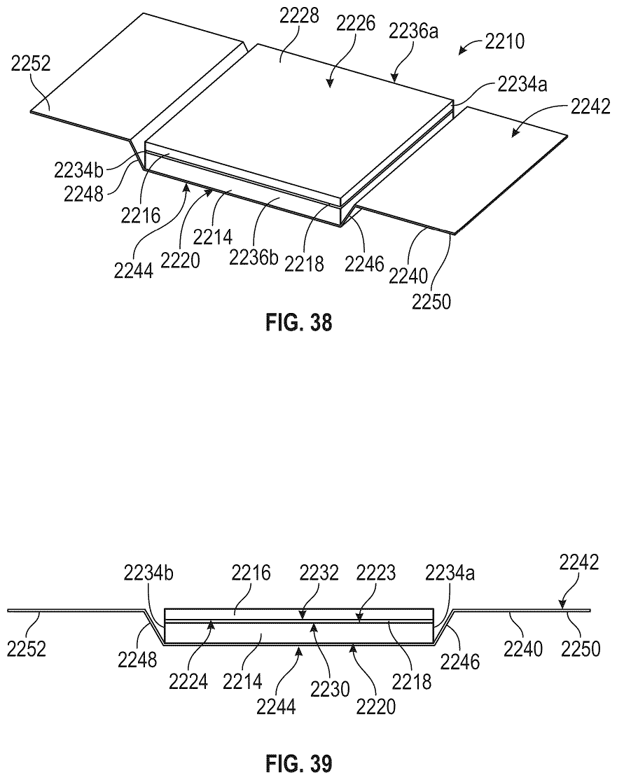

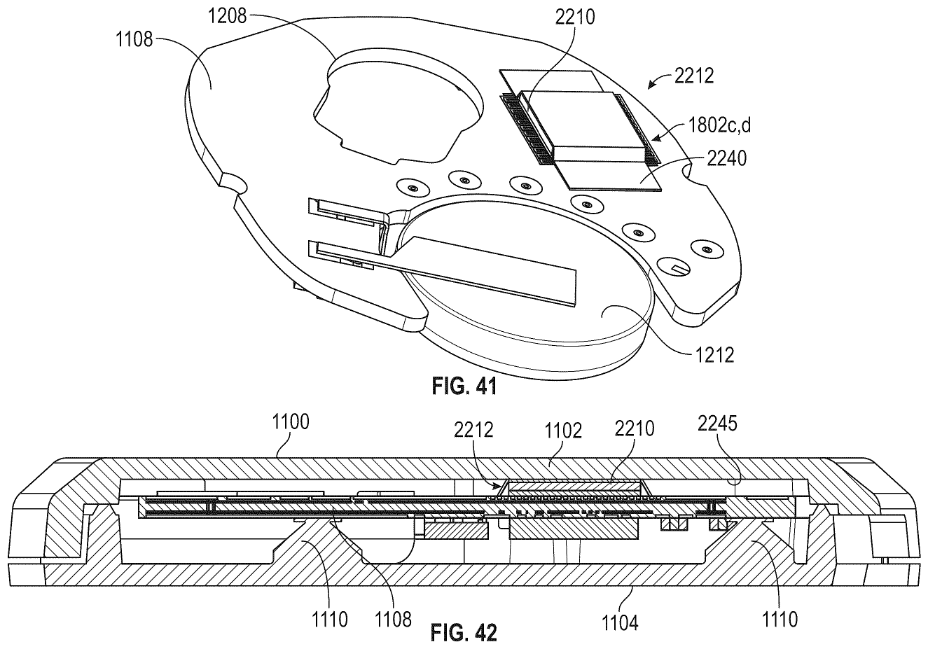

FIG. 38 illustrates a variation in a configuration of a conductive member 2210 for a force sensor or FSR 2212 (marked in FIG. 41) that may be utilized in embodiments herein. FIG. 38 is a perspective lower or distal view of the conductive member 2210 (with the conductive member 2210 being inverted upon assembly within the on-skin wearable medical device as shown in FIG. 41). The FSR 2212 may include the features of other forms of force sensors or FSRs 1120 disclosed herein unless stated overwise. The conductive member 2210 may include a plurality of layers. For example, the conductive member 2210 may include a first layer 2214 and a second layer 2216. The conductive member 2210 may include a third layer 2218 that may be positioned between or sandwiched between the first layer 2214 and the second layer 2216. The layers 2214, 2216, 2218 may each be planar, and may extend in planes that are parallel with each other (although offset from each other at a height).

The first layer 2214 may be a compressible and/or compliant layer. The first layer 2214 may be an elastomer in embodiments and may be a backing layer for the second layer 2216 in embodiments. The first layer 2214 may have a thickness that is greater than the thickness of the second layer 2216 although other configurations (e.g., a same or lesser thickness) may be utilized in embodiments.

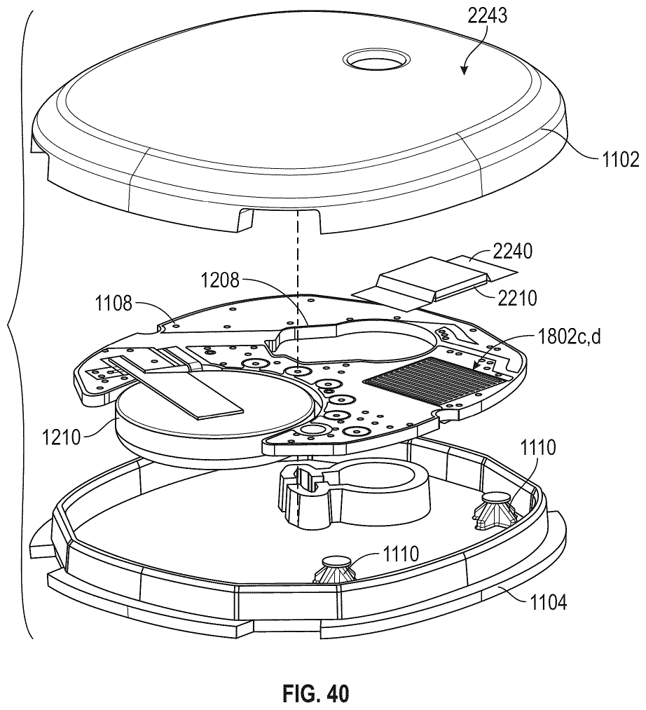

The first layer 2214 may have an upper surface 2220, raised surface, or surface that faces opposite the electrodes 1802c, d (marked in FIG. 40). The first layer 2214 may include a lower surface 2223 (marked in FIG. 39) that faces opposite the upper surface 2220.

The second layer 2216 may be a compressible and/or compliant layer. The second layer 2216 may be a thermoplastic in embodiments, such as polyethylene terephthalate (PET) or another form of thermoplastic as desired. The second layer 2216 may be compliant to conform to a shape of the underlying electrodes 1802c, d upon pressure being applied to the second layer 2216, as discussed in regard to FIGS. 25A-27B. The degree to which the second layer 2216 presses against the electrodes 1802c, d determines the change in resistance of the FSR 2212 (proportionate to the force upon the second layer 2216). A compressibility and/or compliance of the first layer 2214 as a backing layer may enhance the ability of the second layer 2216 to conform to the shape of the underlying electrodes 1802c, d, to thereby change the resistance. An elastomer utilized for the first layer 2214 may increase the sensitivity of the second layer 2216 by allowing the second layer 2216 to deform around the traces of the electrodes 1802c, d, which may increase the surface area of contact (and thus enhance the conductivity and sensitivity of the FSR 2212).

In embodiments, the second layer 2216 may have an upper surface 2224, raised surface, or surface (marked in FIG. 39) that faces opposite the electrodes 1802c, d (marked in FIG. 40). The second layer 2216 may include a lower surface 2226 facing opposite the upper surface 2224, with the lower surface 2226 being the electrode-facing surface of the second layer 2216.

In embodiments, the lower surface 2226 may include a coating or a layer (e.g., a fourth layer of the conductive member 2210) of a force sensitive material. The force sensitive material may be a force sensitive ink for contacting the electrodes 1802c, d (marked in FIG. 40). Other configurations may be utilized in examples.

The third layer 2218 may bond the first layer 2214 to the second layer 2216. The third layer 2218 may include an adhesive for adhering the first layer 2214 to the second layer 2216. The third layer 2218 may comprise the adhesive itself, providing an adhesive layer having an upper surface 2230 and a lower surface 2232. In embodiments, the third layer may comprise a tape that includes adhesive on both sides of the tape for adhering to both the first layer 2214 (the lower surface 2223 of the first layer 2214) and the second layer 2216 (the upper surface 2224 of the second layer 2216). The adhesive of the third layer 2218 (whether disposed on tape or not) may comprise a pressure sensitive adhesive or other form of adhesive as desired. Other forms of bonding of the first layer 2214 to the second layer 2216 may be utilized in embodiments as desired.

The conductive member 2210 may include a first pair of opposed side surfaces 2234a, b, and a second pair of opposed side surfaces 2236a, b. Each of the side surfaces 2234a, b, 2236a, b may extend perpendicular to the planes of the respective lower surface 2226 and upper surface 2220 of the conductive member 2210, or other shapes or configurations may be utilized in examples. The lower surface 2226 and upper surface 2220 of the conductive member 2210 may extend in parallel planes with each other and each may be flat (or may have another configuration as represented in FIGS. 26B-27B as desired). The planar configuration of each of the first layer 2214, second layer 2216, and third layer 2218 may result from a formation process of the conductive member 2210 in embodiments (which may be a reel-to-reel formation process as represented in FIG. 43). The FSR 2212 may be a converted FSR in which components are adhered together utilizing adhesive.

In embodiments, a coupling member may be provided for coupling the conductive member 2210 to a substrate. The substrate may be the substrate that the electrodes 1802c, d are positioned on (e.g., a circuit board 1108 as shown in FIG. 25B). The coupling member may have a variety of forms and may comprise an adhesive tape 2240 in embodiments. The adhesive tape 2240 may comprise a planar body having a lower surface 2242 or substrate facing surface for adhering to the substrate and to the conductive member 2210 (the upper surface 2220 or outer surface of the conductive member 2210). The lower surface 2242 may include an adhesive such as a pressure sensitive adhesive or another form of adhesive as desired. The adhesive tape 2240 may have a central portion 2244, bend portions 2246, 2248 or angled portions, and wing portions 2250, 2252. The adhesive tape 2240 may comprise a polymer tape or may have another configuration in examples.

The central portion 2244 may extend in a parallel plane with the upper surface 2220 or outer surface of the conductive member 2210 and may cover and adhere to the upper surface 2220 (which is positioned opposite the electrodes 1802c, d). The bend portions 2246, 2248 may bend from the central portion 2244 to the wing portions 2250, 2252 and may position the wing portions 2250, 2252 at a different height than the central portion 2244 (e.g., closer to the substrate or circuit board 1108). The bend portions 2246, 2248 may cover the side surfaces 2234a, b of the conductive member 2210.

The wing portions 2250, 2252 may protrude outward from the respective bend portions 2246, 2248 and may be configured to adhere to the substrate at the sides of the conductive member 2210. The wing portions 2250, 2252 may be planar or may have another shape as desired. The width of the wing portions 2250, 2252 from outer end to outer end may be between 6 millimeters to 10 millimeters (e.g., 7 millimeters), and the width of the conductive member 2210 may be between 2 millimeters and 5 millimeters (e.g., 3 millimeters). Other sizes or proportions of sizes may be utilized in embodiments as desired.

In embodiments, the adhesive tape 2240 and conductive member 2210 may comprise a unit that is formed prior to coupling to the substrate that includes the electrodes 1802c, d. The components of the adhesive tape 2240 and conductive member 2210 may be coupled together in a configuration as represented in FIGS. 38 and 39 and may then be coupled to the substrate (e.g., to the circuit board 1108, with the conductive member 2210 in alignment with the electrodes 1802c, d). Other methods may be utilized in embodiments (e.g., the conductive member 2210 may be placed upon the substrate in alignment with the electrodes 1802c, d and then the adhesive tape 2240 may be applied to the conductive member 2210, among other methods).

FIG. 40 illustrates an assembly view of the FSR 2212 (marked in FIG. 41) and other components of the wearable device. The conductive member 2210 and coupling member in the form of the adhesive tape 2240 are shown positioned above the electrodes 1802c, d of the FSR 2212. The unit comprising the conductive member 2210 and adhesive tape 2240 is placed upon the electrodes 1802c, d, with the adhesive tape 2240 coupling the conductive member 2210 to the circuit board 1108 at the sides of the conductive member 2210.

The conductive member 2210 may be positioned upon the circuit board 1108 with the conductive member 2210 disposed between the circuit board 1108 and the upper housing 1102. As such, a force applied to the upper housing 1102 in the normal direction or Z-direction as disclosed herein will press the conductive member 2210 towards the electrodes 1802c, d for operation of the FSR 2212. The first layer 2214 or elastomer layer of the conductive member 2210 accordingly may be positioned between the second layer 2216 (marked in FIG. 38) and the upper surface 2243 of the housing 1100. The first layer 2214 is configured to receive a force that is applied to the upper surface 2243 to vary the pressure-sensitive conductance of the FSR 2212.

In embodiments, one or more of the first layer 2214 or the second layer 2216 is compressible, to receive the compressive force applied to the upper housing 1102. Further, a concentrator as disclosed herein may not be utilized, or may be utilized as desired. The supports 1110 as disclosed herein may be utilized, and may apply a preload to the FSR 2212 as desired. Other forms of supports as disclosed herein may be utilized as desired, or the supports may be excluded in embodiments.

FIG. 41 illustrates an assembled view of the FSR 2212 upon the circuit board 1108. Other forms of electrodes (e.g., electrodes 1802a, b shown in FIG. 25A, among others) may be utilized with the FSR 2212 as desired.

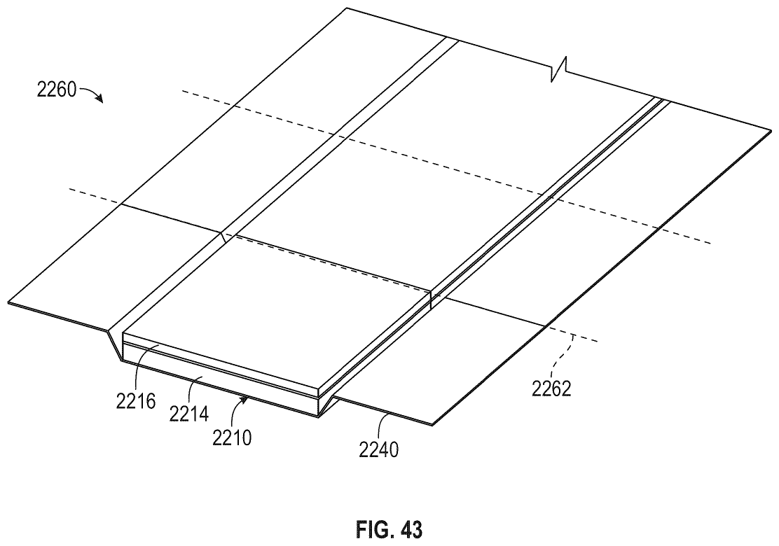

FIG. 42 illustrates a side cross sectional view of the FSR 2212 within the housing 1100. The unit comprising the conductive member 2210 and the adhesive tape 2240 may be in direct contact with an interior surface 2245 of the upper housing 1102 that faces opposite the upper surface 2243 of the upper housing 1102. As such the force applied to the upper housing 1102 may be applied to the conductive member 2210 in a direction towards the electrodes 1802c, d.

Other positions of the FSR 2212 may be utilized as disclosed herein.

FIG. 43 illustrates a schematic of an exemplary manufacturing process for the unit comprising the conductive member 2210 and the adhesive tape 2240. The unit may be formed in reel-to-reel processing in embodiments. For example, reel-to-reel processes may be utilized to layer the first layer 2214 and the second layer 2216 together. Elongate sheets of materials including the first layer 2214 and second layer 2216, respectively, may be bonded together via the third layer 2218 (marked in FIG. 38) in reel-to-reel processing. The third layer 2218 may be bonded to the first layer 2214 or second layer 2216 initially, and then correspondingly bonded to the other layer (either the other of the second layer 2216 or the first layer 2214). Alternatively, the layers 2214, 2216, 2218 may be bonded together simultaneously. Similarly, an elongate sheet of material including the adhesive tape 2240 may be bonded to the first layer 2214 in reel-to-reel processing. Bend portions in the elongate sheet including the adhesive tape 2240 may be formed. Alternatively, the adhesive tape 2240 may be initially bonded to the first layer 2214. A resulting elongate sheet 2260 of materials including the adhesive tape 2240 and the conductive member 2210 is shown in FIG. 43. The elongate sheet 2260 may comprise a continuous elongate body formed to decrease manufacturing cost and complexity.

The unit comprising the conductive member 2210 and the adhesive tape 2240 may be singulated from the elongate sheet 2260 of materials. For example, the unit may be cut along a cut line 2262 that may singulate the unit and form the opposed side surfaces 2236a, b (marked in FIG. 38). The cut line 2262 may extend perpendicular to the length of the elongate sheet 2260. Successive cuts may be made to iteratively cut units from the elongate sheet 2260 as desired (as marked with other dashed lines in FIG. 43). The units may be cut from the elongate sheet 2260 having multiple of the units therein. The singulated units may be applied to the circuit board 1108 as represented in FIG. 40. As such, improved ease and speed of manufacture of the unit may be provided.

Variations in the method may manufacture may be utilized. Other methods of manufacture may be utilized in embodiments.

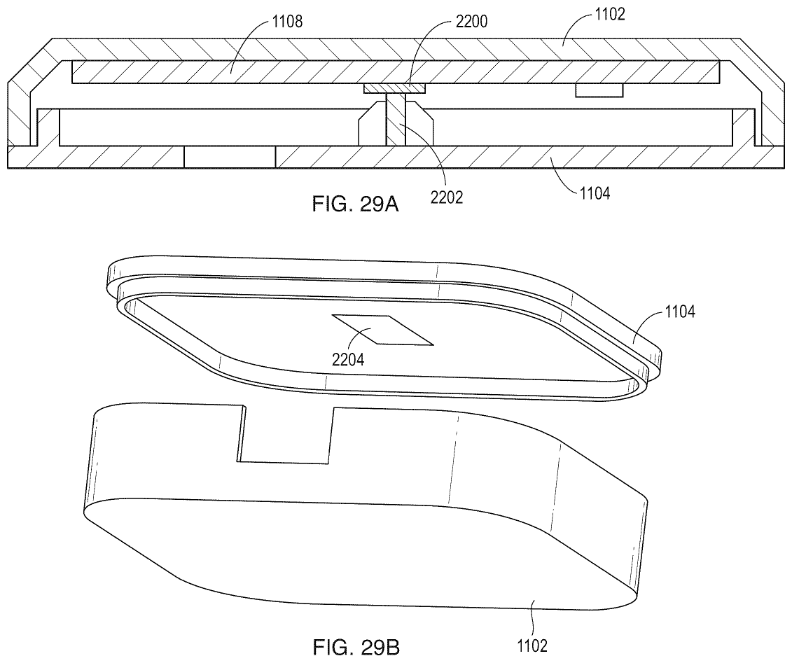

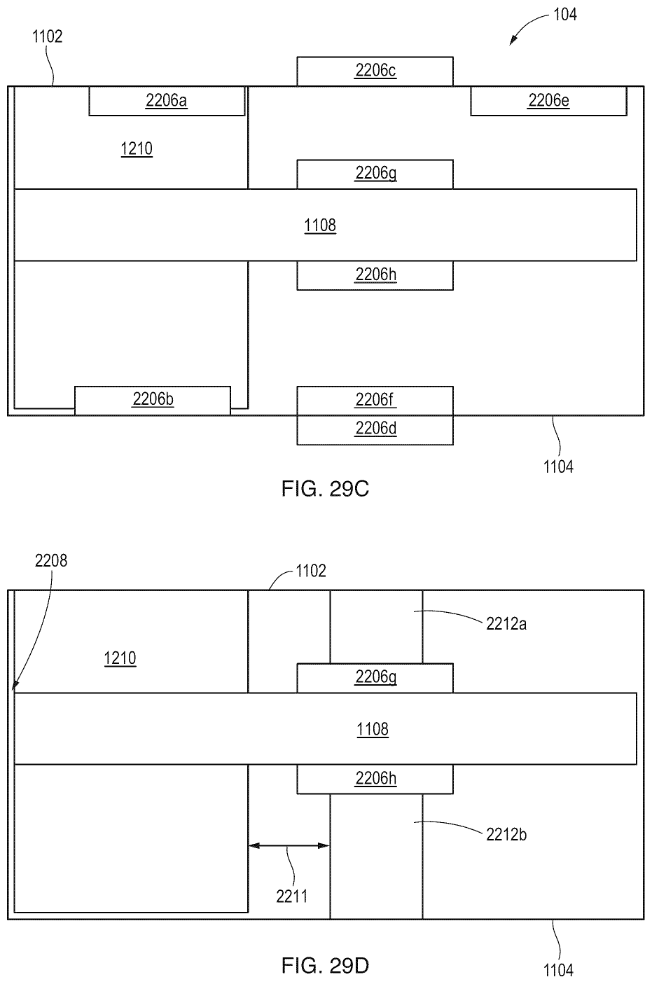

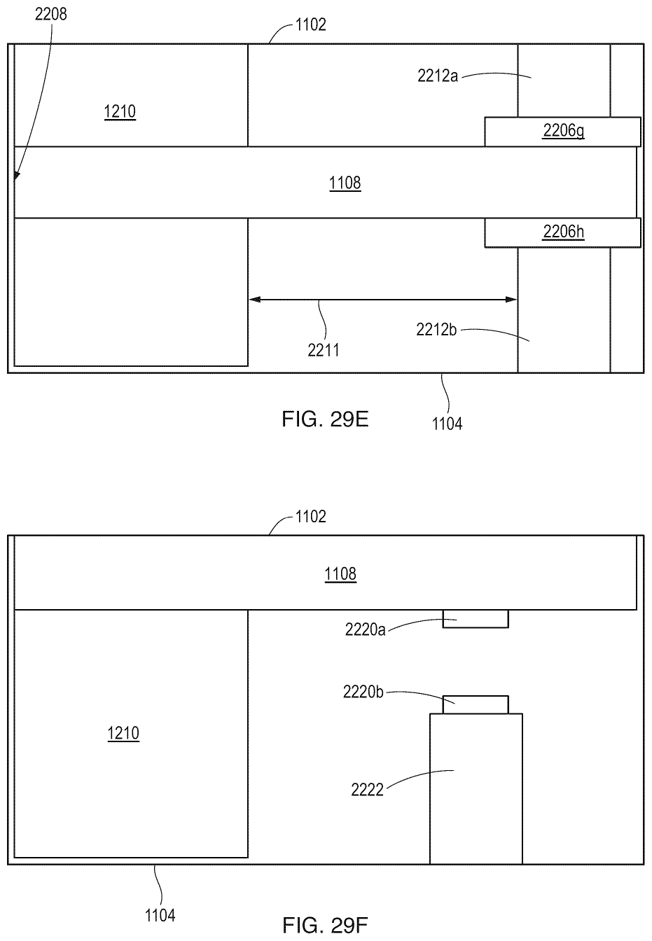

Referring to FIGS. 29A to 29F, various alternative approaches for sensing load on a continuous analyte sensor system 104 (and/or on an on-skin or wearable device thereof) may also be implemented. For example, referring to FIG. 29A, which shows a schematic cross-sectional view of an on-skin device, in some embodiments, an FSR 2200 may be interposed between a support 2202 and the circuit board 1108, with the support 2202 secured to the lower housing 1104. The embodiment of FIG. 29A may have the advantage of the FSR 2200 being secured to the same side of the circuit board 1108 as other components thereby simplifying manufacture. The support 2202 may be implemented as a support according to any of the supports 1110, 1400, 1500, 1600 described above and/or as a compressible member such as a structural foam, polymer, or elastomeric member.

The support 2202 may be sized and/or shaped to preload the FSR 2200 enough to bring the FSR into its active load range, without application of any external load. Likewise, the support 2202 may be made compliant (see, e.g., the embodiments of FIGS. 21A to 23B) to provide a proportional change in resistance for a desired range of loads. The compliant supports may either be inserted during assembly as separate parts from the housing or may be molded in a multi-shot molding operation to the housing. A compliant support may be chosen instead of using the material of the housing (or other rigid or semi-rigid material) to reduce the stress on the circuit board, the housing, or both while still allowing the FSR to sense externally-applied loads.

Referring to FIG. 29B, which shows a schematic illustration of another on-skin device, in some embodiments, a force sensor may be implemented as a strain gauge 2204. For example, the strain gauge 2204 may be mounted to the lower housing 1104, as shown, or be mounted to the upper housing 1102 or circuit board 1108. In some embodiments, a strain gauge 2204 may be mounted to a support supporting a circuit board 1108. In these and other embodiments, flexing of the lower housing 1104, upper housing 1102, and/or circuit board 1108 induced by loading will induce strain and a change in resistance of the strain gauge 2204.

FIG. 29C schematically illustrates a plurality of locations 2206a-2206h at which a force sensor (FSR, strain gauge, etc.) may be placed within a continuous analyte sensor system 104. For example, a force sensor may be placed at one or more of the following positions:
Position 2206a between the battery 1210 and the upper housing 1102
Position 2206b between the battery 1210 and the lower housing 1104.
Position 2206c on an external surface of the upper housing 1102.
Position 2206d on an external surface of the lower housing 1104
Position 2206e on an internal surface of the upper housing 1102.
Position 2206f on an internal surface of the lower housing 1104
Position 2206g on an upper surface of the circuit board 1108.
Position 2206h on a lower surface of the circuit board 1108.

Referring to FIGS. 29D and 29E, for positions 2206g and 2206h, the circuit board 1108 may be mounted to other structure in the housing 1100 as a cantilever, for example at a point 2208 on the circuit board 1108, with the remainder of the circuit board 1108 being unsupported other than through the force sensor. A distance 2211 of the positions 2206g, 2206h from the point 2208 may be selected to control the resistance of the circuit board 1108 to deflection. One or more supports 2212a, 2212b may extend between the upper housing 1102 and the position 2206g and/or between the lower housing 1104 and the position 2206h. Accordingly, loading of the upper housing 1102 and/or lower housing 1104 will be transferred by one or both supports 2212a, 2212b to force sensors at positions 2206g and/or 2206h.

Referring to FIG. 29F, in some embodiments, binary or other non-proportional sensing of force may be used. For example, a force sensor may be implemented by a pair of contacts 2220a, 2220b that are spaced apart from one another in a resting state (absent any external loading) and brought into contact in response to loading exceeding a threshold. Once in contact, the contacts 2220a, 2220b may provide a constant low resistance or resistance that decreases further in proportion to further loading up to saturation. In the illustrated embodiment, a first contact 2220a is mounted to the circuit board 1108 and a second contact 2220b is mounted to the lower housing 1104, such as to a support 2222 extending upward from the lower housing 1104. Accordingly, upon loading of the upper housing 1102, the circuit board 1108 will deflect downward and eventually cause the contacts 2220a, 2220b to connect and close a circuit, which may then be sensed and interpreted as force exceeding a force threshold. For example, closing of the circuit may be interpreted as an acute compression event as described above.

### Force Deflectors and Housings for Preventing Compression Events

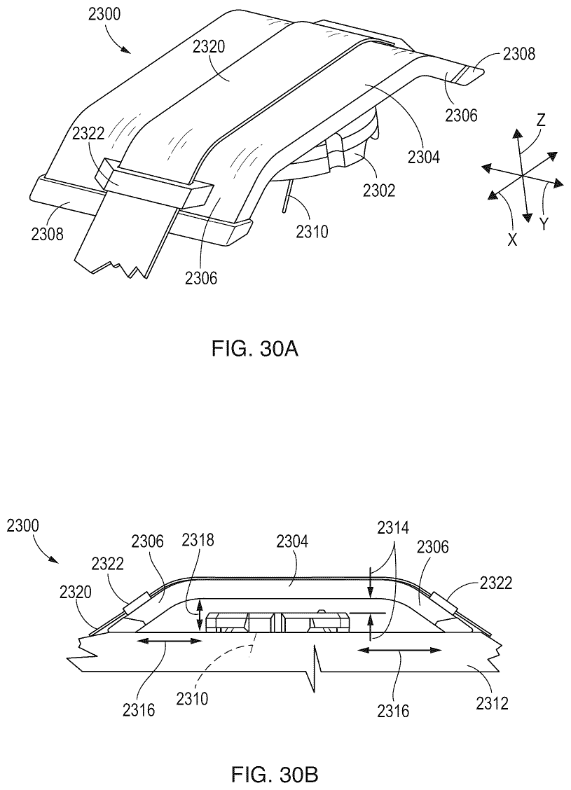

Referring to FIGS. 30A to 37B, in some implementations of the continuous analyte monitoring system 104, various approaches may be used to reduce changes to concentrations of one or more analytes in the region of an analyte electrode 302, 304 resulting from compression events. The approaches of any of FIGS. 30A to 37B may be used either with or without any of the approaches described above with respect to any of FIGS. 5 to 29F or FIGS. 38 to 43.

Referring specifically to FIGS. 30A and 30B, a force deflector 2300 may be used to direct loads outwardly from a housing 2302 or on-skin device of the continuous analyte monitoring system 104. The housing 2302 may have some or all of the attributes of a housing 1100 as described above or have a different configuration.

The force deflector 2300 may include a central portion 2304 that has an extent in the X and Y directions that is at least as great as or greater than that of the housing 2302, such as at least 1.5 times or at least two times dimensions of the housing 2302 in the X and/or Y directions. The force deflector 2300 further includes at least two legs 2306 that extend downwardly from the central portion 2304 in the Z direction. The at least two legs 2306 may be straight and angled with respect to the central portion 2304, as in the illustrated embodiment, or curved. In the illustrated embodiment, a distal portion of each leg 2306 includes a pad 2308. The pad 2308 may be made of a softer material than the material used to form the central portion 2304 and legs 2306 in order to enhance comfort of the user and avoid force concentrations. The central portion 2304 and/or legs 2306 may be made of a flexible material to avoid force concentrations on the skin 2312 of the user. As used herein, the term “flexible” may be defined as having a hardness less than or equal to 50 Shore A, less than or equal to 30 Shore A, or less than or equal to 20 Shore A.

Referring specifically to FIG. 30B, the force deflector 2300 defines various gaps with respect to the housing 2302 and a wire 2310 including sensor electrodes that is embedded in the skin 2312 of the user in use. A vertical gap 2314 along the Z direction may be present between the housing 2302 and the underside of the central portion 2304. The vertical gap 2314 may be from 2 to 10 mm, such as from 5 to 10 mm, or some other value selected to be larger than displacement of the central portion 2304 when subject to loads expected during use. The displacement of the central portion 2304 may be a result of some or all of deflection of the central portion 2304, deflection of the legs 2306, deflection of the pads 2308, and deflection of the skin 2312 supporting the pads 2308.

Horizontal gaps 2316 may be present between the housing 2302 and a closest point of contact between each pad 2308 and the skin 2312. For example, the horizontal gaps 2316 may be equal to between 0.25 and 1 times the largest dimension of the housing 2302 along the X direction. In some embodiments, the horizontal gaps 2316 are between 2 and 30 mm, such as between 10 and 20 mm, along the X direction. With reference to FIG. 30B, the X direction may be defined as the direction along which the legs 2306 are offset from one another.

In the illustrated embodiment, the force deflector 2300 is open-sided. For example, “open-sided” may mean that, within the area spanned by the horizontal gaps 2316 and the extent of the housing 2302 in the X direction, no portion of the force deflector 2300 is in contact with the skin 2312 of the user absent a load on the force deflector 2300. Alternatively, “open sided” may mean that no portion of the central portion 2304 is in contact with the skin 2312 of the patient and no portion of the central portion 2304 will contact the skin 2312 of the patient when loaded less than an amount of a force required to force the central portion 2304 into contact with the housing 2302. Stated in yet another way, “open sided” may mean that no portion of the central portion 2304 is in contact with the skin 2312 of the patient and no portion of the central portion 2304 will contact the skin 2312 of the patient when loaded less than an amount of force required to deflect the central portion 2304 along the Z direction a distance 2318 defined as the separation between the skin 2312 and a center of the central portion 2304 in the absence of loading of the force deflector 2300. In some embodiments, “open sided” may be defined as the central portion 2304 having a flat underside and the legs 2306 having a constant cross section in planes parallel to the Z and X directions along substantially the entire (e.g., at least 90 percent of) the length thereof in the Y direction. Stated yet another way, “open sided” can mean that the skin-contacting portions of the force deflector (pads 2308, in the illustrated embodiment) are spaced apart radially from one another about the on-skin device (while also being spaced apart laterally from the on-skin device) and therefore do not completely encircle the on-skin device. In embodiments, as shown in FIG. 30A, a force deflector can include two skin-contacting portions disposed on opposing sides of the on-skin device. In other embodiments, a force deflector can include three, four, or more skin-contacting portions which are spaced apart radially from one another about the on-skin device, whether symmetrically or non-symmetrically.

The open-sided configuration of the force deflector 2300 ensures that blood and lymphatic fluid can flow between tissue surrounding the wire 2310 and parts of the body outside of the area under the force deflector 2300. Accordingly, measured concentrations of analytes will therefore either be (a) unaffected by compression events, (b) have a higher likelihood of being compensatable, such as using the approach described with respect to FIGS. 5 to 10, and/or (c) less likely to mistakenly trigger an alert resulting from compression.

The force deflector 2300 may secure to the user in various ways. For example, a strap 2320 may pass over the force deflector 2300 and around an arm, leg, or waist, of a patient in order to hold the force deflector 2300 in place. The strap 2320 may secure to itself to form a loop by means of a knot, hook-and-loop fastener, button, clip, buckle, zipper, or other selective fastener. The strap 2320 may be formed into a loop, such as by stitching or adhesive and be made of an elastic material that can be stretched over an arm, leg, or waist of a patient. The force deflector 2300 may define one or more guides 2322 through which the strap 2320 passes and maintain the position of the strap 2320. In some embodiments, the strap 2320 or other structure may be configured to adhere to the skin 2312 of the user and to the force deflector 2300. Note that the housing 2302 may secure to the skin 2312 of the user independently of the force deflector 2300, such as by means of an adhesive or adhesive patch.

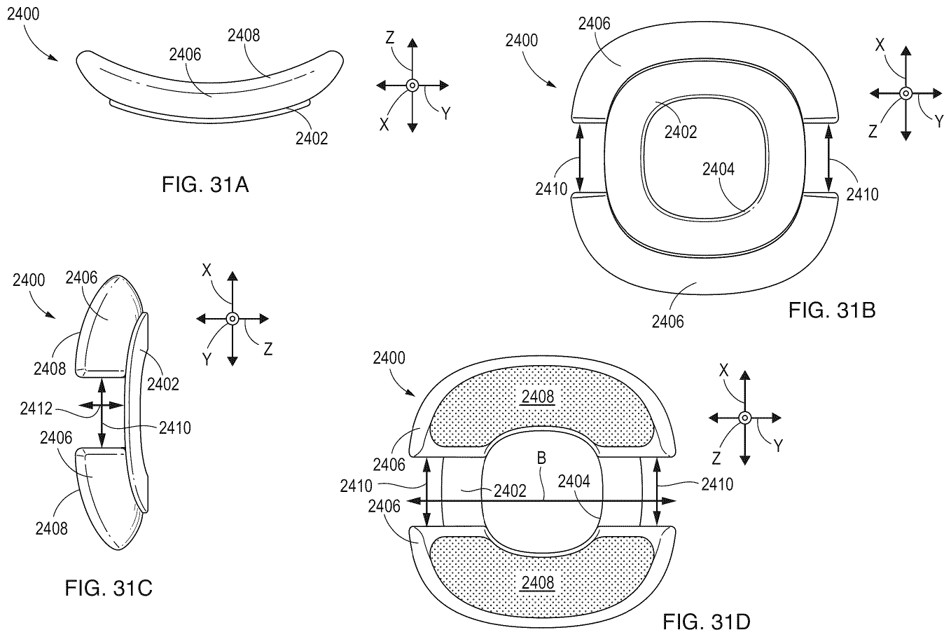

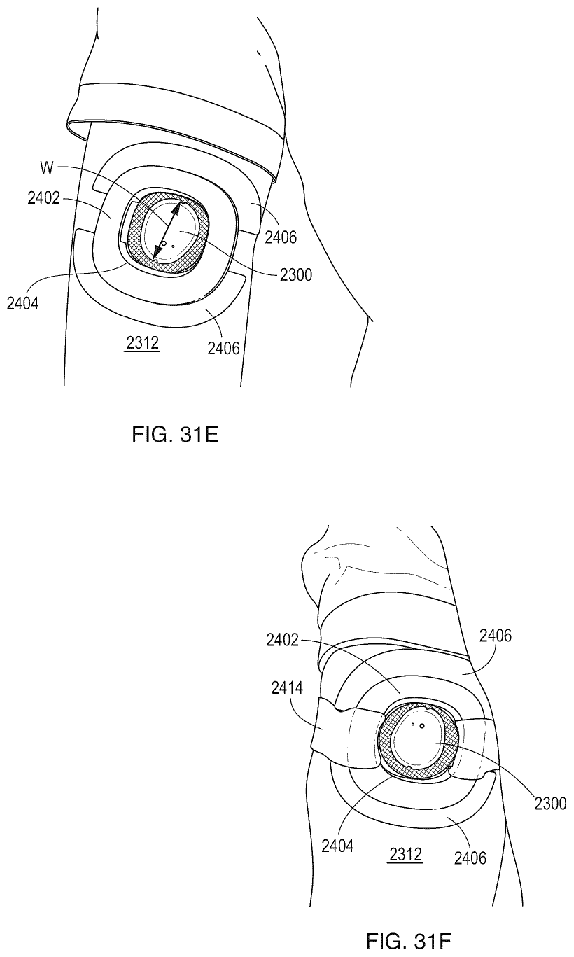

FIGS. 31A to 31F illustrate an alternative force deflector 2400 that may be used to direct loads outwardly from a housing 2302 of the continuous analyte monitoring system 104. The force deflector 2400 may include a central portion 2402 defining an opening 2404. The opening 2404 may have an extent in the X and Y directions during use that is greater than those of the housing 2302. Accordingly, compression of the force deflector 2400 itself toward the skin 2312 of the user will not bring a lower surface of the central portion 2402 into contact with the housing 2302, thereby avoiding transfer of the compressive load to the housing 2302. Although focused loads smaller than the opening 2404 may reach the housing 2302, typical loads resulting from sleeping on, sitting on, or leaning against the force deflector 2400 will not reach the housing 2302. In some embodiments, the opening 2404 may be covered by a mesh or other layer preventing objects from contacting the housing 2302.

The central portion 2402 may be mounted to one or more base portions 2406. Each base portion 2406 has a bottom surface 2408 that rests on the skin 2312 of the user. The base portion 2406 may be relatively wide to distribute forces transferred from the central portion 2402 over the skin 2312. For example, a width W may be defined as the widest dimension of the housing 2302, such as in the XY plane. The width B of the base portions 2406, such as measured along a line extending from a center C of the opening 2404 may be at least 0.25 W, at least 0.3 W, at least 0.4 W, at least 0.5 W or at least 0.6 W along at least 70 percent of the angular extent of the base portion about the center C of the opening 2404.

In some embodiments, the base portions 2406 are made of a softer material than the central portion 2402 to enhance user comfort and reduce force concentrations. For example, the base portions 2406 may be made of a material having a hardness of less than or equal to 50 Shore A, less than or equal to 30 Shore A, or less than or equal to 20 Shore A. The central portion 2402 may have a hardness of greater than 30 Shore D, greater than 50 Shore D, or greater than 80 Shore D.

Bottom surfaces 2408 of the base portions 2406 that contact the skin 2312 of the user may be textured, or coated with material (e.g., soft fabric). The bottom surfaces 2408 may include an adhesive or adhesive layers to temporarily secure the base portions 2406 to the skin 2312 of the user (see FIG. 31E).

The bottom surfaces 2408 may be curved in one or more planes in order to conform to the curvature of an arm, leg, torso, or other portion of the body of the user as best seen in FIGS. 31A and 31C, which show end, top plan, and side views, respectively, of the force deflector 2400. In some embodiments, the central portion 2402 may also be curved in one or more planes in order to approximate the curvature of the portion of the body of the user to which the force deflector 2400 is secured.

The base portions 2406 may be spaced apart from one another by gaps 2410 in the XY plane. The gaps 2410 facilitate the flow of blood and lymphatic fluid during compression events. The width of the gaps 2410 may be at least 0.4 W, at least 0.5 W, at least 0.6 W, or at least 0.7 W.

An additional gap 2412 may be present between the central portion 2402 and the skin 2312. The gap 2412 may be selected such that the central portion 2402 does not contact the skin 2312 during compression events, whether as a result of compression of the base portions 2406, deflection of the skin 2312, and/or bending of the central portion 2402.

Referring to FIG. 31F, the force deflector 2400 may secure to the arm, leg, or waist of the user using a strap 2414. For example, the strap 2414 may pass into the gaps 2410 and encircle portions of the central portion 2402 spanning the gaps 2410 and encircle the arm, leg, or waist of the user.

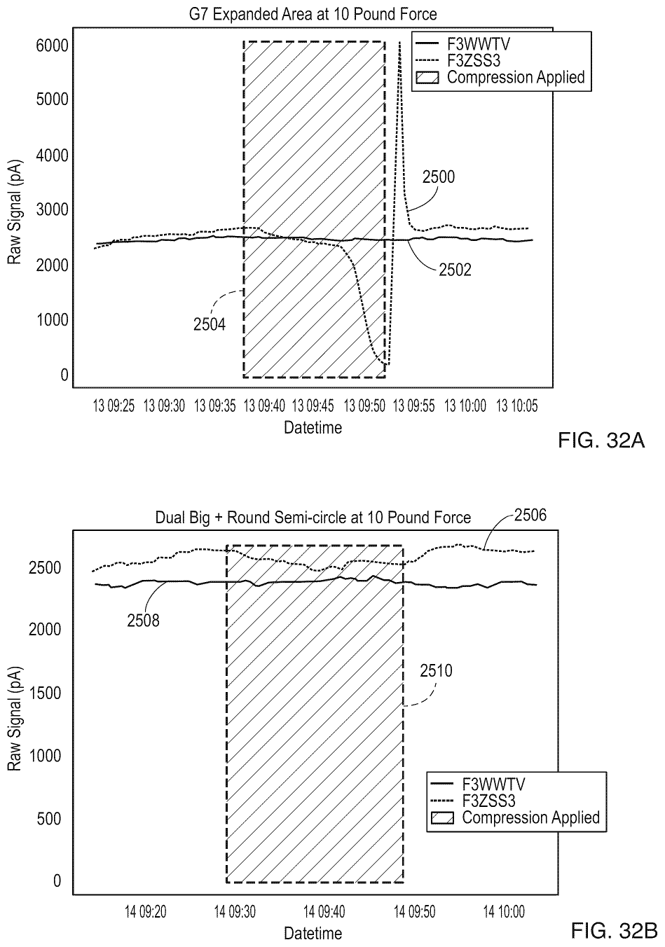

FIGS. 32A and 32B illustrate the benefit of a force deflector 2400. FIG. 32A includes a plot 2500 of current corresponding to glucose concentration during a compression event and a plot 2502 of current corresponding to glucose concentration in the absence of a compression event. The measurements of plot 2500 were measured during a compression event 2504 with a 10 pound force applied to the continuous analyte monitoring system 104 without the benefit of a force deflector 2400.

FIG. 32B includes a plot 2506 of current corresponding to glucose concentration measured during a compression event and a plot 2508 of current corresponding to glucose concentration in the absence of a compression event. The measurements of plot 2506 were measured during a compression event 2510 with a 10 pound force applied to the continuous analyte monitoring system 104 through a force deflector a force deflector 2400. As is readily apparent, the sharp drop and spike in the plot 2500 is not present in the plot 2506. In practice, the force deflector 2400 would therefore prevent an unnecessarily alert signaling a hypoglycemic event.

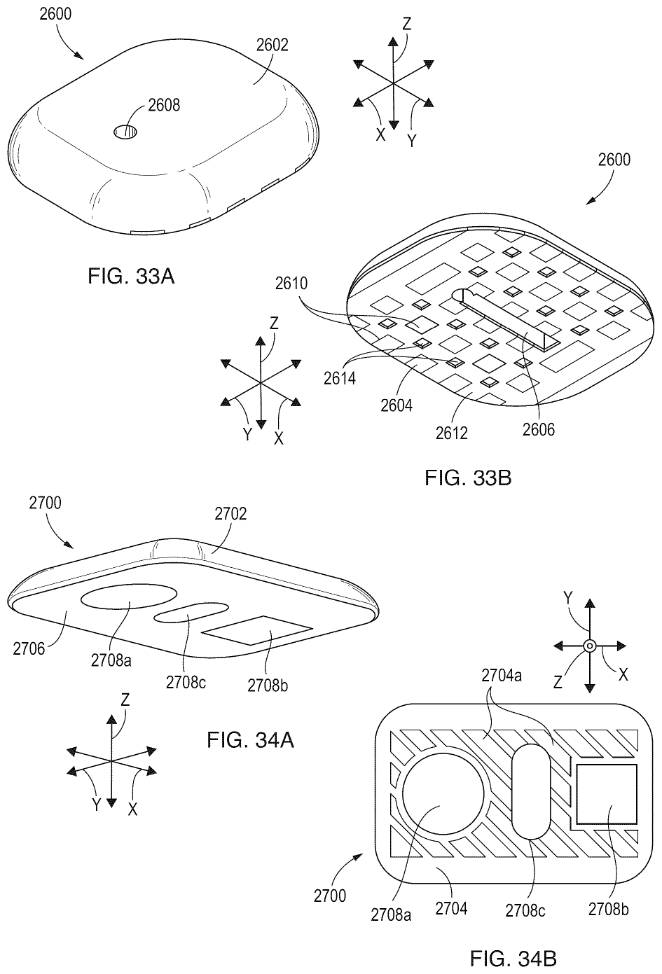

FIGS. 33A to 35B illustrate additional configurations of the continuous analyte monitoring system 104 which can serve to reduce transmission of compressive loads to tissue surrounding one or more electrodes 302, 304 thereof. The configurations of FIGS. 33A to 35B may be used alone or in combination with any of the features described above with respect to FIGS. 5 to 32B or FIGS. 38 to 43.

Referring specifically to FIGS. 33A and 33B, in some embodiments, the continuous analyte monitoring system 104 includes a housing 2600 including an upper housing 2602 and a lower housing 2604, the lower housing 2604 interfacing with the skin of the user during use, for example through an adhesive layer or patch. The lower housing 2604 defines a pocket or slot 2606 for receiving a sensor and the upper housing 2602 defines an opening 2608 for receiving a needle used to insert the sensor into the skin of the user. A circuit board and battery as for other embodiments disclosed herein may be mounted within the housing 2600 between the upper housing 2602 and lower housing 2604 and coupled to the sensor in any suitable fashion, for example as described for other embodiments disclosed herein.

In the illustrated embodiment, the lower housing 2604 includes a grid of channels 2610 that are at least partially filled with a flexible lining 2612, which can comprise a flexible material such as a flexible polymer. Within the lining 2612, a plurality of openings 2614 may be formed. The openings 2614 may be empty or filled with a material that is softer than that forming the flexible lining. The flexible lining 2612, and possibly the openings 2614, can help to absorb and/or displace compressive loads applied to the upper housing and thereby facilitate the flow of blood and lymphatic fluid during compression events.

The lower housing 2604 may be formed of a more rigid material than the flexible lining 2612. For example, the flexible lining 2612 may be made of a material having a hardness of less than or equal to 50 Shore A, less than or equal to 30 Shore A, or less than or equal to 20 Shore A. The lower housing 2604, and possibly the upper housing 2602, may have a hardness of greater than 30 Shore D, greater than 50 Shore D, or greater than 80 Shore D.

The compression of the skin of the wearer during use is limited to small concentration zones where the rigid portions of the lower housing 2604 are in contact with the skin. In between these concentration zones, in the regions of the flexible liner 2612 and/or the openings 2614, are relatively less compressed or uncompressed areas that can be controlled by the design of the channels 2610 on the lower housing 2604 and/or the openings 2614 in the lining 2612. The material properties of the flexible lining 2612 (if any) and the presence of openings 2614 can allow for expansion of the flexible lining in a direction parallel to the skin. These relatively less compressed or uncompressed zones can act as mass transfer channels that ensure flow of glucose and oxygen to the sensor insertion site.

Referring specifically to FIGS. 34A and 34B, in some embodiments, the continuous analyte monitoring system 104 includes a housing 2700 including an upper housing 2702 and a lower housing 2704, the lower housing 2704 interfacing with the skin of the user during use, for example through an adhesive layer or patch. The upper housing 2702 may define an opening for receiving a needle used to insert the sensor into the skin of the user.

A circuit board 2706 is mounted within the housing 2700 and may be implemented as a flexible printed circuit board (PCB). The circuit board 2706 is therefore flexible other than at the mounting locations of rigid components, such as the battery 2708a, integrated circuit 2708b, and sensor pocket 2708c. The battery 2708a and integrated circuit 2708b may be coupled to a sensor positioned within the sensor pocket 2708c as for other embodiments disclosed herein. The lower housing 2704 may be made of a flexible polymer and may include one or more cutouts 2704a. The cutouts 2704a may be oriented to allow for preferential expansion of the flexible polymer in a direction parallel to the surface of the skin of the user.

The embodiment of FIGS. 34A and 34B may advantageously be configured to localize compressive forces to spaced-apart portions of the lower housing, as opposed to spreading them the full cross-sectional area of the lower housing 2704 in the XY plane. A combination of flexible and rigid regions in the housing can help allow mass flow of blood and lymphatic fluid very close to the sensor insertion site. The rigid components, such as the battery 2708a and integrated circuit 2708b, may be offset form the sensor pocket 2708c in the XY plane, such as by between 2 and 10 mm, to further reduce the transmission of force close to the insertion site. In some embodiments, the upper housing 2702 itself may be made of a flexible material that can bend substantially, at least in regions between the rigid components of the device, which allows for additional load at different sides of the housing 2700 without transferring such loads over the entire extent of the housing 2700 in the XY plane.

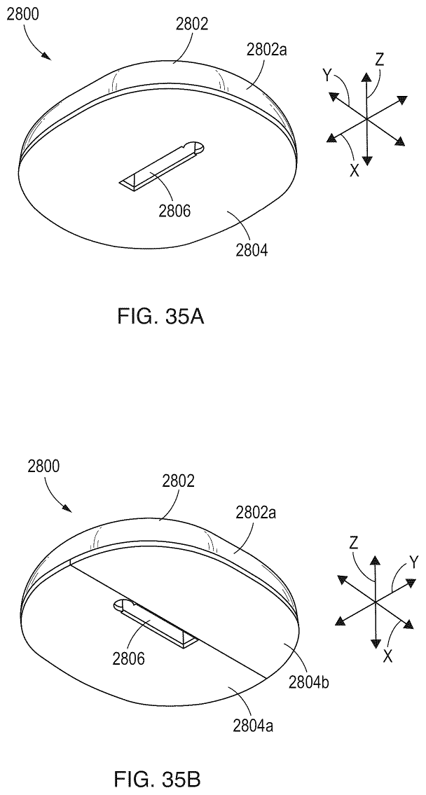

Referring specifically to FIGS. 35A and 35B, in some embodiments, the continuous analyte monitoring system 104 includes a housing 2800 including an upper housing 2802 and a lower housing 2804, the lower housing 2804 interfacing with the skin of the user during use. The lower housing 2804 defines a recess or slot 2806 for receiving a sensor. The upper housing 2802 may define an opening for receiving a needle used to insert a portion of the sensor into the skin of the user.

The upper housing 2802 may include downwardly extending sidewall 2802a with the lower housing 2804 extending inwardly from the sidewall 2802a such that the upper housing 2802 and lower housing 2804 define a volume for containing a battery, integrated circuit, printed circuit board, or other components of the continuous analyte sensor system 104.

In the embodiment of FIGS. 35A and 35B, the upper housing 2802 may be made of a rigid material whereas at least a portion of the lower housing 2804 interfacing with the skin of the user during use may be made of a flexible material. For example, the flexible material may have a hardness of less than or equal to 50 Shore A, less than or equal to 30 Shore A, or less than or equal to 20 Shore A. The rigid material may have a hardness of greater than 30 Shore D, greater than 50 Shore D, or greater than 80 Shore D. Components such as a circuit board, integrated circuit, and battery may be mounted to the upper housing 2802 and offset from the flexible material of the lower housing 2804. As shown in FIG. 35A, the flexible material of the lower housing 2804 may extend across substantially all (e.g., at least 90 percent) of an area within the sidewall 2802a. The flexibility of the lower housing 2804 may advantageously permit transport of blood and lymphatic fluid near the insertion site even during compression events.

As shown in FIG. 35B, in certain other embodiments, the lower housing 2804 includes a first portion 2804a and a second portion 2804b. The first portion 2804a may be rigid, such as being made of the same material as the upper housing 2802. The second portion 2804b is made of a flexible material. In some embodiments, the slot 2806 for receiving the sensor is positioned at a boundary between the first portion 2804a and the second portion 2804b. In some embodiments, each of the first portion 2804a and the second portion 2804b occupies at least 40 percent or at least 45 percent of the area within the sidewall 2802a in the XY plane. The embodiment of FIG. 35B may have the advantage of allowing transport of blood and lymphatic fluid near the insertion site even during compression events. The rigid first portion 2804a transfers force from the upper housing 2802 to the skin 2312 of the user facilitating reduction in loading of the second portion 2804b. In either of the embodiments of FIGS. 35A and 35B, the flexible material of the lower housing 2804 may include cutouts or recesses to further promote the transport of blood and lymphatic fluid.

In the embodiments of FIGS. 35A and 35B, the flexible material forming the lower housing 2804 or second portion 2804b may be formed by molding a flexible and/or compressible material such as foam over and/or into the upper housing 2802 and components positioned therein (battery, integrated circuit, circuit board, etc.). The compressible material used may form a skin on an external or skin-facing surface of the wearable device.

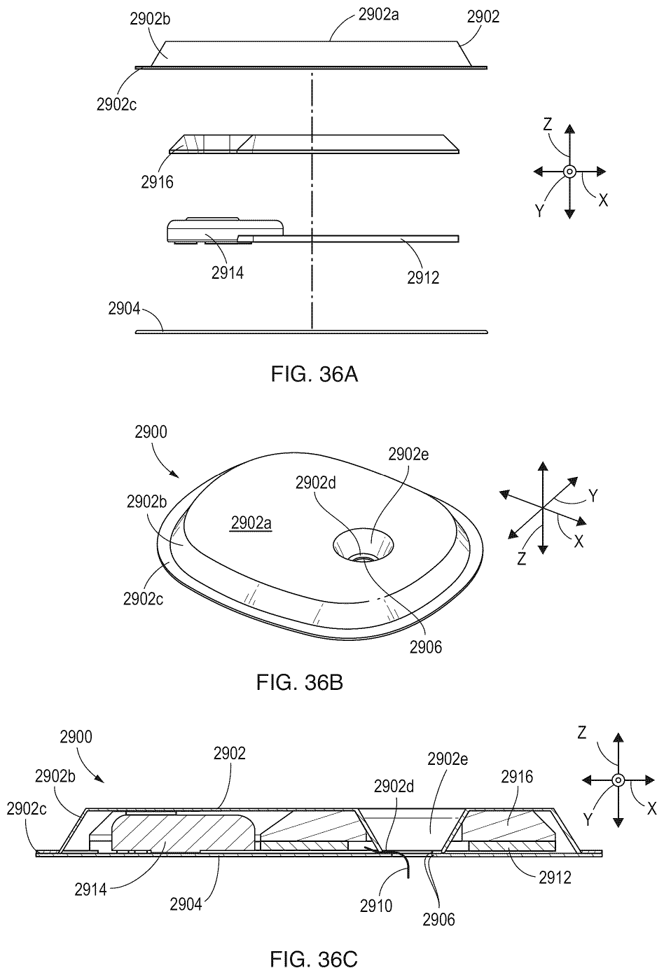

Referring to FIGS. 36A to 36C, in some embodiments, the continuous analyte monitoring system 104 includes a housing 2900 including an upper housing 2902 and a lower housing 2904, the lower housing 2904 interfacing with the skin of the user during use, for example through an adhesive layer or patch. The upper housing 2902 and lower housing 2904 may cooperate to define an opening 2906 for receiving a needle used to insert a portion or the sensor into the skin of the user. The upper housing 2902 may be made of a rigid material whereas the lower housing 2904 is made of a flexible material, such as a thin (e.g., less than 0.5 or 0.1 mm) membrane or a foam. For example, the lower housing 2904 may be made of a material having a hardness of less than or equal to 50 Shore A, less than or equal to 30 Shore A, or less than or equal to 20 Shore A. The upper housing 2902 may be made of a material having a hardness of greater than 30 Shore D, greater than 50 Shore D, or greater than 80 Shore D. The flexibility of the lower housing 2904 is particularly beneficial for reducing likelihood of compression events.

The lower housing 2904 may be implemented as a flexible membrane or layer that is bonded to the upper housing 2902. The upper housing 2902 may also be implemented as a flexible membrane including the same material and possibly the same thickness as the lower housing 2904. The upper housing 2902 may include a central portion 2902a that is substantially planar and a sidewall 2902b that extends around the central portion 2902a and extends downwardly from the central portion in the Z direction. A flange or rim 2902c may extend outwardly from the sidewall 2902b, such as parallel to the central portion 2902a. The lower housing 2904 may bond to the rim 2902c, such as by means of adhesive, plastic welding, or other fastening approach.

The opening 2906 in the upper housing 2902 may be formed in a bottom wall 2902d extending across an inner wall 2902e that extends downwardly from the central portion 2902a to the bottom wall 2902d. For example, the inner wall 2902e may have a cylindrical shape or the illustrated conical shape. The bottom wall 2902d may be bonded to the lower housing 2904 by means of adhesive, plastic welds, or other fastening approach.

In some embodiments, a sensor 2910 may pass from inside the housing, between the bottom wall 2902d and the lower housing 2904, and into the opening 2906 such that the sensor 2910 may be engaged by a needle during the insertion process.

As shown in FIG. 36C, the bonding between the upper housing 2902 and the lower housing 2904 provides a sealed chamber that protects components within the housing 2900 from moisture or contamination. The sealed chamber may contain a circuit board 2912 coupled to the sensor 2910 and battery 2914 coupled to the circuit board 2912.

In some embodiments, some or all of a remaining volume within the upper housing 2902 and the lower housing 2904 that is not occupied by other components, such as the circuit board 2912 and battery 2914 may be occupied by a filler 2916, such as a structural foam, a polymer, or an elastomer. For example, the filler 2916 may occupy between 70 and 100 percent of the remaining volume. The filler may have properties selected to (a) reduce the impact of compression events by facilitating flexibility of the combined housing 2900 and filler 2916 and (b) provide toughness to avoid damage to the circuit board 2912 and sensor 2910. The filler 2916 may be made of polyurethane, polystyrene, polyethylene, cross-linked polyethylene, or silicone. Where intended to be flexible, the filler 2916 may have a hardness of less than or equal to 50 Shore A, less than or equal to 30 Shore A, or less than or equal to 20 Shore A. Where intended to provide toughness, the filler 2916 may have a hardness of greater than 30 Shore D, greater than 50 Shore D, or greater than 80 Shore D.

The housing of FIG. 36C may be assembled by first assembling the circuit board 2912, securing the sensor 2910 and battery 2914 to the circuit board 2912, placing the board between the upper housing 2902 and the lower housing 2904, bonding the upper housing 2902 to the lower housing 2904, and sealing the sensor 2910 between the upper housing 2902 (e.g., using epoxy) and lower housing 2904.

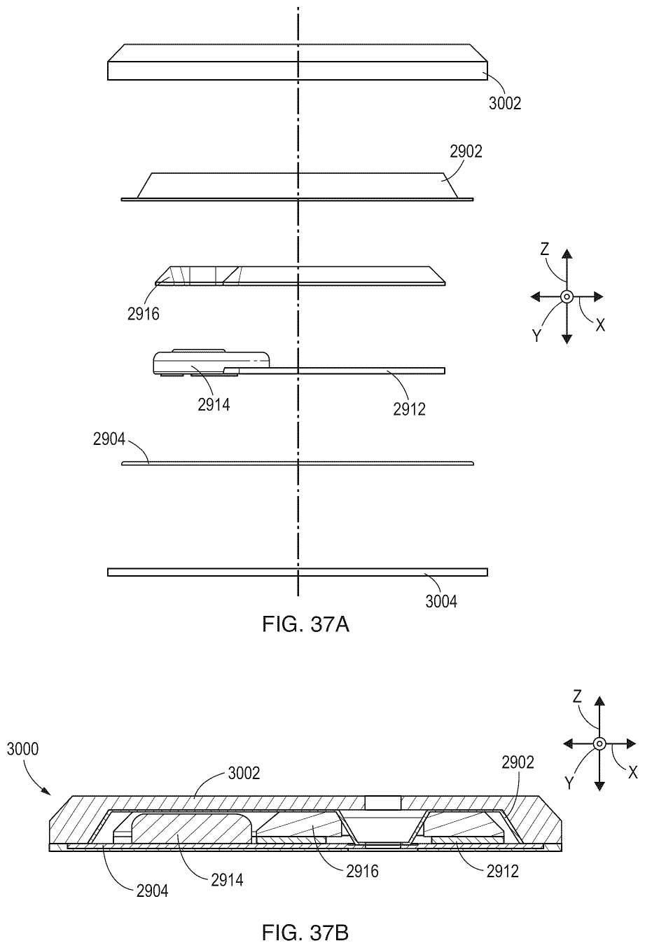

Referring to FIGS. 37A and 37B, in some embodiments, the housing 2900 may be contained within an outer housing 3000. The housing 2900 may provide containment and sealing whereas the outer housing provides mechanical support. The properties of the housing 2900 and the outer housing 3000 may therefore be decoupled, providing greater design freedom in selecting and achieving the mechanical properties of the outer housing. In the embodiment of FIGS. 37A and 37B, the upper housing 2902 may be implemented as thin shell or membrane, such as less than 0.5 mm or less than 0.1 mm thick. For example, the upper housing 2902 may be made of the same material and have the same thickness of the lower housing 2904.

The outer housing 3000 may include an upper outer housing 3002 extending over the central portion 2902a and sidewall 2902b of the upper housing 2902. A lower outer housing 3004 may extend over the lower housing 2904. The upper outer housing 3002 may be bonded to the one or both of the upper housing 2902, which may include the rim 2902c, and the lower outer housing 3004. Likewise, the lower outer housing 3004 may be bonded to one or both of the lower housing 2904 and the upper outer housing 3002.

In some embodiments, the upper outer housing 3002 and lower outer housing 3004 are made of a flexible and/or compressible material, such as a structural foam, polymer, or elastomer. The density, modulus of elasticity, and thickness of the flexible and/or compressible material constituting the upper outer housing 3002 and lower outer housing 3004 may be selected to reduce the transmission of compressive loads to the tissue around the insertion site. The upper housing can comprise the same material, or a different material or combination of materials, as the lower housing.

For example, the upper and/or lower outer housing 3004 may be made of structural foam as used in any of the embodiments described hereinabove. The structural foam may be an open or closed cell foam. The structural foam of the lower outer housing 3004 may conduct moisture and function to conduct moisture away from the lower housing 2904. The upper outer housing 3002 may have a greater thickness in the Z direction than the lower outer housing 3004, such as at least 2, 4, or 8 times. The upper outer housing 3002 may have a thickness of between 0.5 and 2 mm.

In some embodiments, the upper outer housing 3002 is made of flexible or rigid material having very low friction. For example, the upper outer housing 3002 may be made of a plastic having a coefficient of friction of less than 0.4, 0.3, 0.2, or 0.15. For example, the upper outer housing 3002 may be made of high-density polyethylene (HDPE), polypropylene (PP), nylon, or other polymer. In contrast, the lower outer housing 3004 may be made of a flexible material selected to provide good adhesion with an adhesive patch used to adhere the lower outer housing 3004 to the skin of the user. Providing flexibility in the lower outer housing 3004 can reduce stress at the interface between the lower outer housing 3004 and the adhesive patch secured to the skin of the user. For example, the lower outer housing 3004 may be made of a material having a hardness of less than or equal to 50 Shore A, less than or equal to 30 Shore A, or less than or equal to 20 Shore A. The upper outer housing 3002 may be made of a material having a hardness of greater than 30 Shore D, greater than 50 Shore D, or greater than 80 Shore D.

The methods disclosed herein include one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

The term “continuous,” as used herein, is a broad term, and is used in its ordinary sense, and can mean continuous, semi-continuous, continual, periodic, intermittent, regular, etc.

The terms “continuous analyte sensor,” “continuous multi-analyte sensor,” “continuous glucose sensor,” and “continuous lactate sensor,” as used herein, are broad terms, and are used in their ordinary sense, and refer without limitation to a device that continuously measures a concentration of an analyte or calibrates the device (e.g., by continuously adjusting or determining the sensor's sensitivity and background), for example, at time intervals ranging from fractions of a second up to, e.g., 1, 2, or 5 minutes, or longer.

The terms “sensitivity” or “sensor sensitivity,” as used herein, are broad terms, and are used in their ordinary sense, and refer without limitation to an amount of signal produced by a certain concentration of a measured analyte, or a measured species (e.g., H₂O₂) associated with a measured analyte (e.g., glucose or lactate). For example, a sensor may have a sensitivity of from about 1 to about 300 pico Amperes (pA) of current for every 1 mg/dL of glucose analyte.

The term “sensor data,” as used herein, is a broad term, and is used in its ordinary sense, and refers without limitation to any data associated with a sensor, such as a continuous analyte or continuous multi-analyte sensor. Sensor data includes a raw data stream, or simply data stream, of analog or digital signal directly related to a measured analyte from an analyte sensor (or other signal received from another sensor), as well as calibrated or filtered raw data. The terms “sensor data point” and “data point” refer generally to a digital representation of sensor data at a particular time. The terms broadly encompass a plurality of time spaced data points from a sensor, such as a continuous analyte sensor, which includes individual measurements taken at time intervals ranging from fractions of a second up to, e.g., 1, 2, or 5 minutes or longer. In another example, the sensor data includes an integrated digital value representative of one or more data points averaged over a time period. Sensor data may include calibrated data, smoothed data, filtered data, transformed data, or any other data associated with a sensor.

The term “sensor electronics,” as used herein, is a broad term, and is used in its ordinary sense, and refers without limitation to components, e.g., hardware or software, of a device configured to process sensor data.

Although certain embodiments herein are described with reference to management of diabetes, diabetes management is only an example of one application for which the present systems and methods may be utilized. The systems and methods described herein can also be used for managing one or more other diseases or conditions, which may or may not include diabetes. For example, the systems and methods described herein can be utilized for managing kidney disease, liver disease, and other types of diseases or conditions.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “including” as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. Δt the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. An on-skin wearable medical device comprising:
a housing configured to be worn over skin of a user;
one or more electronic components disposed within the housing;
one or more analyte sensors electrically connected to the one or more electronic components and configured to sense one or more analytes within tissue of a user; and
a force sensor operably coupled to the housing and electrically connected to the one or more electronic components.

2. The on-skin wearable medical device of claim 1, wherein the one or more electronic components comprise a controller configured to adjust one or more outputs of the one or more analyte sensors according to an output of the force sensor.

3. The on-skin wearable medical device of claim 1, wherein the force sensor is under a preload in the absence of an external load on the housing.

4. The on-skin wearable medical device of claim 1, wherein the force sensor comprises at least one of a force sensitive resistor (FSR), an inductive force sensor, a capacitive force sensor, and a strain gauge.

5. (canceled)

6. (canceled)

7. The on-skin wearable medical device of claim 1, wherein the force sensor comprises an FSR, and the FSR comprises first and second electrodes and a conductive member extending over the first and second electrodes, the conductive member having pressure-sensitive conductance.

8. The on-skin wearable medical device of claim 7, wherein the first and second electrodes each include a plurality of fingers in an interdigitated configuration.

9. The on-skin wearable medical device of claim 7, wherein the conductive member is compressible.

10. (canceled)

11. The on-skin wearable medical device of claim 7, wherein the conductive member comprises a curved lower surface.

12. (canceled)

13. The on-skin wearable medical device of claim 7, wherein the conductive member includes a first layer and a second layer coupled to the first layer, wherein one or more of the first layer and the second layer is compressible.

14. The on-skin wearable medical device of claim 13, wherein the second layer is bonded to the first layer.

15.-18. (canceled)

19. The on-skin wearable medical device of claim 7, wherein the FSR includes an adhesive tape coupled to an outer surface of the conductive member that is opposite the first and second electrodes, the adhesive tape coupling the conductive member to a substrate that the first and second electrodes are positioned on.

20.-22. (canceled)

23. The on-skin wearable medical device of claim 1, further comprising a concentrator configured to transfer at least a portion of a minimum external load from the housing to at least a portion of the force sensor.

24. The on-skin wearable medical device of claim 23, wherein the concentrator comprises a protrusion extending from an upper portion of the housing.

25. (canceled)

26. The on-skin wearable medical device of claim 23, wherein the concentrator has a cross-sectional dimension that is no larger than a dimension of an active area of the force sensor.

27. (canceled)

28. The on-skin wearable medical device of claim 1, wherein the one or more electronic components comprise a circuit board and the force sensor is disposed between the circuit board and an upper or lower portion of the housing.

29. (canceled)

30. The on-skin wearable medical device of claim 1, further comprising at least one support operably coupled to the housing, and wherein the at least one support is configured to support the force sensor.

31. The on-skin wearable medical device of claim 1, further comprising at least one support operably coupled to the housing, and wherein the one or more electronic components comprise a circuit board and wherein the at least one support is configured to support the circuit board.

32.-36. (canceled)

37. The on-skin wearable medical device of claim 1, further comprising a compressible member disposed within the housing, and wherein the compressible member is configured to transfer at least a portion of a minimum external load from the housing to at least a portion of the force sensor.

38.-47. (canceled)

48. A method of detecting compressive forces on an on-skin wearable medical device, the method comprising:
providing a housing configured to be worn over skin of a user, the housing comprising a controller coupled to one or more analyte sensors configured to sense one or more analytes within tissue of the user;
providing a force sensor operably coupled to the housing and to the controller, the force sensor configured to sense loading of the housing;
receiving, by the controller, one or more outputs of the one or more analyte sensors;
receiving, by the controller, an output of the force sensor; and
adjusting, by the controller, the one or more outputs of the one or more analyte sensors according to the output of the force sensor.

49. (canceled)

50. The method of claim 48, wherein the force sensor comprises at least one of a force sensitive resistor (FSR), an inductive force sensor, a capacitive force sensor, and a strain gauge.

51.-150. (canceled)
